(19) United States
(12) Patent Application Publication
(10) Pub. No.: US 2022/0401564 A1
(43) Pub. Date: Dec. 22, 2022

(54) SELECTIVE HISTONE DEACETYLASE (HDAC) DEGRADERS AND METHODS OF USE THEREOF

(71) Applicant: DANA-FARBER CANCER INSTITUTE, INC., Boston, MA (US)

(72) Inventors: Eric S. Fischer, Chestnut Hill, MA (US); Yuan Xiong, Waltham, MA (US); Katherine Donovan, Brookline, MA (US); Nicholas Eleuteri, Braintree, MA (US)

(73) Assignee: DANA-FARBER CANCER INSTITUTE, INC., Boston, MA (US)

(21) Appl. No.: 17/774,002

(22) PCT Filed: Nov. 5, 2020
(86) PCT No.: PCT/US20/59111

§ 371 Date: May 3, 2022

Related U.S. Application Data
(60) Provisional application No. 62/931,533, filed on Nov. 6, 2019.

(51) Int. Cl. A61K 47/55; A61K 47/54

(52) U.S. Cl. CPC A61K 47/55; A61K 47/545

(57) ABSTRACT

The present invention relates to bifunctional compounds, compositions, and methods for treating diseases or conditions mediated by aberrant histone deacetylase (HCADC) (e.g., HDAC3) activity.

### RELATED APPLICATIONS

This application claims the benefit of priority under 35 U.S.C. § 119(e) to U.S. Provisional Application No. 62/931,533, filed on Nov. 6, 2019, which is incorporated herein by reference in its entirety.

### BACKGROUND OF THE INVENTION

The modification of histones by acetylation/deacetylation plays a key role in the regulation of gene expression by changing the structure of chromatin and by modulating the accessibility of transcription factors to their target DNA sequences (Eckschlager et al., Int. J. Mol. Sci. 18:1414 (2017)). The acetylation state of histones and other proteins are maintained by histone acetyltransferases (HAT) and histone deacetylases (HDAC). HATs add acetyl groups to lysine residues, while HDACs remove the acetyl groups. Generally, the acetylation of histone promotes a more relaxed chromatin structure which allows for transcriptional activation (Xu et al., Oncogene 26:5541-5552 (2007)). In addition to regulating histone modification, HDACs also regulate the post-translational acetylation of many non-histone proteins, including transcription factors, chaperones, and signaling molecules, resulting in changes in protein stability, protein-protein interactions, and protein-DNA interactions (Glozak et al., Gene 363:15-23 (2005)). The balance between histone acetylation and deacetylation is usually well regulated, but the balance is often upset in diseases such as cancer and neurodegenerative diseases.

HDACs are composed of 18 members (isoforms) that are divided into four classes based on their homology. There are 11 conventional HDACs that require Zn²⁺ as a cofactor for their deacetylase activity; they fall within classes I, II, and IV. Class I HDACs, which includes HDACs 1, 2, 3, and 8, are located only within the nucleus and are related to yeast RPD3 gene. Class II includes HDACs 4, 5, 6, 7, 9, and 10 which are located in both the nucleus and the cytoplasm, and are related to yeast Hda1 gene. Class IV includes HDAC 11 and has features of both Class I and Class II HDACs. Unlike conventional HDACs, Class III HDACs are composed of seven mammalian sirtuins (SIRT1-7), which include nicotinamide adenine dinucleotide (NAD⁺)-dependent protein deacetylases localized in the nucleus (SIRT1, SIRT6, and SIRT7), mitochondria (SIRT3, SIRT4, and SIRT5), and cytoplasm (SIRT2) (Kim et al., Am. J. Transl. Res. 3:166-179 (2011)).

In view of the many HDAC isoforms, HDAC inhibition has a narrow therapeutic window and an accompanying risk of causing several adverse side effects. Accordingly, there is a need for compounds that inhibit specific HDAC isoforms (e.g., HDAC3) while minimizing off-target toxicity caused by binding to other unintended HDAC isoforms, for use in treating diseases such as cancer and neurodegenerative diseases.

### SUMMARY OF THE INVENTION

A first aspect of the present invention is directed to a bifunctional compound, comprising an (E)-3-(4-(((2-(1H-indol-3-yl)ethyl)amino)methyl)phenyl)acrylamidyl moiety (also referred to herein as a “targeting ligand”) and a degron, covalently attached to each other by a linker, wherein the compound has a structure represented by formula (I) or (II):

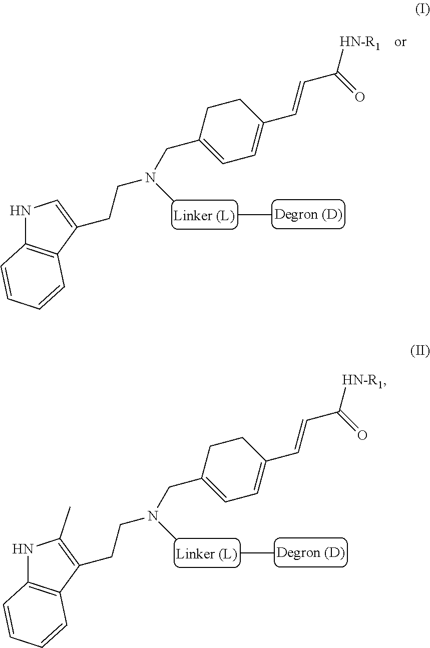

wherein R₁ is OH or

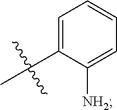 [SMILES: *c1ccccc1N]

and
the degron represents a ligand that binds von Hippel Landau tumor suppressor (VHL), or a pharmaceutically acceptable salt or stereoisomer thereof. In some embodiments, R₁ is OH.

Another aspect of the present invention is directed to a pharmaceutical composition containing a therapeutically effective amount of a bifunctional compound of formula (I) or (II) or a pharmaceutically acceptable salt or stereoisomer thereof, and a pharmaceutically acceptable carrier.

In another aspect of the present invention, methods of making the bifunctional compounds are provided.

A further aspect of the present invention is directed to a method of treating a disease or disorder characterized or mediated by aberrant HDAC3 activity, that includes administering a therapeutically effective amount of a bifunctional compound of formula (I) or (II) or a pharmaceutically acceptable salt or stereoisomer thereof, to a subject in need thereof.

As shown in working examples herein, bifunctional compounds embraced by formula (I) or (II) (also referred to herein as degraders) promote the degradation of HDAC isoforms in that unlike known inhibitors they do not target all HDAC isoforms. For example, bifunctional compounds 1-6, 8, and 10-12 are shown to promote degradation of HDAC3 while substantially sparing other HDAC isoforms. Bifunctional compound 7 is shown to promote degradation of HDAC isoforms 3, 6 and 8 (3/6/8) while substantially sparing other HDAC isoforms. By conjugating low nanomole potency of HDAC targeting ligands with an E3 ligase binder, the inventive bifunctional compounds (e.g., compounds 1-6, 8, and 10-12) are able to quickly recruit E3 ligase, and therefore promote the degradation of HDAC3.

Accordingly, the bifunctional compounds of the present invention may serve as a set of new chemical tools for HDAC knockdown, particularly HDAC3, exemplify a broadly applicable approach to arrive at degraders that are selective over non-selective binding ligands, and may provide effective treatments for HDAC-mediated diseases and disorders such as cancer, neurodegenerative diseases, and autoimmune diseases.

### BRIEF DESCRIPTION OF THE DRAWINGS

FIG. 1A-FIG. 1B are scatter plots that show the relative change in relative protein abundance with treatment of Kelly cells with inventive bifunctional compounds 1 (FIG. 1A) and 2 (FIG. 1B) compared to dimethyl sulfoxide (DMSO) control.

FIG. 2 is a heat map that shows the change in relative protein abundance of histone deacetylases (HDACs) identified in the experiment with treatment of Kelly cells with inventive bifunctional compounds 1 and 2 (1 μM, 5 hours), compared to DMSO control.

FIG. 3A-FIG. 3G are scatter plots that show the relative change in relative protein abundance with treatment of Kelly cells with inventive bifunctional compounds 7 (FIG. 3A), 8 (FIG. 3B), 3 (FIG. 3C), 5 (FIG. 3D), 10 (FIG. 3E), 11 (FIG. 3F), and 12 (FIG. 3G) compared to dimethyl sulfoxide (DMSO) control.

FIG. 4A-FIG. 4B are graphs of cellular VHL engagement assays with inventive bifunctional compounds 1, 2, 3, 4, 5, 9, and 10 (FIG. 4A), and bifunctional compounds 7 and 8, and control (FIG. 4B).

### DETAILED DESCRIPTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “alkoxylene” refers to a saturated monovalent aliphatic radicals of the general formula (—O—CₙH₂ₙ—) where n represents an integer (e.g., 1, 2, 3, 4, 5, 6, or 7) and is inclusive of both straight-chain and branched-chain radicals. The alkoxylene chain may be attached to the rest of the molecule through a single bond and to the radical group through a single bond. In some embodiments, the alkoxylene group contains one to 3 carbon atoms (—O—C₁-C₃ alkoxylene). In other embodiments, an alkoxylene group contains one to 5 carbon atoms (—O—C₁-C₅ alkoxylene).

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}— heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen that is independently optionally substituted. In another embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

The term heteroaryl also embraces N-heteroaryl groups which as used herein refers to a heteroaryl group, as defined above, and which contains at least one nitrogen atom and where the point of attachment of the N-heteroaryl group to the rest of the molecule is through a nitrogen atom in the heteroaryl group. The term heteroaryl further embraces C-heteroaryl groups which as used herein refer to a heteroaryl group as defined above and where the point of attachment of the heteroaryl group to the rest of the molecule is through a carbon atom in the heteroaryl group. The term heteroaryl further embraces heteroarylalkyl groups which as disclosed above refer to a group of the formula —R^{c}-heteroaryl, wherein R^{c} is an alkylene chain as defined above. The term heteroaryl further embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The term “binding” as it relates to interaction between the targeting ligand and the targeted protein, which in this invention may be histone deacetylase 3 (HDAC3), typically refers to an inter-molecular interaction that may be selective (also referred to herein as “preferential”) in that the bifunctional compounds discriminate among targets. The binding of the targeting ligand with other HDAC isoforms, is about no greater than or less than or substantially less than, and in some cases functionally insignificant, at least from the standpoint of degradation. Selective HDAC3 degraders (e.g., compounds 1-6, 8, and 10-12) described herein may have a DC₅₀ (half maximal degradation concentration) for HDAC3 activity that is at least about 1, 2, 3, 4, 5, 6, 7, 8, 9, or 10-fold lower than the DC₅₀ for one or more of HDAC1, HDAC2, HDAC4, HDAC5, HDAC7, HDAC9, and/or HDAC10.

The term “binding” as it relates to interaction between the degron and the E3 ubiquitin ligase, typically refers to an inter-molecular interaction that may or may not exhibit an affinity level that equals or exceeds that affinity between the targeting ligand and the target protein, but nonetheless wherein the affinity is sufficient to achieve recruitment of the ligase to the targeted degradation and the selective degradation of the targeted protein.

Broadly, the bifunctional compounds comprise an (E)-3-(4-(((2-(1H-indol-3-yl)ethyl)amino)methyl)phenyl)acrylamidyl moiety and a degron covalently attached to each other by a linker, and have a structure represented by formula (I) or (II):

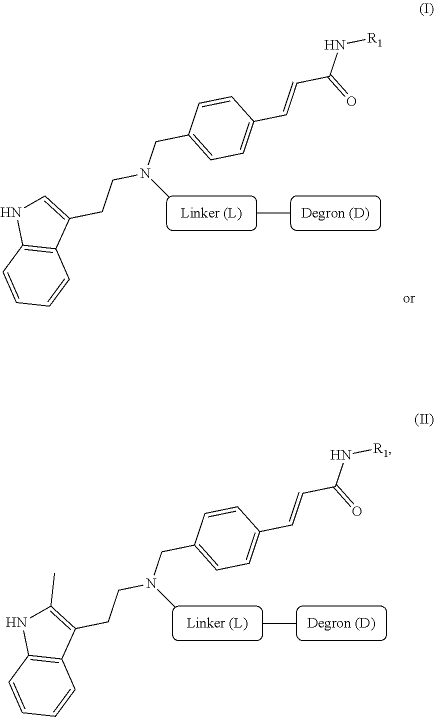

wherein R₁ is OH or

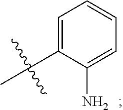 [SMILES: *c1ccccc1N]

and
the degron represents a ligand that binds von Hippel Landau tumor suppressor (VHL), or a pharmaceutically acceptable salt or stereoisomer thereof.

In some embodiments, R₁ is OH.

### Linkers

The linker (“L”) provides a covalent attachment between the targeting ligand and the degron. The structure of linker may not be critical, provided it does not substantially interfere with the activity of the targeting ligand or the degron. In some embodiments, the linker includes an alkylene chain (e.g., having 2-20 alkylene units).

In other embodiments, the linker may include an alkylene chain or a bivalent alkylene chain, either of which may be interrupted by, and/or terminate (at either or both termini) at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′) N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the linker may include a C₁-C₁₂ alkylene chain terminating in NH-group wherein the nitrogen is also bound to the degron.

In some embodiments, the linker includes an alkylene chain having 1-10 alkylene units that is interrupted by and/or terminating in

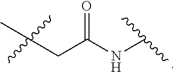 [SMILES: *CC(=O)N*]

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

Representative examples of alkylene linkers that may be suitable for use in the present invention include the following:

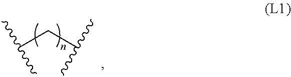

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

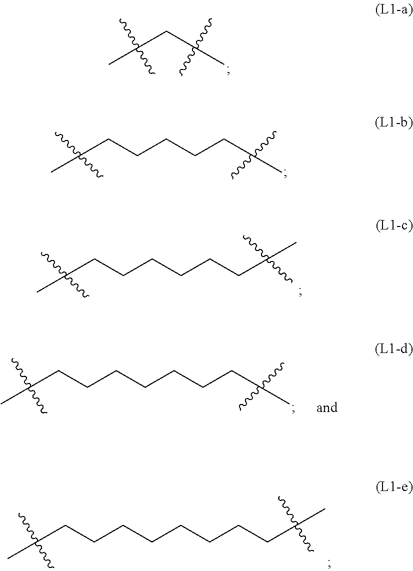

alkylene chains terminating in various functional groups (as described above), examples of which are as follows:

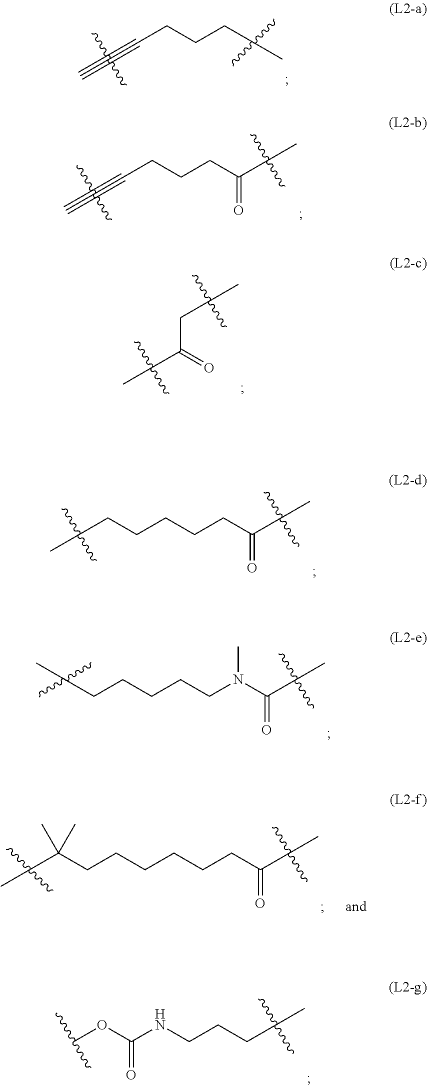

alkylene chains interrupted with various functional groups (as described above), examples of which are as follows:

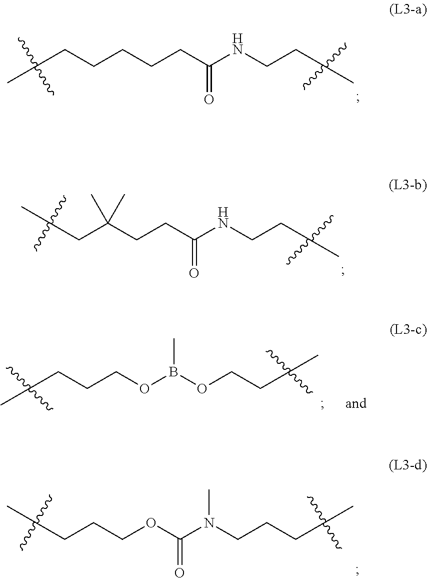 [SMILES: *CCCCCC(=O)NCC* ; *CCNC(=O)CCC(C)(C)C* ; *CCCOB(C)OCC* ; *CCCOC(=O)N(C)CCC*]

alkylene chains interrupted or terminating with heterocyclene groups, e.g.,

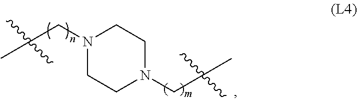

wherein m and n are independently integers of 0-10, examples of which include:

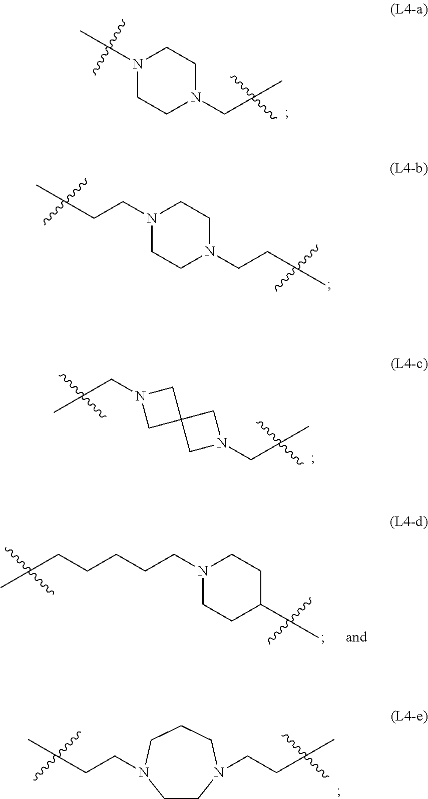 [SMILES: *CN1CCN(*)CC1 ; *CCN1CCN(CC*)CC1 ; *CN1CC2(C1)CN(C*)C2 ; *CCCCCN1CCC(*)CC1 ; *CCN1CCCN(CC*)CC1]

alkylene chains interrupted by amide, heterocyclene and/or aryl groups, examples of which include:

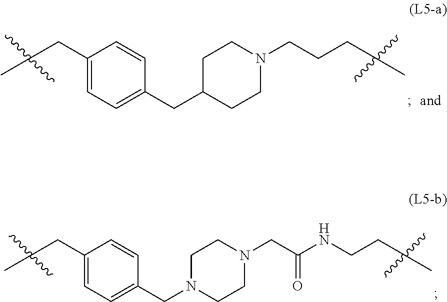

alkylene chains interrupted by heterocyclene and aryl groups, and a heteroatom, examples of which include:

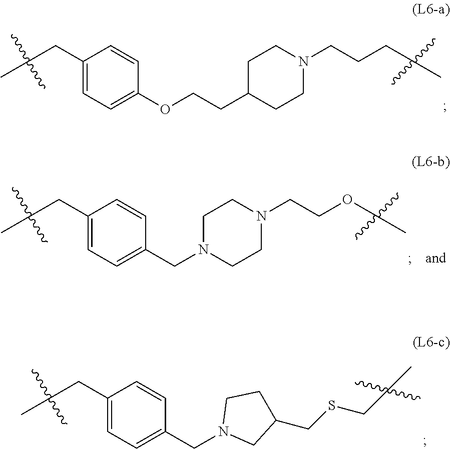 [SMILES: *CCCN1CCC(CCOc2ccc(C*)cc2)CC1 ; *Cc1ccc(CN2CCN(CCO*)CC2)cc1 ; *CSCC1CCN(Cc2ccc(C*)cc2)C1]

and
alkylene chains interrupted by a heteroatom such as N, O or B, e.g.,

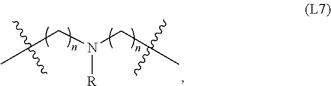

wherein each n is independently an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H or C₁ to C₄ alkyl, an example of which is

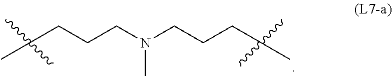 [SMILES: *CCCN(C)CCC*]

In some embodiments, the linker may include a polyethylene glycol chain that may terminate (at either or both termini) in at least one of —S—, —N(R′)—, —C≡C—, —C(O)—, —C(O)O—OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

In some embodiments, the linker includes a polyethylene glycol chain having 2-8 PEG units and terminating in

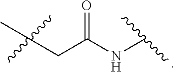 [SMILES: *CC(=O)N*]

Examples of linkers that include a polyethylene glycol chain include:

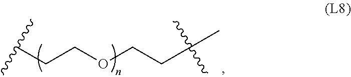

wherein n is an integer of 2-10, examples of which include:

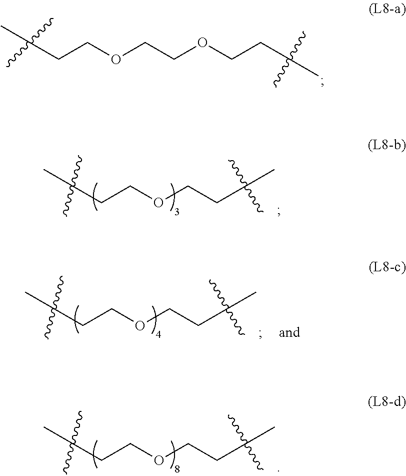

In some embodiments, the polyethylene glycol linker may terminate in a functional group, examples of which are as follows:

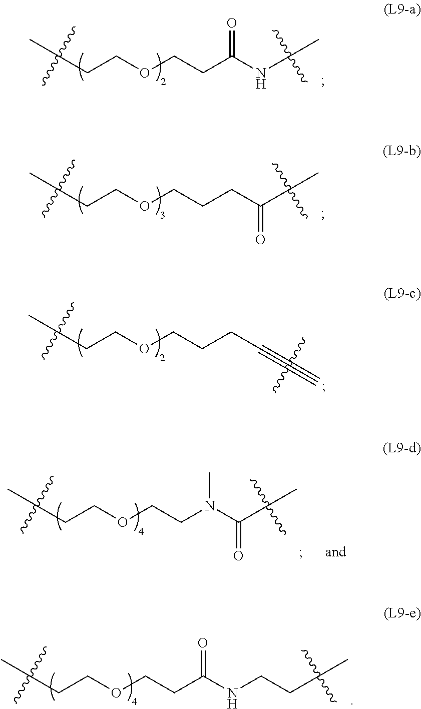

Thus, in some embodiments, bifunctional compounds of the present invention may be represented by any one of structures (I-1) to (I-7) and (II-1) to (II-7):

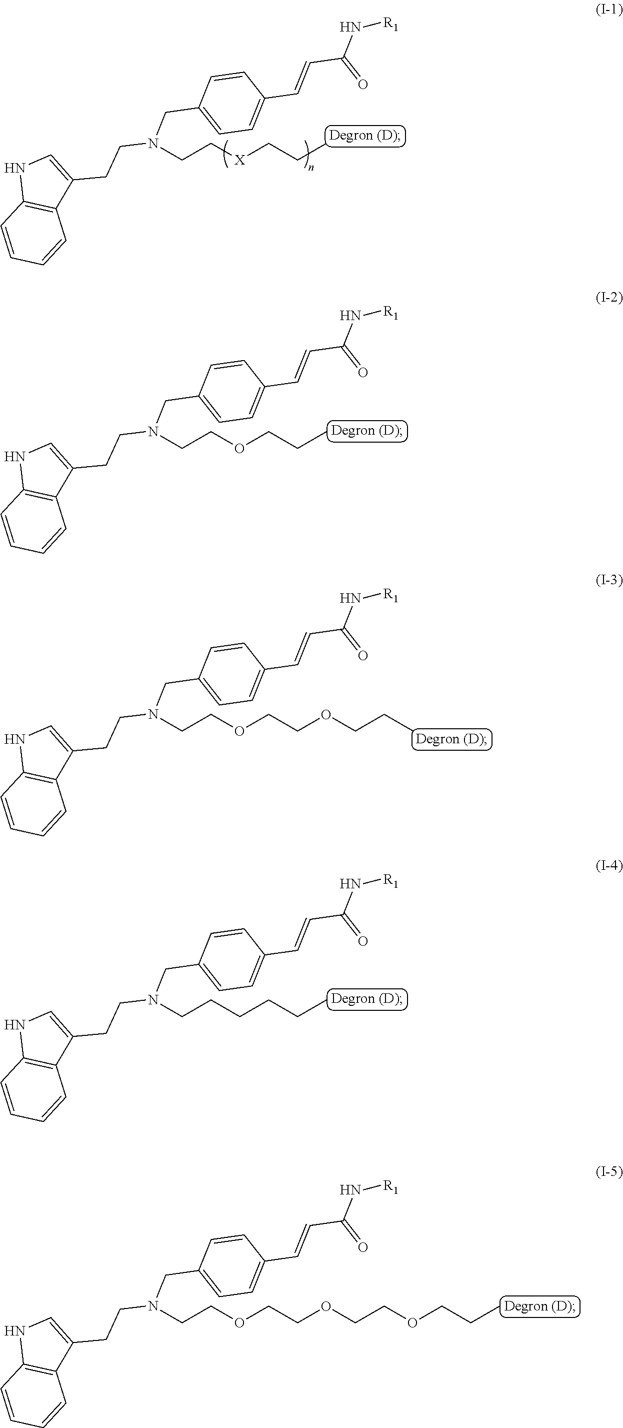

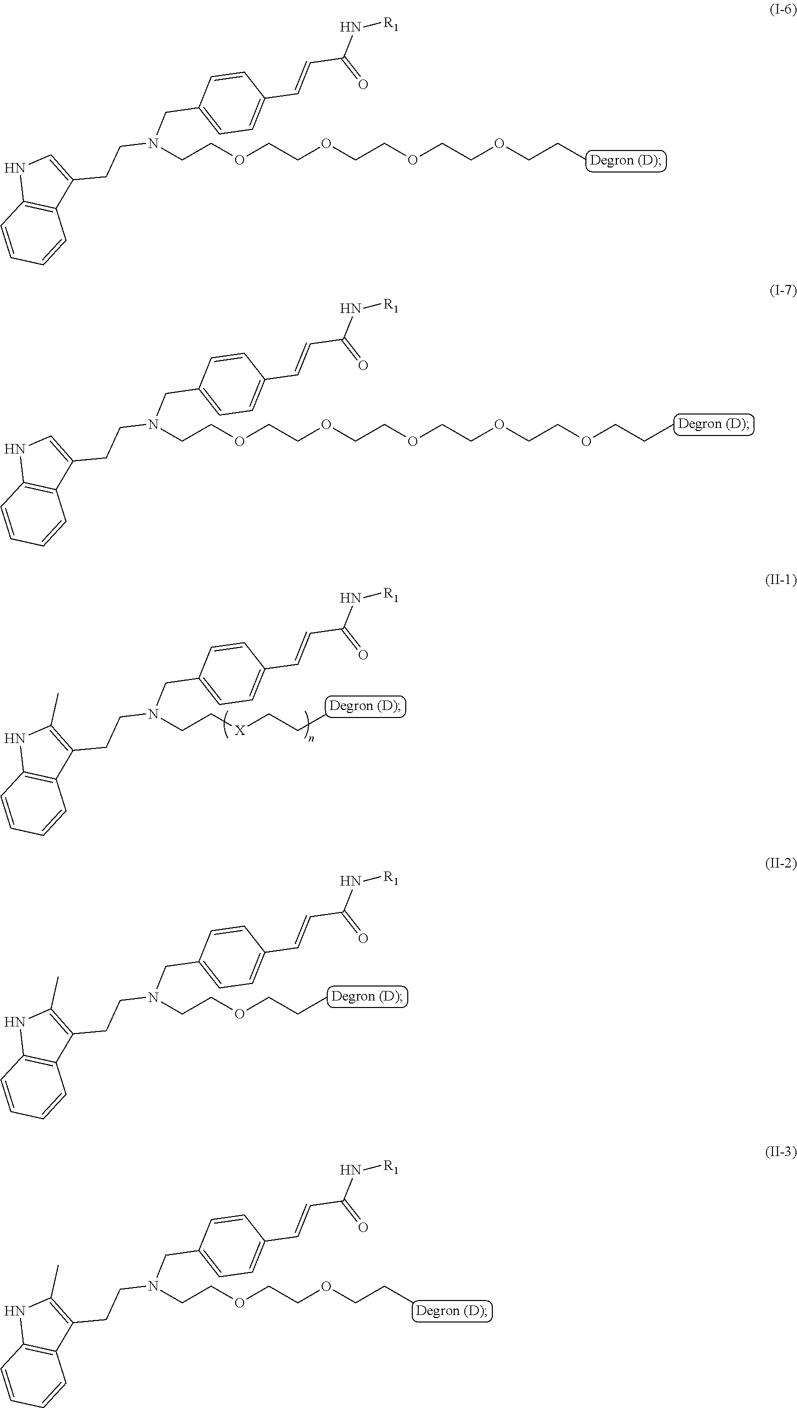

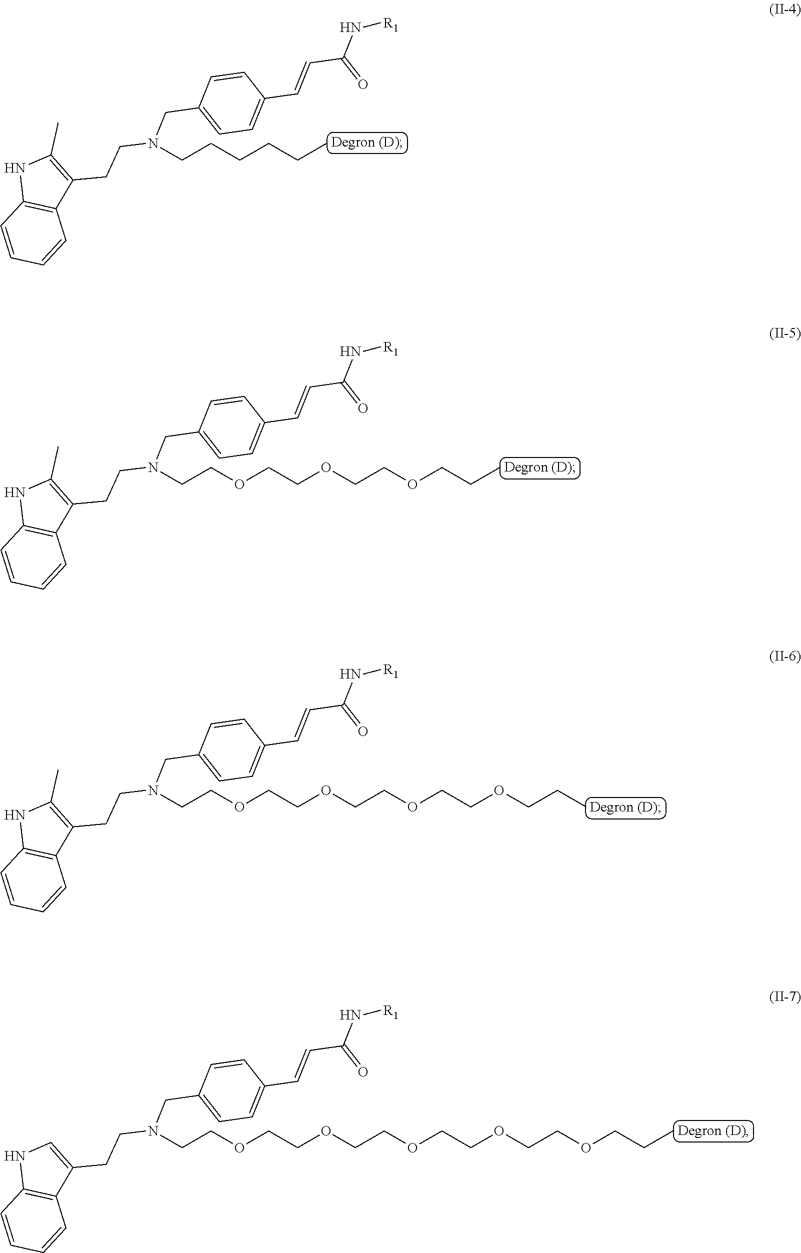

wherein n is an integer from 1-6; R₁ is OH or

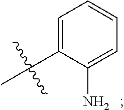 [SMILES: *c1ccccc1N]

and X is O or CH₂, or a pharmaceutically acceptable salt or stereoisomer thereof.

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

The degron binds the von Hippel-Lindau (VHL) E3 ubiquitin ligase.

Representative examples of such degrons may have structures represented by any one of structures (D1-a) to (D1-f):

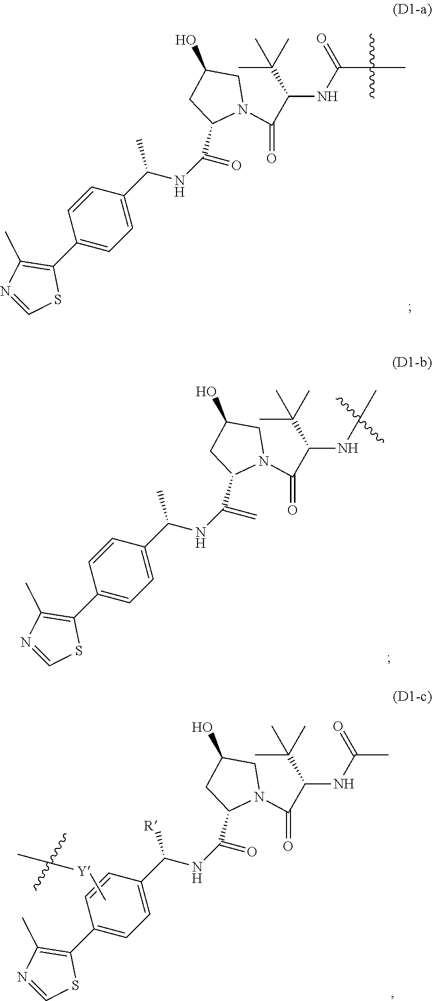

wherein Y′ is a bond, N, O or C and R′ is H or methyl;

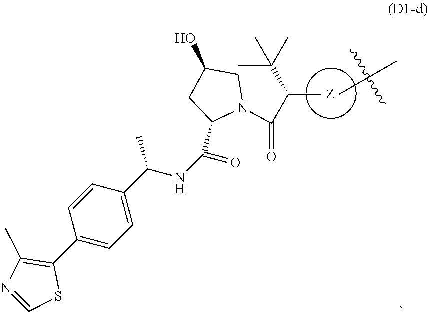

wherein Z is a cyclic group e.g., a C₅-C₆ carbocyclic or a C₅-C₆ heterocyclic group;

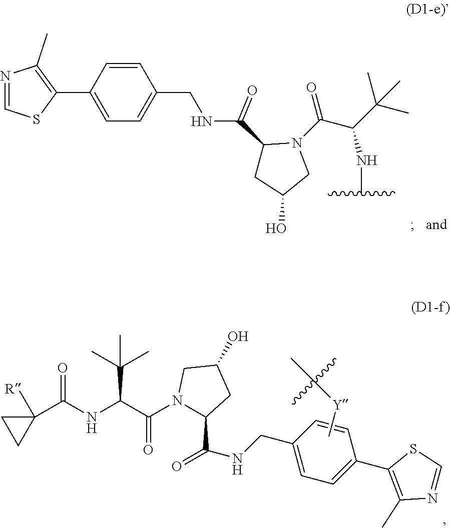

wherein Y″ is a bond, N, O or C and R″ is F or CN,
or stereoisomer thereof. In some embodiments, Z is

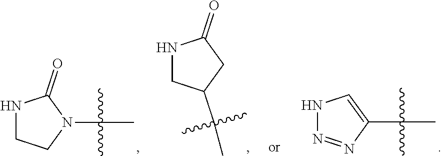 [SMILES: *N1CCNC1=O ; *C1CNC(=O)C1 ; *c1c[nH]nn1]

Yet other degrons that bind VHL and which may be suitable for use in the present invention are disclosed in U.S. Patent Application Publication 2017/0121321 A1.

Therefore, in some embodiments, the bifunctional compounds of the present invention may be represented by any one of structures (I-8) to (I-12) and (II-8) to (II-12):

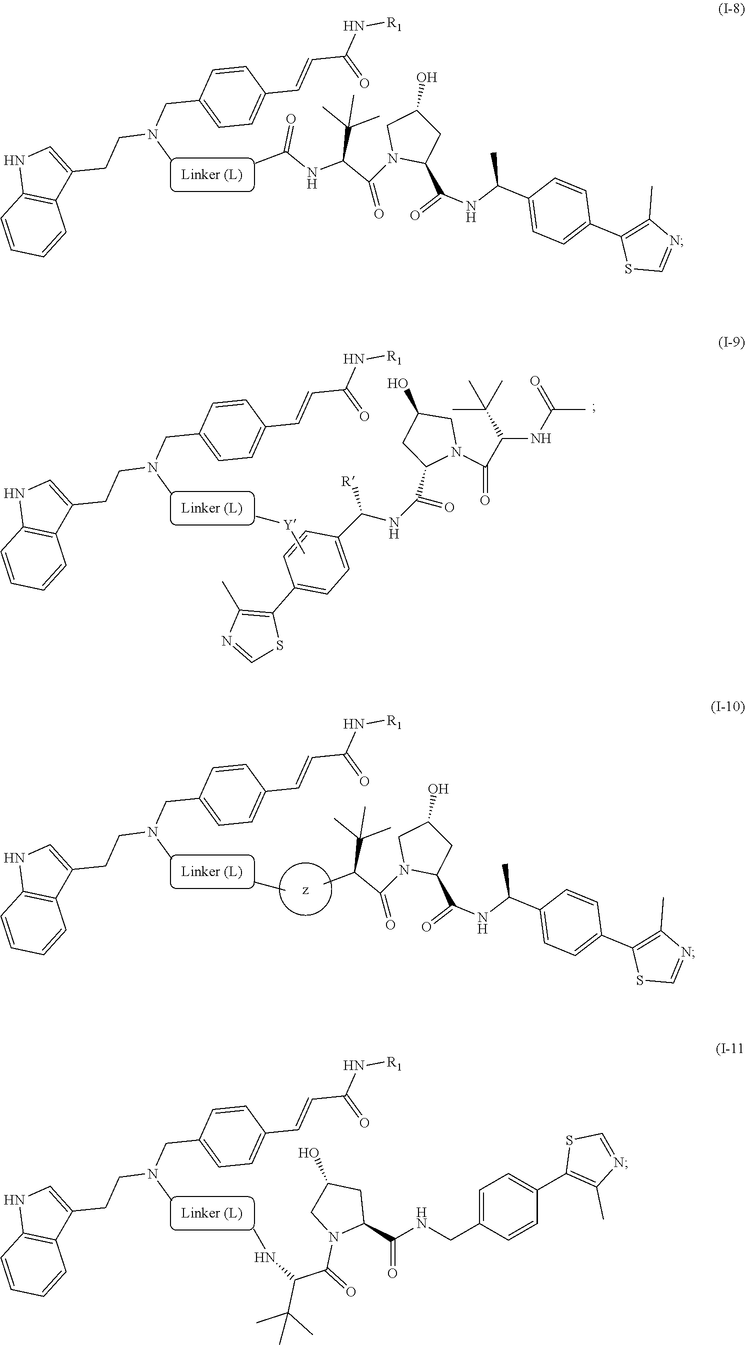

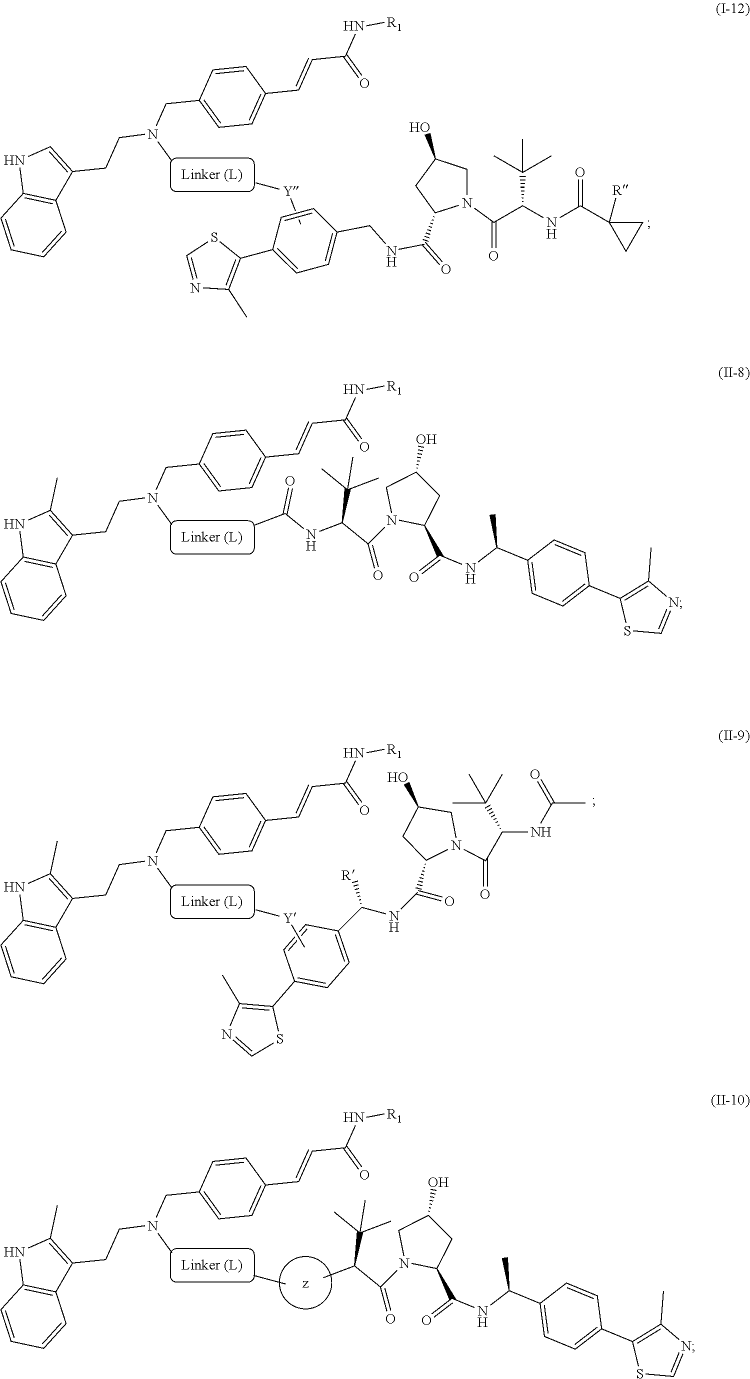

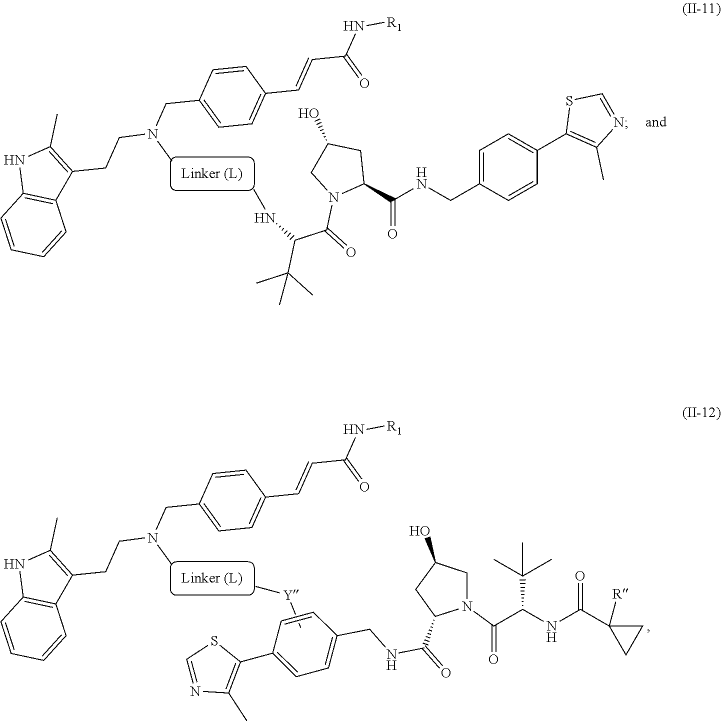

or a pharmaceutically acceptable salt or stereoisomer thereof.

In some embodiments, the bifunctional compounds of the present invention may be represented by any one of the structures (I-13) to (I-18) and (II-13) to (II-18):

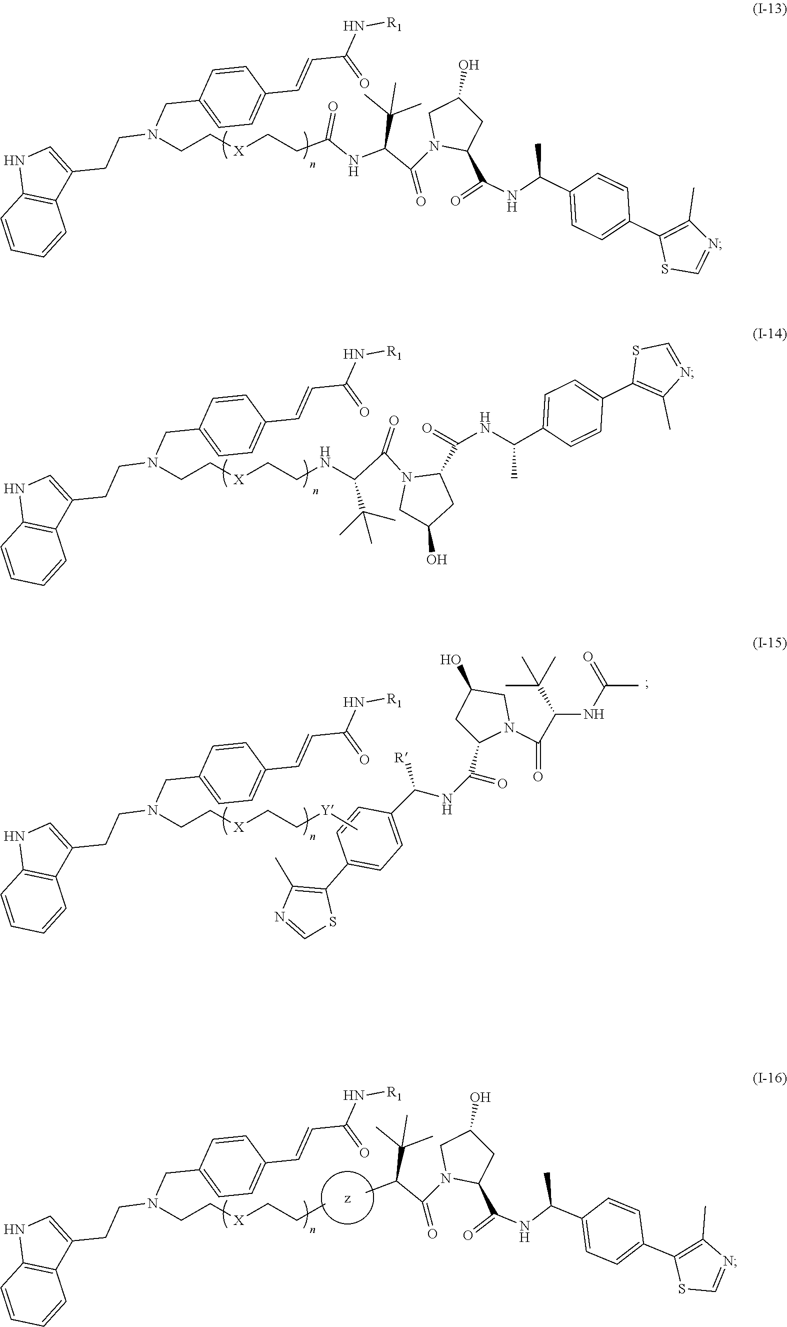

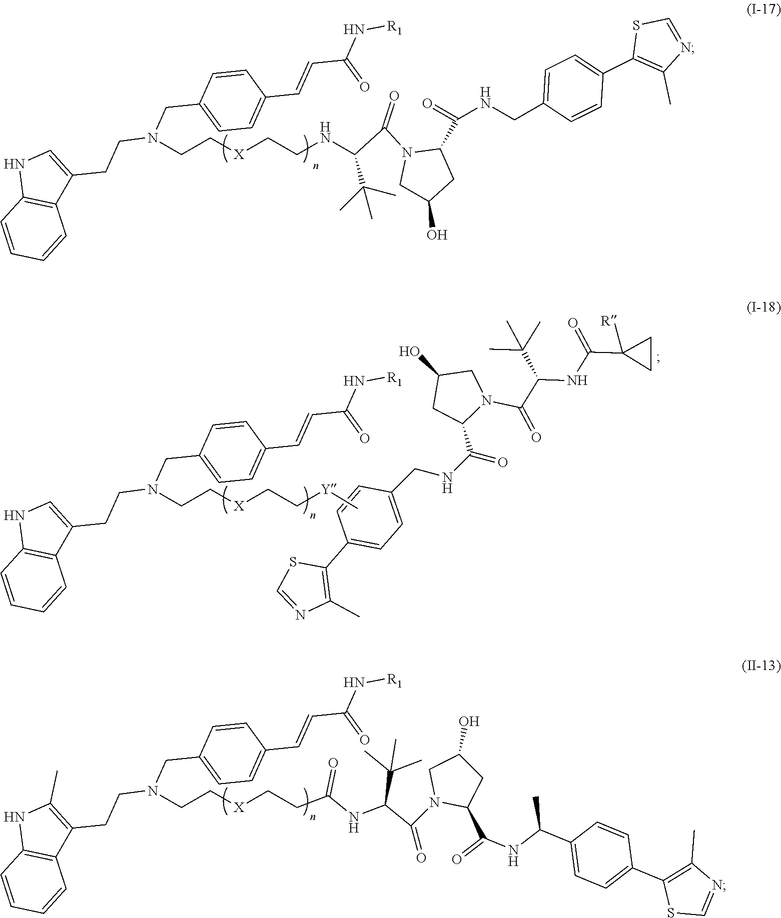

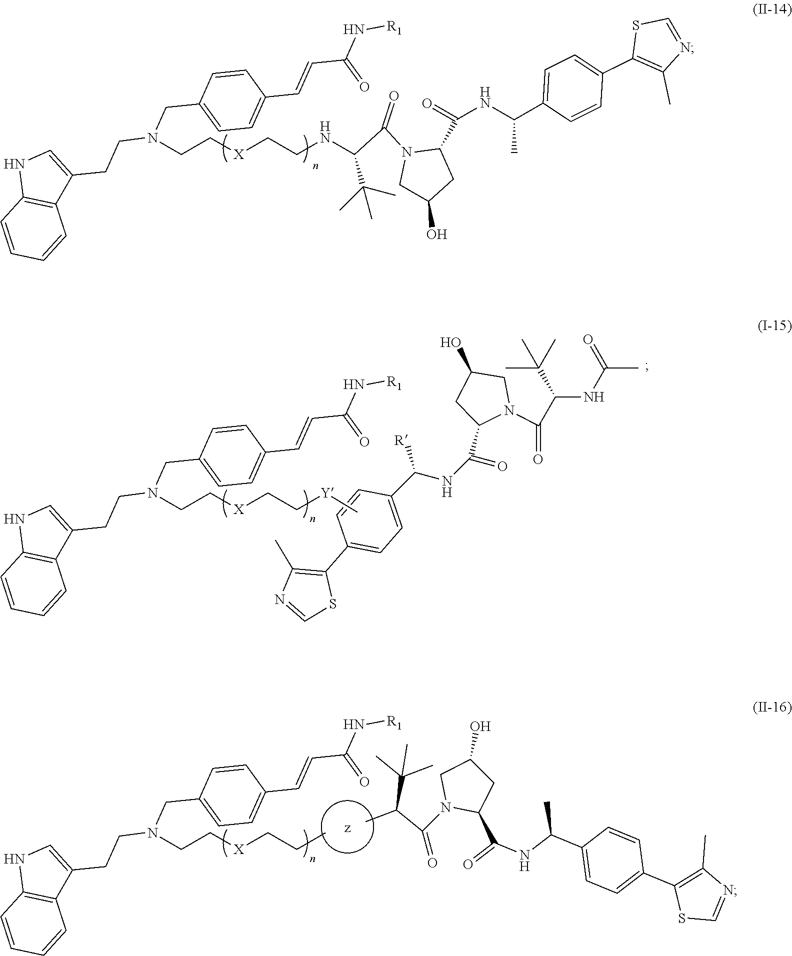

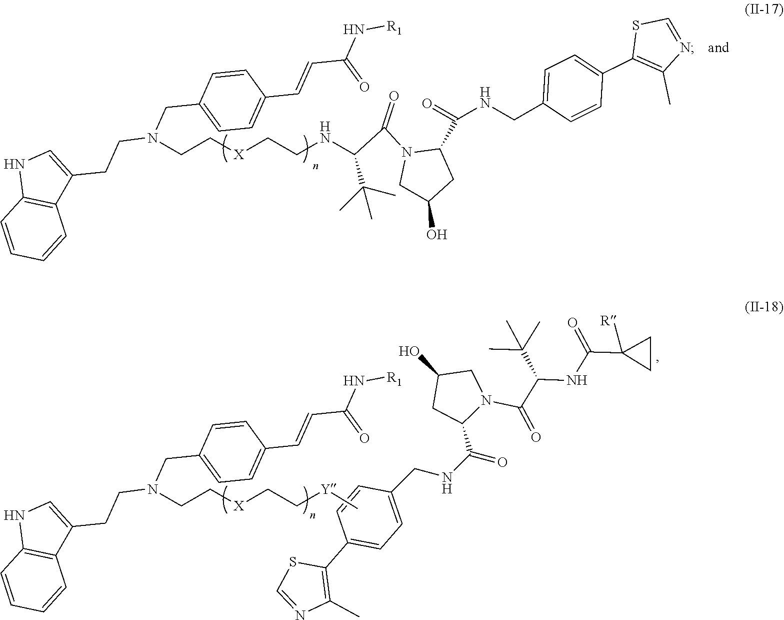

or a pharmaceutically acceptable salt or stereoisomer thereof.

In some embodiments, the bifunctional compounds of the present invention may have any one of the following structures:

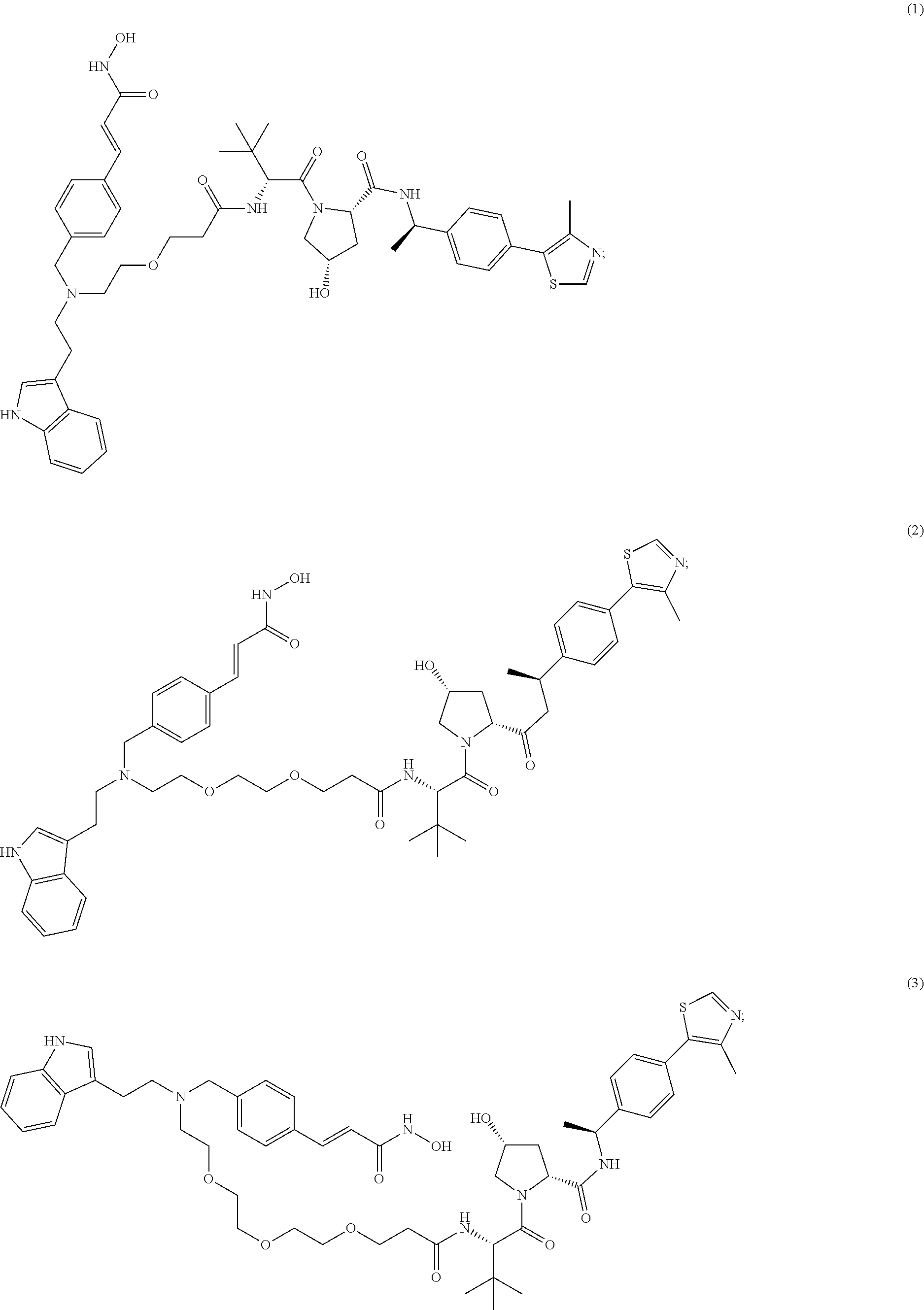 [SMILES: Cc1ncsc1-c1ccc([C@@H](C)NC(=O)[C@@H]2C[C@H](O)CN2C(=O)[C@H](NC(=O)CCOCCN(CCc2c[nH]c3ccccc23)Cc2ccc(/C=C/C(=O)NO)cc2)C(C)(C)C)cc1 ; Cc1ncsc1-c1ccc([C@H](C)CC(=O)[C@H]2C[C@@H](O)CN2C(=O)[C@@H](NC(=O)CCOCCOCCN(CCc2c[nH]c3ccccc23)Cc2ccc(/C=C/C(=O)NO)cc2)C(C)(C)C)cc1 ; Cc1ncsc1-c1ccc([C@H](C)NC(=O)[C@H]2C[C@@H](O)CN2C(=O)[C@@H](NC(=O)CCOCCOCCOCCN(CCc2c[nH]c3ccccc23)Cc2ccc(/C=C/C(=O)NO)cc2)C(C)(C)C)cc1]

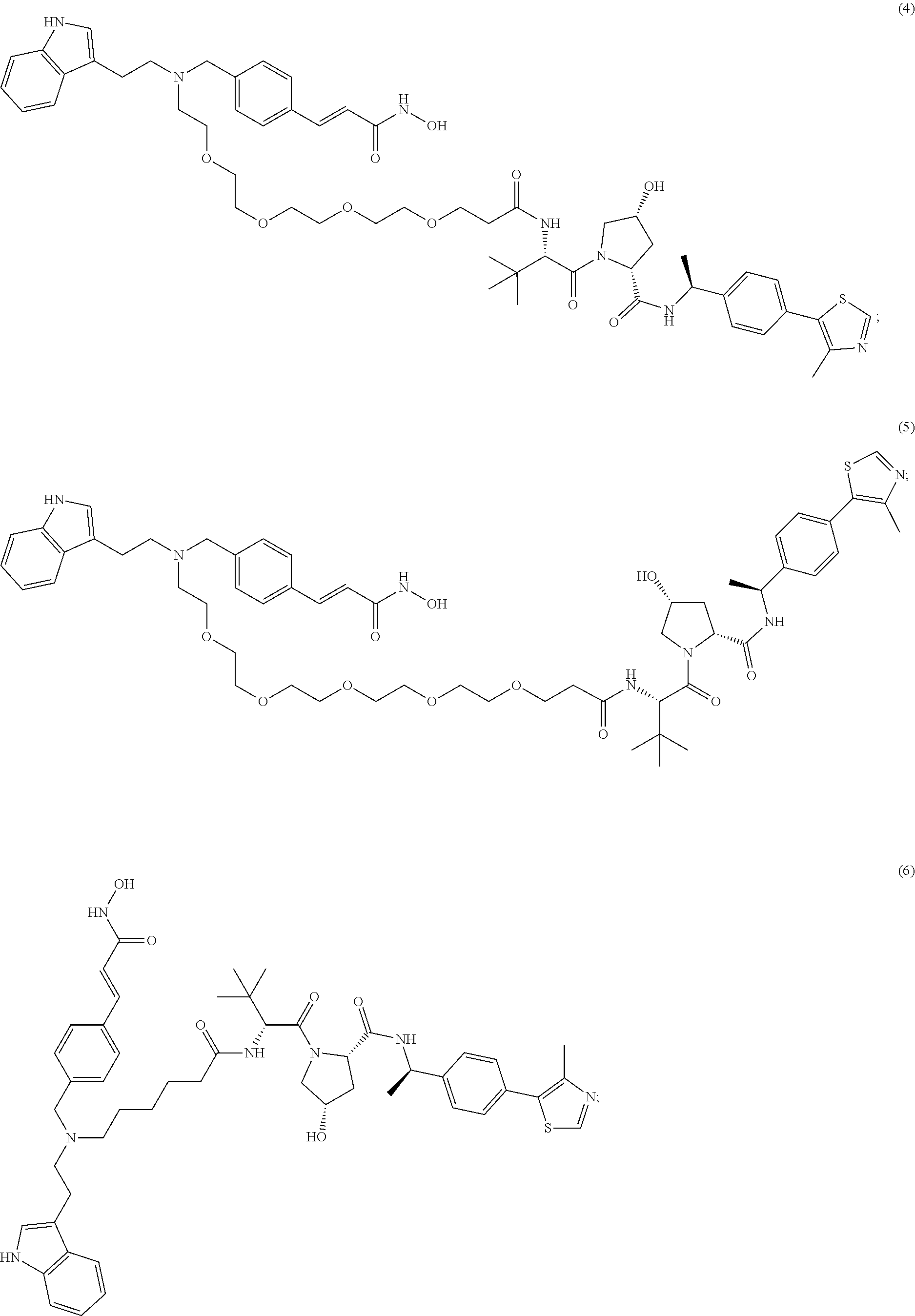 [SMILES: Cc1ncsc1-c1ccc([C@H](C)NC(=O)[C@H]2C[C@@H](O)CN2C(=O)[C@@H](NC(=O)CCOCCOCCOCCOCCN(CCc2c[nH]c3ccccc23)Cc2ccc(/C=C/C(=O)NO)cc2)C(C)(C)C)cc1 ; Cc1ncsc1-c1ccc([C@H](C)NC(=O)[C@H]2C[C@@H](O)CN2C(=O)[C@@H](NC(=O)CCOCCOCCOCCOCCOCCN(CCc2c[nH]c3ccccc23)Cc2ccc(/C=C/C(=O)NO)cc2)C(C)(C)C)cc1 ; Cc1ncsc1-c1ccc([C@@H](C)NC(=O)[C@@H]2C[C@H](O)CN2C(=O)[C@H](NC(=O)CCCCCN(CCc2c[nH]c3ccccc23)Cc2ccc(/C=C/C(=O)NO)cc2)C(C)(C)C)cc1]

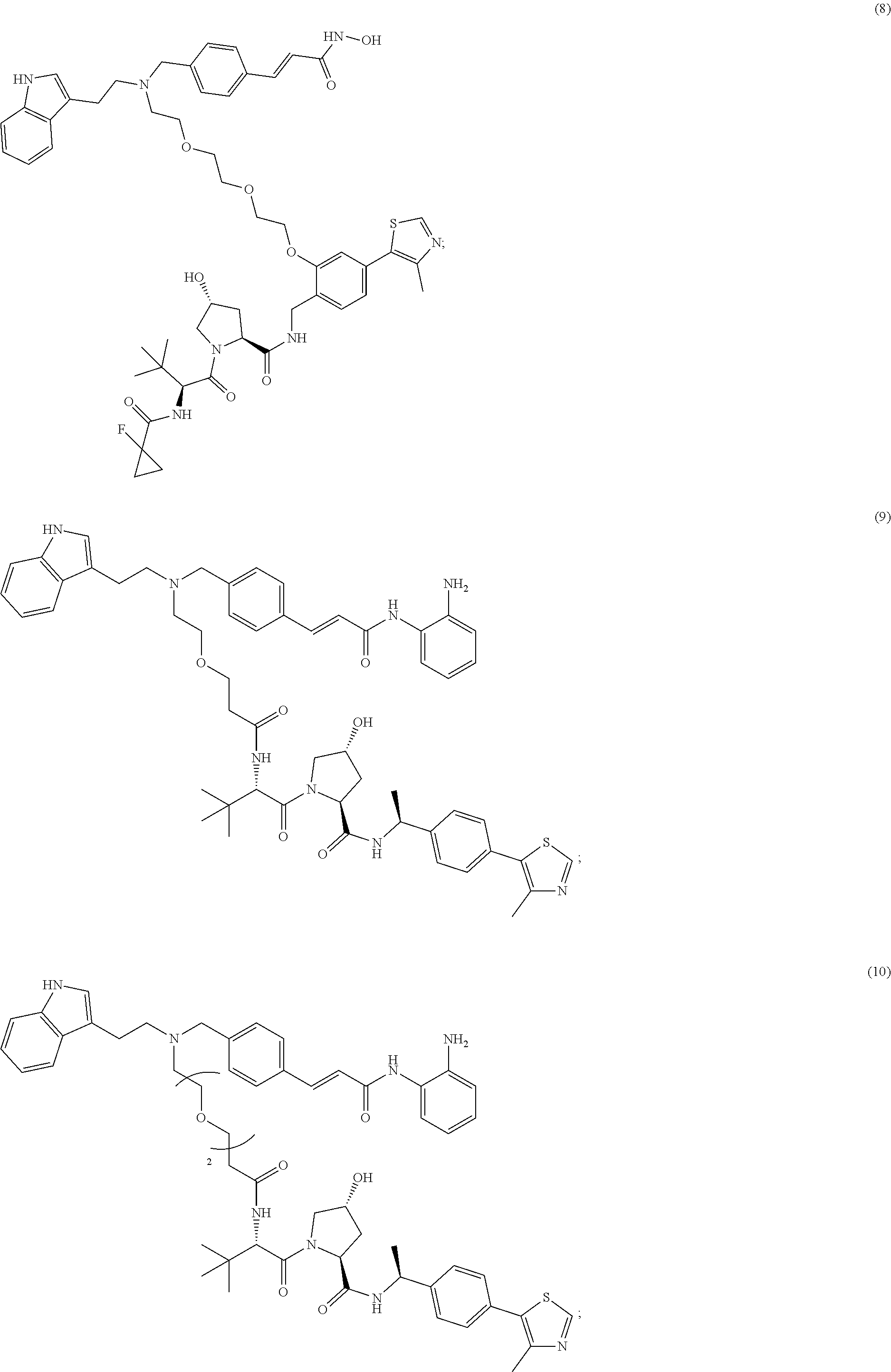

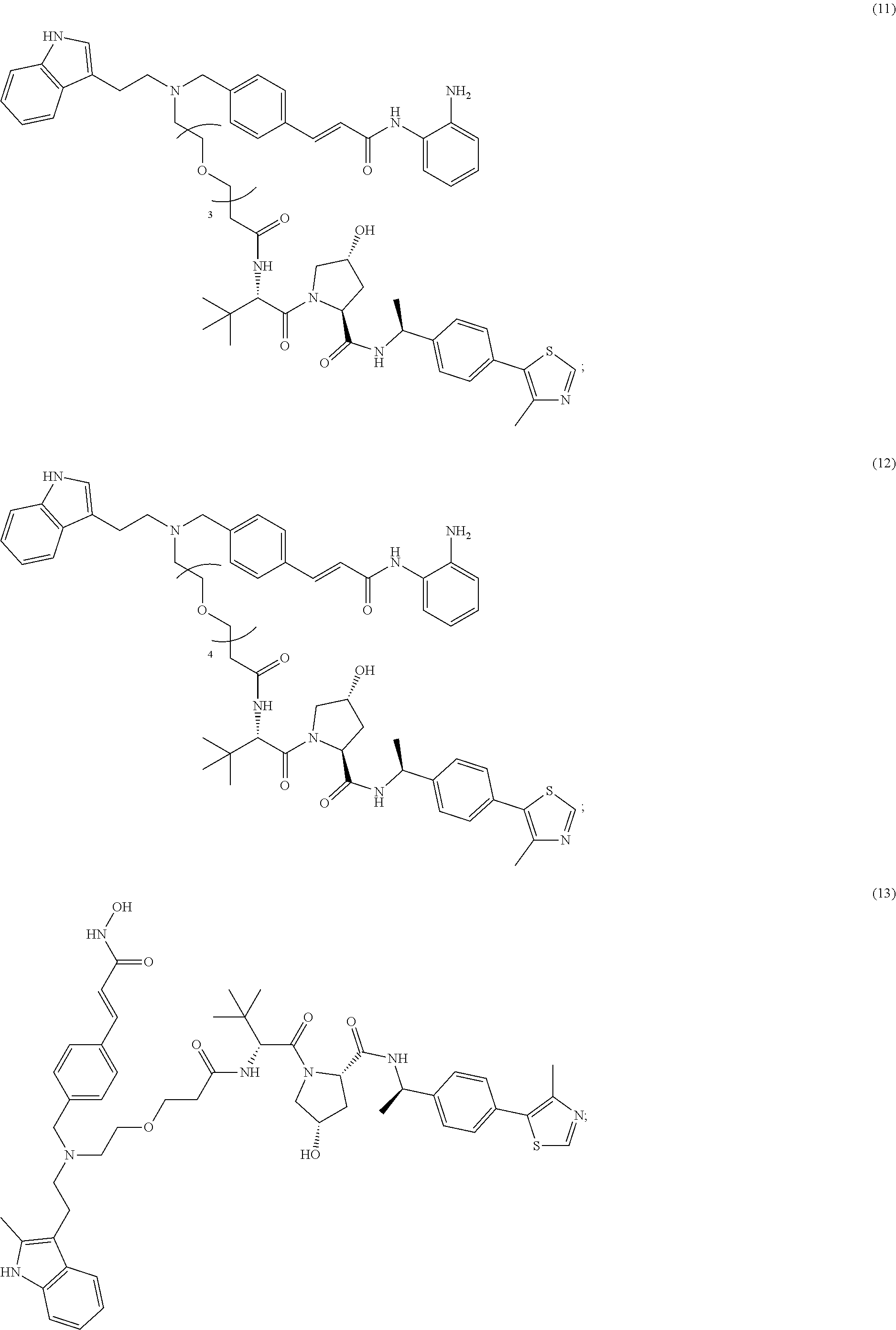

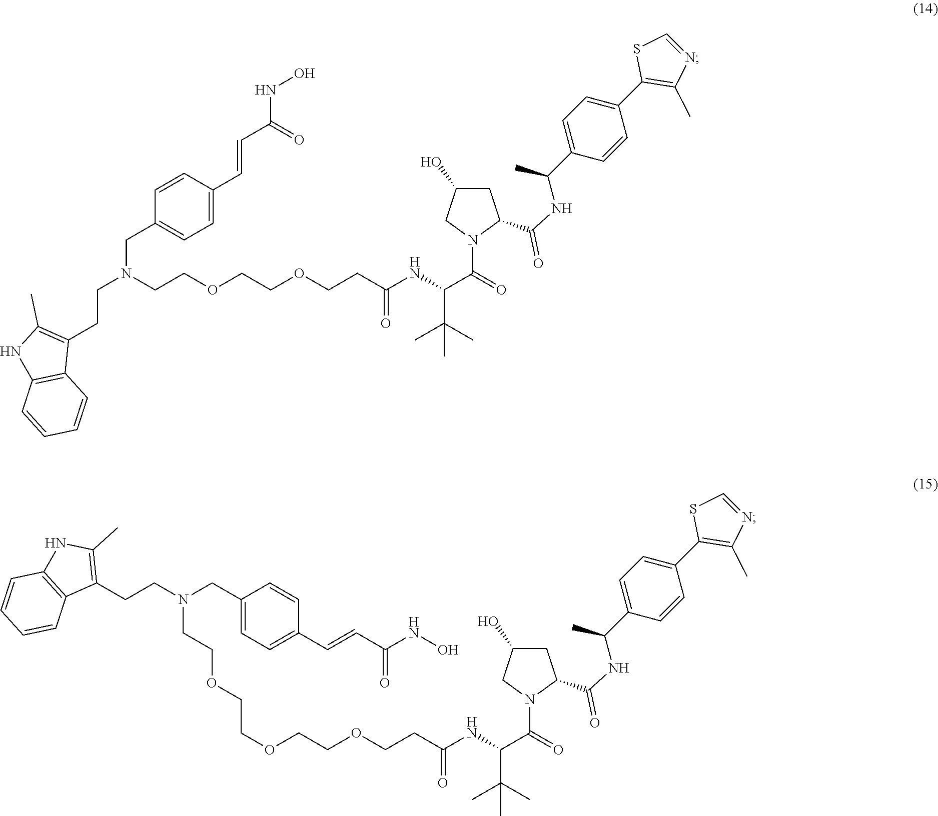 [SMILES: Cc1ncsc1-c1ccc([C@H](C)NC(=O)[C@H]2C[C@@H](O)CN2C(=O)[C@@H](NC(=O)CCOCCOCCN(CCc2c(C)[nH]c3ccccc23)Cc2ccc(/C=C/C(=O)NO)cc2)C(C)(C)C)cc1 ; Cc1ncsc1-c1ccc([C@H](C)NC(=O)[C@H]2C[C@@H](O)CN2C(=O)[C@@H](NC(=O)CCOCCOCCOCCN(CCc2c(C)[nH]c3ccccc23)Cc2ccc(/C=C/C(=O)NO)cc2)C(C)(C)C)cc1]

or a pharmaceutically acceptable salt or stereoisomer thereof.

Bifunctional compounds of formulas (I) and (II) may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

Bifunctional compounds of formulas (I) and (II) may have at least one chiral center. Therefore, they may be in the form of a stereoisomer. As used herein, the term “stereoisomer” embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

In some embodiments, the bifunctional compound of formulas (I) or (II) is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

In addition, bifunctional compounds of formulas (I) and (II) embrace N-oxides, crystalline forms (also known as polymorphs), active metabolites of the compounds having the same type of activity, tautomers, and unsolvated as well as solvated and hydrated forms with pharmaceutically acceptable solvents such as water, ethanol, and the like, of the compounds. The solvated forms of the conjugates presented herein are also considered to be disclosed herein.

### Methods of Synthesis

In some embodiments, the present invention is directed to a method for making bifunctional compounds of formulas (I) and (II) or a pharmaceutically acceptable salt or stereoisomer thereof. Broadly, the inventive compounds or pharmaceutically-acceptable salts or stereoisomers thereof, may be prepared by any process known to be applicable to the preparation of chemically related compounds. The compounds of the present invention will be better understood in connection with the synthetic schemes that described in various working examples that illustrate non-limiting methods by which the compounds of the invention may be prepared.

### Pharmaceutical Compositions

Another aspect of the present invention is directed to a pharmaceutical composition that includes a therapeutically effective amount of an inventive bifunctional compound or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may further include one or more pharmaceutically acceptable excipients.

Broadly, bifunctional compounds of formulas (I) and (II) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the bifunctional compounds are formulated for oral or intravenous administration (e.g., systemic intravenous injection).

Accordingly, bifunctional compounds of the present invention may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bifunctional compounds of the present invention may be formulated in a hard or soft gelatin capsule. Representative excipients that may be used include 44e gelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, bifunctional compounds of formulas (I) and (II) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The bifunctional compounds may be formulated for buccal or sublingual administration, examples of which include tablets, lozenges and gels.

The bifunctional compounds may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bifunctional compounds of formulas (I) and (II) may be formulated for topical administration which as used herein, refers to administration intradermally by application of the formulation to the epidermis. These types of compositions are typically in the form of ointments, pastes, creams, lotions, gels, solutions and sprays.

Representative examples of carriers useful in formulating compositions for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a bifunctional compound of formula (I) or (II) or a pharmaceutically acceptable salt or a stereoisomer thereof; or a composition including a bifunctional compound of formula (I) or (II) or a pharmaceutically acceptable salt or a stereoisomer thereof, effective in producing the desired therapeutic response in a particular patient suffering from a disease or disorder characterized or mediated by aberrant HDAC3 activity. The term “therapeutically effective amount” thus includes the amount of a bifunctional compound of the invention or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., breast cancer, prostate cancer, pancreatic cancer, laryngeal cancer, Hodgkin's lymphoma, neuroblastoma, polycythemia vera, T-cell lymphoma, multiple myeloma, leukemia, hepatocellular carcinoma, non-small cell lung cancer, and essential thrombocythemia) cells, or reduces the amount of HDAC3 in diseased cells.

The total daily dosage of the bifunctional compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the bifunctional compound; and like factors well known in the medical arts (see, for example, Goodman and Gilman's The Pharmacological Basis of Therapeutics, 10th Edition, A. Gilman, J. Hardman and L. Limbird, eds., McGraw-Hill Press, 155-173, 2001).

Bifunctional compounds of formulas (I) and (II) and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of a bifunctional compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present invention is directed to methods of treating diseases or disorders involving aberrant (e.g., dysfunctional or dysregulated) HDAC3 activity, that entails administration of a therapeutically effective amount of a bifunctional compound formula (I) or (II) or a pharmaceutically acceptable salt or stereoisomer thereof, to a subject in need thereof.

The diseases or disorders are characterized or mediated by aberrant HDAC3 activity (e.g., elevated levels of HDAC3, or otherwise functionally abnormal HDAC3 relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

In some embodiments, bifunctional compounds of formulas (I) and (II) may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

Exemplary types of non-cancerous (e.g., cell proliferative) diseases or disorders that may be amenable to treatment with the compounds of the present invention include inflammatory diseases and conditions, autoimmune diseases, neurodegenerative diseases, heart diseases, viral diseases, chronic and acute kidney diseases or injuries, metabolic diseases, and allergic and genetic diseases.

In some embodiments, the bifunctional compounds may be useful in the treatment of neurodegenerative diseases and disorders. As used herein, the term “neurodegenerative diseases and disorders” refers to conditions characterized by progressive degeneration or death of nerve cells, or both, including problems with movement (ataxias), or mental functioning (dementias). Representative examples of such diseases and disorders include Alzheimer's disease (AD) and AD-related dementias, Parkinson's disease (PD) and PD-related dementias, prion disease, motor neuron diseases (MND), Huntington's disease (HD), Pick's syndrome, spinocerebellar ataxia (SCA), spinal muscular atrophy (SMA), primary progressive aphasia (PPA), amyotrophic lateral sclerosis (ALS), traumatic brain injury (TBI), multiple sclerosis (MS), dementias (e.g., vascular dementia (VaD), Lewy body dementia (LBD), semantic dementia, and frontotemporal lobar dementia (FTD).

In some embodiments, the bifunctional compounds may be useful in the treatment of autoimmune diseases and disorders. As used herein, the term “autoimmune disease” refers to conditions where the immune system produces antibodies that attack normal body tissues. Representative examples of such diseases include autoimmune hematological disorders (e.g., hemolytic anemia, aplastic anemia, anhidrotic ectodermal dysplasia, pure red cell anemia and idiopathic thrombocytopenia), Sjogren's syndrome, Hashimoto thyroiditis, rheumatoid arthritis, juvenile (type 1) diabetes, polymyositis, scleroderma, Addison's disease, lupus including systemic lupus erythematosus, vitiligo, pernicious anemia, glomerulonephritis, pulmonary fibrosis, celiac disease, polymyalgia rheumatica, multiple sclerosis, ankylosing spondylitis, alopecia areata, vasculitis, autoimmune uveoretinitis, lichen planus, bullous pemphigus, pemphigus vulgaris, pemphigus foliaceus, paraneoplastic pemphigus, myasthenia gravis, immunoglobulin A nephropathy, Wegener granulomatosis, autoimmune oophoritis, sarcoidosis, rheumatic carditis, ankylosing spondylitis, Grave's disease, autoimmune thrombocytopenic purpura, psoriasis, psoriatic arthritis, dermatitis herpetiformis, ulcerative colitis, and temporal arteritis.

In other embodiments, the methods are directed to treating subjects having cancer. Broadly, the bifunctional compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

Sarcomas that may be treatable with the bifunctional compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumor (mixed connective tissue types), and histiocytic sarcoma (immune cancer).

In some embodiments, methods of the present invention entail treatment of subjects having cell proliferative diseases or disorders of the hematological system, liver, brain, lung, colon, pancreas, prostate, ovary, breast, skin and endometrium.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, a bifunctional compound of the present invention may be used to treat non-metastatic or metastatic lung cancer (e.g., NSCLC, ALK-positive NSCLC, NSCLC harboring ROS1 rearrangement, lung adenocarcinoma, and squamous cell lung carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

In some embodiments, the cancer is breast cancer, prostate cancer, pancreatic cancer, laryngeal cancer, Hodgkin's lymphoma, neuroblastoma, polycythemia vera, T-cell lymphoma, multiple myeloma, leukemia, hepatocellular carcinoma, non-small cell lung cancer, or essential thrombocythemia.

The bifunctional compounds of formulas (I) and (II) may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which were unsuccessful or partially successful but who became intolerant to the particular treatment. Therapy may also be given as an adjuvant treatment, i.e., to prevent reoccurrence of cancer in patients with no currently detectable disease or after surgical removal of a tumor. Thus, in some embodiments, the bifunctional compounds may be administered to a patient who has received another therapy, such as chemotherapy, radioimmunotherapy, surgical therapy, immunotherapy, radiation therapy, targeted therapy or any combination thereof.

The methods of the present invention may entail administration of bifunctional compounds of formulas (I) and (II) or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days) followed by a 7-day “off” period. In other embodiments, the bifunctional compound may be dosed twice a day (BID) over the course of two and a half days (for a total of 5 doses) or once a day (QD) over the course of two days (for a total of 2 doses). In other embodiments, the bifunctional compound may be dosed once a day (QD) over the course of five days.

### Combination Therapy

Bifunctional compounds of formulas (I) and (II) may be used in combination or concurrently with at least one other active agent, e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a bifunctional compound of formula (I) or (II) in combination with one or more additional therapeutics known for use in treating the disease or condition (e.g., cancer). The dosage of the additional anticancer therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference, 60th ed., 2006. For example, anti-cancer agents that may be suitable for use in combination with the inventive bifunctional compounds are known in the art. See, e.g., U.S. Pat. No. 9,101,622 (Section 5.2 thereof) and U.S. Pat. No. 9,345,705 B2 (Columns 12-18 thereof). Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bifunctional antibodies) and CAR-T therapy.

In some embodiments, an inventive bifunctional compound and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

In some embodiments involving cancer treatment, an inventive bifunctional compound and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

### Pharmaceutical Kits

The present bifunctional compounds and/or compositions containing them may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain an inventive bifunctional compound or a pharmaceutical composition thereof. The kits or pharmaceutical systems of the invention may also include printed instructions for using the compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

These and other aspects of the present invention will be further appreciated upon consideration of the following Examples, which are intended to illustrate certain particular embodiments of the invention but are not intended to limit its scope, as defined by the claims.

### Example 1: General Methods

Unless otherwise noted, reagents and solvents were used as received from commercial suppliers. All reactions were monitored using a Waters® Acquity ultra performance liquid chromatography/mass spectrometry (UPLC/MS) system using Acquity UPLC® BEH C18 column (2.1×50 mm, 1.7 μm particle size). UPLC method A: solvent gradient=90% A at 0 min, 5% A at 1.8 min; method B: solvent gradient=85% A at 0 min, 1% A at 1.8 min; solvent A=0.1% formic acid in H₂O; solvent B=0.1% formic acid in acetonitrile; flow rate: 0.6 mL/min. Purification of reaction products was carried out by flash chromatography using CombiFlash®Rf with Teledyne ISCO RediSe® normal-phase silica flash columns; or Waters® high performance liquid chromatography (HPLC) system using SunFirem C18 column (19×100 mm, 5 μm particle size): solvent gradient 0% to 99% acetonitrile in H₂O (0.035% trifluoroacetic acid (TFA) as additive); flow rate: 20 m/min, or SunFirem C18 column (30×250 mm, 5 μm particle size): solvent gradient 0% to 99% acetonitrile in H₂O (0.035% TFA as additive); flow rate: 40 mL/min. The purity of all compounds was over 95% and was analyzed with Waters® UPLC system. ¹H NMR and ¹³C NMR spectra were obtained using Bruker Avance III spectrometers (500 MHz for ¹H, and 125 MHz for ¹³C). Chemical shifts are reported relative to deuterated methanol (δ=3.31) or dimethyl sulfoxide (DMSO) (δ=2.50) for ¹H NMR. Spectra are given in ppm (δ) and as br=broad, s=singlet, d=doublet, t=triplet, q=quartet, m=multiplet and coupling constants (J) are reported in Hertz.

### Example 2: Synthesis of (2S,4R)-1-((S)-2-(3-(2-((2-(1H-indol-3-yl)ethyl)(4-((E)-3-(hydroxyamino)-3-oxoprop-1-en-1-yl)benzyl)amino)ethoxy)propanamido)-3,3-dimethylbutanoyl)-4-hydroxy-N—((S)-1-(4-(4-methylthiazol-5-yl)phenyl)ethyl)pyrrolidine-2-carboxamide (1)

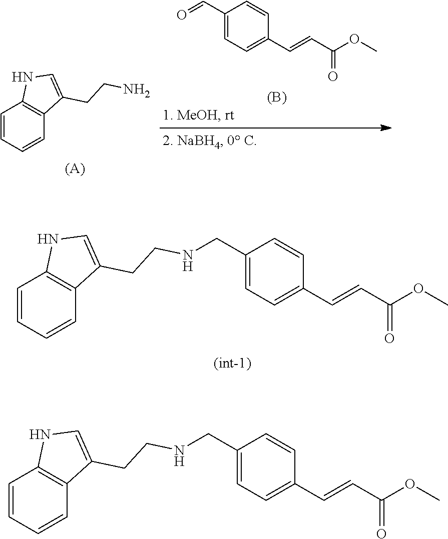

### Methyl (E)-3-(4-(((2-(1H-indol-3-yl)ethyl)amino)methyl)phenyl)acrylate (int-1)

To a solution of tryptamine (A) (1 eq, 1.26 g) in methanol was added methyl (E)-3-(4-formylphenyl)acrylate (B) (1 eq, 1.5 g) at 0° C. The reaction mixture was allowed to warm to room temperature and was stirred for 2 h. The mixture was cooled to 0° C., and NaBH₄ (2 eq, 600 mg) was added in several batches. The mixture was allowed to warm to room temperature and was stirred for an additional 12 h. When the starting material was consumed, the reaction was quenched with aqueous NaHCO₃ and then was extracted with ethyl acetate (three times). The organic layer was combined and washed with brine, dried over Na₂SO₄, filtered and concentrated in vacuo. The residue was purified using ISCO (dichloromethane/methanol, 0%-10%) to yield int-1 (2.9 g).

UPLC-MS RT: 0.80 min (Method A), Mass m/z: (334.97, M+1).

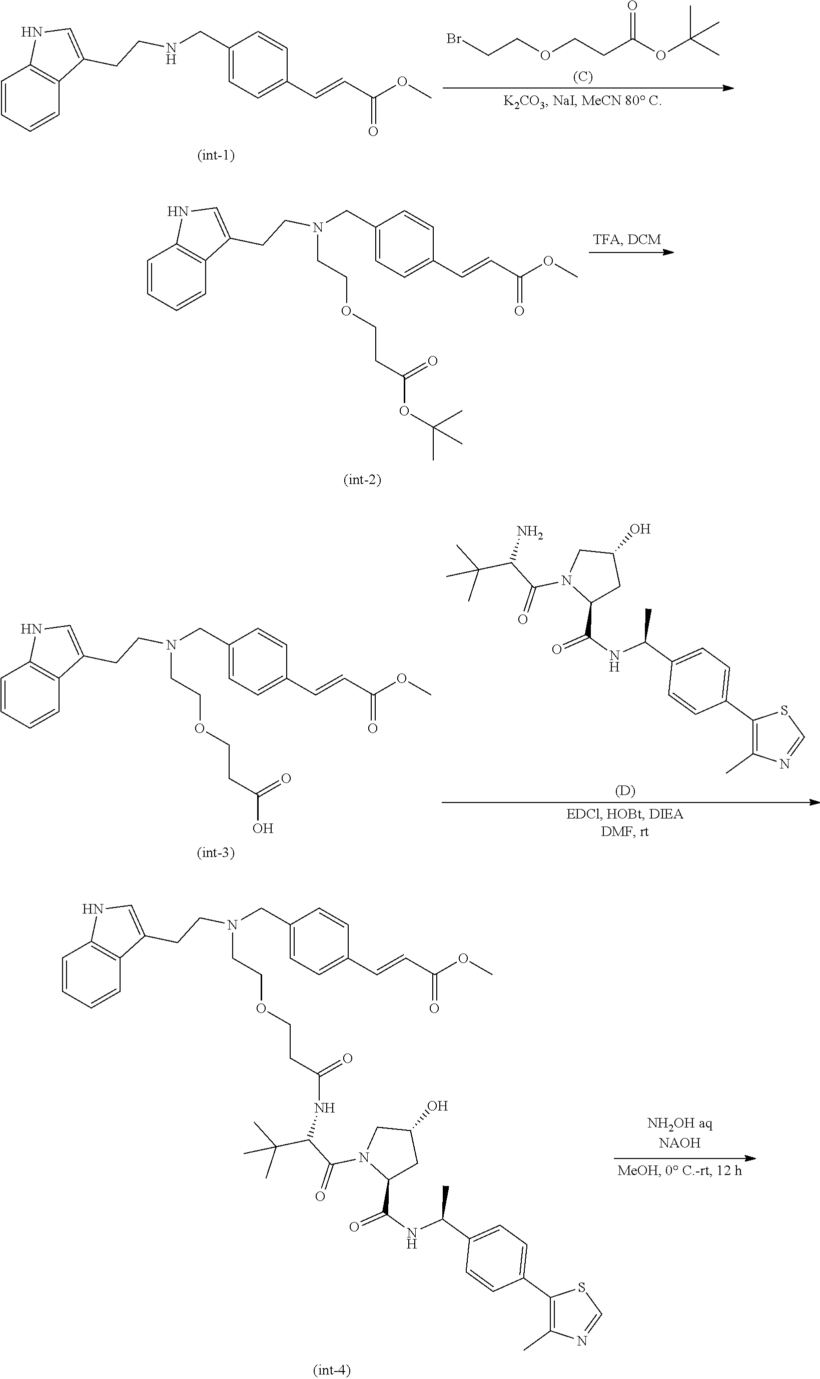

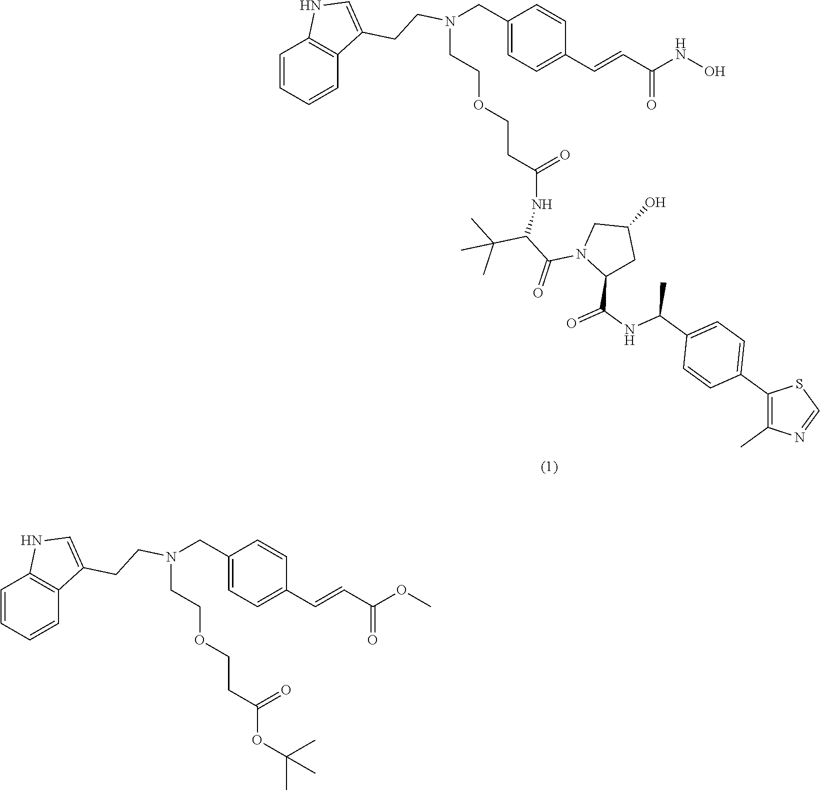

### Methyl (E)-3-(4-(((2-(1H-indol-3-yl)ethyl)(2-(3-(tert-butoxy)-3-oxopropoxy)ethyl)amino)methyl)phenyl)acrylate (int-2)

To a solution of int-1 (1 eq, 75 mg) and tert-butyl 3-(2-bromoethoxy)propanoate (C) (1.3 eq, 87 mg) in acetonitrile (1.2 mL, 0.2 M) was added K₂CO₃ (2 eq, 62 mg) and NaI (0.1 eq, 3.4 mg) in one portion. The reaction mixture was heated to 60° C. and was stirred for 16 h. When the starting material was consumed, the mixture was filtered through a pad of Celite® and concentrated in vacuo. The residue was purified using ISCO (dichloromethane/methanol, 0%-10%) to yield the int-2.

UPLC-MS RT: 1.21 min (Method A), Mass m/z: (506.98, M+1).

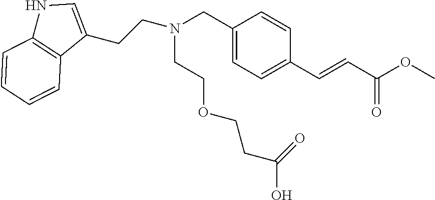 [SMILES: COC(=O)/C=C/c1ccc(CN(CCOCCC(=O)O)CCc2c[nH]c3ccccc23)cc1]

### (E)-3-(2-((2-(1H-Indol-3-yl)ethyl)(4-(3-methoxy-3-oxoprop-1-en-1-yl)benzyl)amino)ethoxy)propanoic acid (int-3)

Int-2 (1 eq) was treated with a mixture of TFA/dichloromethane (1:5), and the reaction was stirred at room temperature for 9 h. When the starting material was consumed, solvent was removed in vacuo, and the residue was used in the next step without further purification.

UPLC-MS RT: 0.94 min (Method A), Mass m/z: (450.98, M+1).

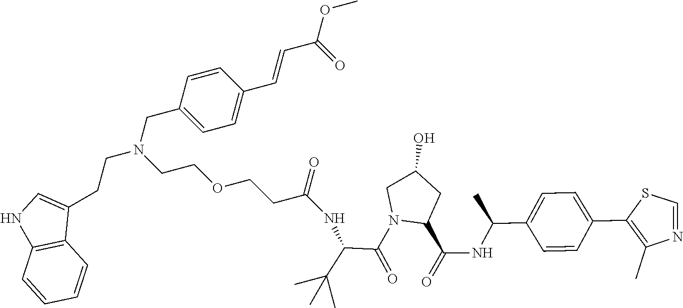

### Methyl (E)-3-(4-(((2-(1H-indol-3-yl)ethyl)(2-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxy)ethyl)amino)methyl)phenyl)acrylate (int-4)

To a solution of int-3 (1 eq) and (2R,4S)-1-((R)-2-amino-3,3-dimethylbutanoyl)-4-hydroxy-N—((R)-1-(4-(4-methylthiazol-5-yl)phenyl)ethyl)pyrrolidine-2-carboxamide (10) (1 eq, 108 mg) in DMF (3 mL), was added 1-ethyl-3-(3-dimethylaminopropyl)carbodiimide (EDCI) (1.2 eq, 51.6 mg), hydroxybenzotriazole (HOBt) (1.2 eq, 36.4 mg) and N, N-diisopropylethylamine (DIEA) (3 eq, 120 μL). The reaction mixture was stirred at room temperature for 7 h. When the starting material was consumed, solvent was removed in vacuo, and the residue was purified using HPLC (H₂O/acetonitrile, 0%-100%) to yield the title compound (59 mg, 30% over 3 steps).

UPLC-MS RT: 1.32 min (Method A), Mass m/z: (876.72, M+1).

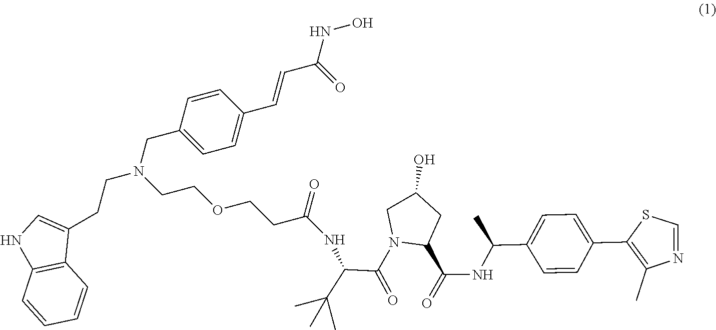

A solution of int-4 (1 eq, 32 mg) in methanol (1 mL, 0.05 M) was treated with 50 wt % aq. NH₂OH (10 eq, 22 μL) followed by 25 wt % NaOMe in methanol (5 eq, 42 μL) at 0° C. The reaction mixture was gradually warmed to room temperature and stirred for 2 h. When the starting material was consumed, solvent was removed in vacuo, and the residue was purified using HPLC (H₂O/acetonitrile, 0%-100%) to yield compound 1 as a white solid (9.9 mg, 31%).

UPLC-MS RT: 0.98 min (Method A), Mass m/z: (877.62, M+1).

### Example 3: Synthesis of (2S,4R)-1-((S)-14-(tert-butyl)-3-(4-((E)-3-(hydroxyamino)-3-oxoprop-1-en-1-yl)benzyl)-1-(1H-indol-3-yl)-12-oxo-6,9-dioxa-3,13-diazapentadecan-15-oyl)-4-hydroxy-N—((S)-1-(4-(4-methylthiazol-5-yl)phenyl)ethyl)pyrrolidine-2-carboxamide (2)

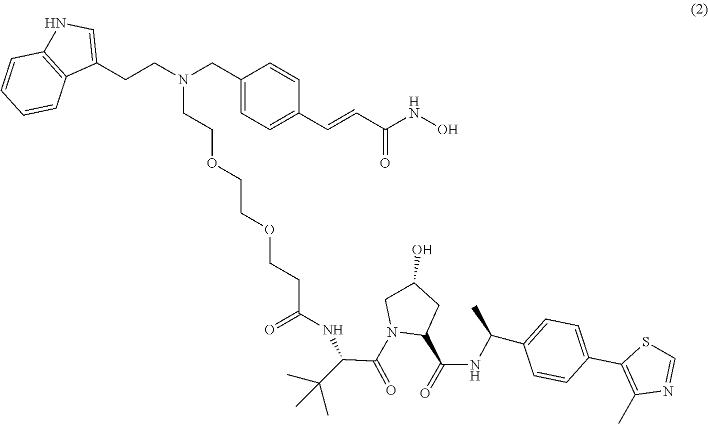

Compound 2 was prepared in an analogous manner to compound 1 in Example 2 from int-1 and tert-butyl 3-(2-(2-bromoethoxy)ethoxy)propanoate.

¹H NMR (500 MHz, DMSO-d₆, as a TFA salt) δ 10.96 (s, 1H), 10.82 (s, 1H), 9.69 (s, 1H), 9.15 (s, 1H), 8.98 (s, 1H), 8.36 (d, J=7.8 Hz, 1H), 7.85 (d, J=9.3 Hz, 1H), 7.67 (d, J=8.0 Hz, 2H), 7.62 (d, J=7.8 Hz, 2H), 7.49 (d, J=15.8 Hz, 1H), 7.45-7.40 (m, 3H), 7.39-7.33 (m, 3H), 7.21 (s, 1H), 7.08 (t, J=7.0 Hz, OH), 6.97 (t, J=7.4 Hz, 1H), 6.53 (d, J=15.9 Hz, 1H), 5.12 (s, 1H), 4.90 (p, J=7.2 Hz, 1H), 4.59-4.45 (m, 3H), 4.41 (t, J=8.1 Hz, 1H), 4.28 (s, 1H), 3.90-3.75 (m, 2H), 3.72-3.41 (m, 8H), 3.43-3.27 (m, 4H), 3.25-3.07 (m, 2H), 2.55-2.47 (m, 1H), 2.45 (s, 3H), 2.33 (dt, J=14.8, 6.1 Hz, 1H), 2.07-1.97 (m, 1H), 1.79 (ddd, J=12.9, 8.7, 4.6 Hz, 1H), 1.36 (d, J=7.0 Hz, 3H), 0.91 (s, 9H).

UPLC-MS RT: 0.81 min (Method B), Mass m/z: (922.59, M+1).

### Example 4: Synthesis of (2S,4R)-1-((S)-17-(tert-butyl)-3-(4-((E)-3-(hydroxyamino)-3-oxoprop-1-en-1-yl)benzyl)-1-(1H-indol-3-yl)-15-oxo-6,9,12-trioxa-3,16-diazaoctadecan-18-oyl)-4-hydroxy-N—((S)-1-(4-(4-methylthiazol-5-yl)phenyl)ethyl)pyrrolidine-2-carboxamide

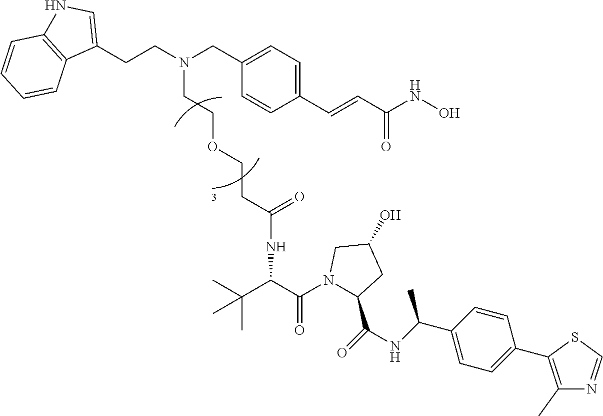

Compound 3 was prepared in an analogous manner to compound 1 in Example 2.

¹H NMR (400 MHz, DMSO-d₆) δ (ppm) 13.11-12.53 (m, 2H), 10.75 (s, 3H), 9.02 (d, J=24.1 Hz, 2H), 8.40 (d, J=7.9 Hz, 1H), 8.14 (s, 1H), 7.88 (d, J=8.9 Hz, 1H), 7.80-7.23 (m, 6H), 7.04 (dd, J=55.3, 28.0 Hz, 4H), 6.46 (s, 1H), 5.12 (d, J=3.2 Hz, 1H), 4.92 (d, J=6.9 Hz, 1H), 4.61-4.17 (m, 3H), 3.73 (s, 1H), 3.41 (d, J=50.8 Hz, 14H), 2.83 (s, 1H), 2.59 (d, J=64.7 Hz, 6H), 2.45 (s, 3H), 2.31 (d, J=17.1 Hz, 1H), 2.3 (s, 1H), 2.01 (s, 1H), 1.78 (s, 1H), 1.37 (d, J=6.9 Hz, 3H), 0.92 (s, 9H).

LCMS (m/z): RT: 1.5 min, Mass m/z: 483.9 [M/2+H]⁺.

### Example 5: Synthesis of (2S,4R)-1-((S)-17-(tert-butyl)-3-(4-((E)-3-(hydroxyamino)-3-oxoprop-1-en-1-yl)benzyl)-1-(1H-indol-3-yl)-15-oxo-6,9,12-trioxa-3,16-diazaoctadecan-18-oyl)-4-hydroxy-N—((S)-1-(4-(4-methylthiazol-5-yl)phenyl)ethyl)pyrrolidine-2-carboxamide (4)

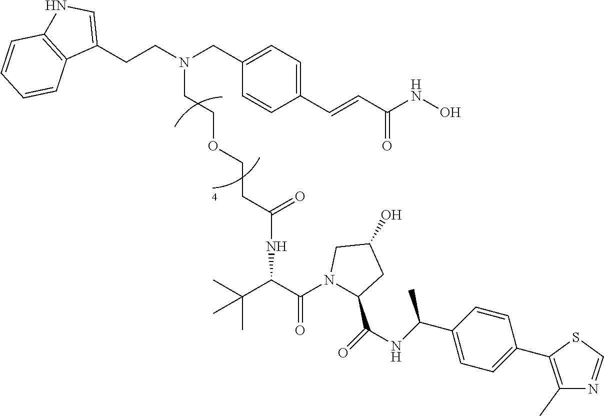

Compound 4 was prepared in an analogous manner to compound 1 in Example 2.

¹H NMR (400 MHz, DMSO-d₆) δ (ppm) 11.39 (s, 1H), 10.98 (s, 1H), 8.99 (s, 3H), 8.40 (d, J=7.8 Hz, 1H), 7.89 (d, J=9.4 Hz, 1H), 7.67 (d, J=7.4 Hz, 2H), 7.53 (t, J=12.0 Hz, 4H), 7.41 (dd, J=23.9, 8.4 Hz, 5H), 7.30-7.20 (m, 1H), 7.11 (dd, J=13.8, 6.6 Hz, 1H), 7.01 (t, J=7.4 Hz, 1H), 6.49 (d, J=15.8 Hz, 1H), 4.97-4.84 (m, 1H), 4.58-4.39 (m, 2H), 4.26 (d, J=14.3 Hz, 4H), 3.98 (s, 2H), 3.52 (s, 18H), 3.20 (s, 2H), 3.12-3.01 (m, 2H), 2.55 (dd, J=13.8, 6.2 Hz, 1H), 2.46 (s, 3H), 2.36 (dd, J=13.2, 7.0 Hz, 1H), 2.08-1.94 (m, 1H), 1.79 (ddd, J=12.9, 8.7, 4.6 Hz, 1H), 1.42 (dd, J=37.8, 6.9 Hz, 3H), 0.94 (s, 9H).

LCMS (m/z): RT: 1.06 min, m/z: 505.9 [M/2+H]⁺.

### Example 6: Synthesis of (2S,4R)-1-((S)-23-(tert-butyl)-3-(4-((E)-3-(hydroxyamino)-3-oxoprop-1-en-1-yl)benzyl)-1-(1H-indol-3-yl)-21-oxo-6,9,12,15,18-pentaoxa-3,22-diazatetracosan-24-oyl)-4-hydroxy-N—((S)-1-(4-(4-methylthiazol-5-yl)phenyl)ethyl)pyrrolidine-2-carboxamide (5)

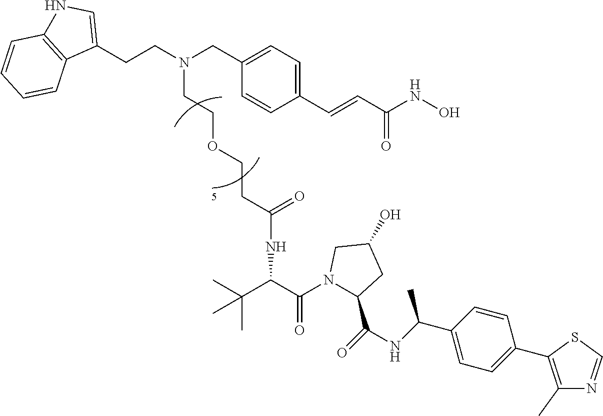

Compound 5 was prepared in an analogous manner to compound 1 in Example 2.

¹H NMR (400 MHz, DMSO-d₆) δ (ppm) 11.45 (s, 1H), 11.04 (s, 1H), 9.05 (s, 3H), 8.46 (d, J=7.8 Hz, 1H), 7.94 (d, J=9.2 Hz, 1H), 7.83-7.53 (m, 6H), 7.45 (dt, J=20.1, 9.9 Hz, 5H), 7.34-7.23 (m, 1H), 7.17 (dd, J=14.7, 7.5 Hz, 1H), 7.06 (dd, J=16.0, 9.0 Hz, 1H), 6.58 (t, J=17.1 Hz, 1H), 4.96 (dd, J=14.3, 7.0 Hz, 1H), 4.64-4.47 (m, 2H), 4.32 (d, J=14.8 Hz, 3H), 4.04 (s, 2H), 3.61 (ddd, J=28.8, 16.5, 9.8 Hz, 22H), 3.27 (s, 2H), 3.18-3.08 (m, 2H), 2.61 (dd, J=13.5, 6.0 Hz, 1H), 2.52 (s, 3H), 2.45-2.36 (m, 1H), 2.12-2.04 (m, 1H), 1.85 (ddd, J=12.8, 8.5, 4.6 Hz, 1H), 1.48 (dd, J=37.2, 7.0 Hz, 3H), 1.00 (s, 9H).

LCMS (m/z): RT: 1.06 min, m/z: 527.8 [M/2+H]⁺.

### Example 7: Synthesis of (2S,4R)—N-(2-((5-((2-(1H-indol-3-yl)ethyl)(4-((E)-3-(hydroxyamino)-3-oxoprop-1-en-1-yl)benzyl)amino)pentyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (7)

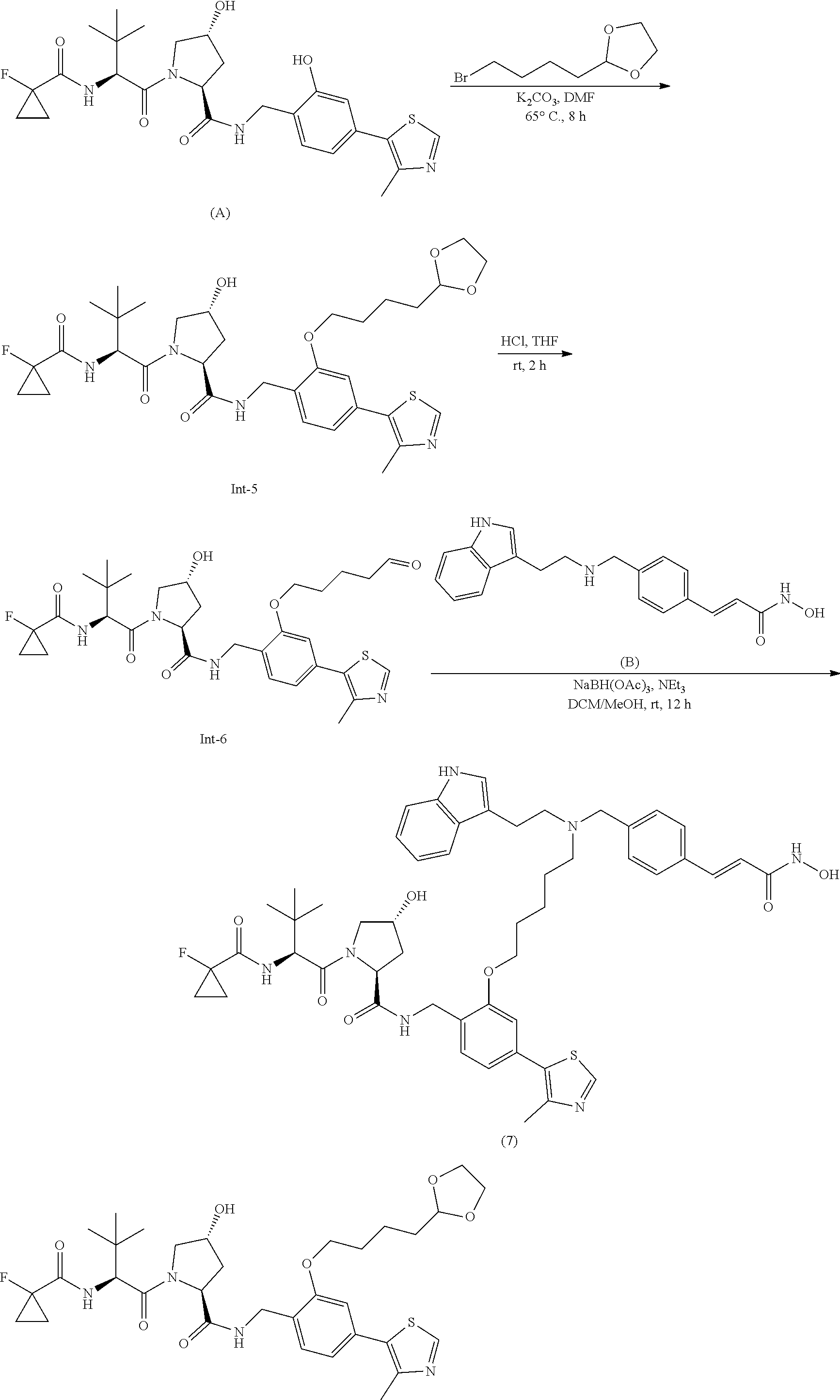

### (2S,4R)—N-(2-(4-(1,3-Dioxolan-2-yl)butoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (int-5)

To a solution of compound A (Zoppi et al., J. Med. Chem. 62:699-726 (2019)) (1 eq, 150 mg) and 2-(2-bromoethyl)-1,3-dioxolane (1.2 eq, 71 mg) in DMF (1.5 mL, 0.19 M) was added with K₂CO₃ (1.5 eq, 58 mg). The reaction mixture heated to 65° C. and stirred for 4 h. The reaction was monitored by UPLC-MS. Upon consumption of the starting material, the reaction was filtered, extracted three times with EtOAc, dried over Na₂SO₄, filtered, and concentrated in vacuo. The resulting product was purified using ISCO (DCM/MeOH) to yield int-5 (187 mg, quant. yield).

UPLC-MS RT: 1.28 min (Method A), Mass m/z: 661.50 (M+1).

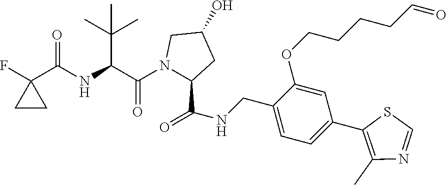 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCC=O)c1]

### (2S,4R)-1-((S)-2-(1-Fluorocyclopropane-1-carboxamido)-3,3-dimethylbutanoyl)-4-hydroxy-N-(4-(4-methylthiazol-5-yl)-2-((5-oxopentyl)oxy)benzyl)pyrrolidine-2-carboxamide (int-6)

Int-5 (1 eq, 30 mg) was treated with a 1:2 mixture of HCl in THF (0.75 mL) at room temperature. The reaction was stirred for 2 h, and monitored by UPLC-MS. Upon consumption of the starting material, the reaction was quenched with aqueous NaHCO₃, extracted three times with EtOAc, dried over Na₂SO₄, filtered, and concentrated in vacuo. The crude product was used in the next step without further purification.

UPLC-MS RT: 1.15 min (Method A), Mass m/z: 617.29 (M+1).

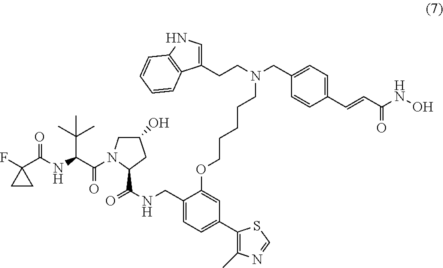 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCN(CCc2c[nH]c3ccccc23)Cc2ccc(/C=C/C(=O)NO)cc2)c1]

Int-6 (1 eq, crude from last step) and compound B (Shult (1 eq, 30 mg) was dissolved in a solvent mixture of DCM/MeOH (2 mL/10 drops, 0.045 M), the reaction mixture was treated with NEt3 (4 eq, 48 μL) and NaBH(OAc)₃ (2 eq, 40 mg) at room temperature, The reaction was stirred for 12 h, and monitored by UPLC-MS. Upon comsumption of the starting material, the reaction was quenched with aqueous NaHCO₃, extracted three times with EtOAc, dried over Na₂SO₄, filtered, and concentrated in vacuo. The crude product was purified using HPLC (H2O/acetonitrile, 0%-100%) to yield compound 7 (27 mg, 32% yield over 2 steps).

UPLC-MS RT: 1.45 min (Method A), Mass m/z: 936.62 (M+1).

### Example 8: Synthesis of (2S,4R)—N-(2-(2-(2-(2-((2-(1H-indol-3-yl)ethyl)(4-((E)-3-(hydroxyamino)-3-oxoprop-1-en-1-yl)benzyl)amino)ethoxy)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (8)

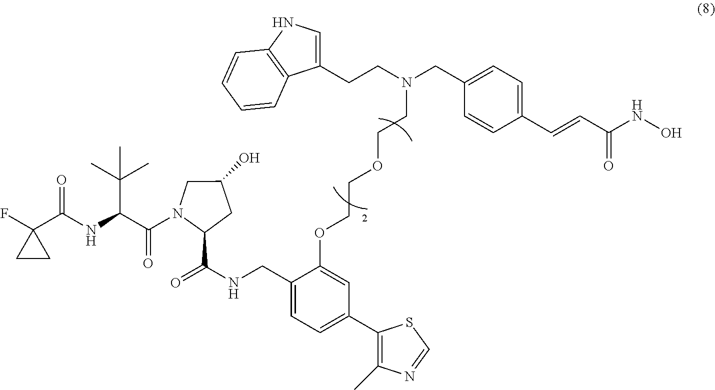

Compound 8 was prepared in an analogous manner to compound 7 in Example 7.

UPLC-MS RT: 1.25 min (Method A), Mass m/z: 981.83 (M+1).

### Example 9: Synthesis of (2S,4R)-1-((S)-2-(3-(2-((2-(1H-indol-3-yl)ethyl)(4-((E)-3-((2-aminophenyl)amino)-3-oxoprop-1-en-1-yl)benzyl)amino)ethoxy)propanamido)-3,3-dimethylbutanoyl)-4-hydroxy-N—((S)-1-(4-(4-methylthiazol-5-yl)phenyl)ethyl)pyrrolidine-2-carboxamide (9)

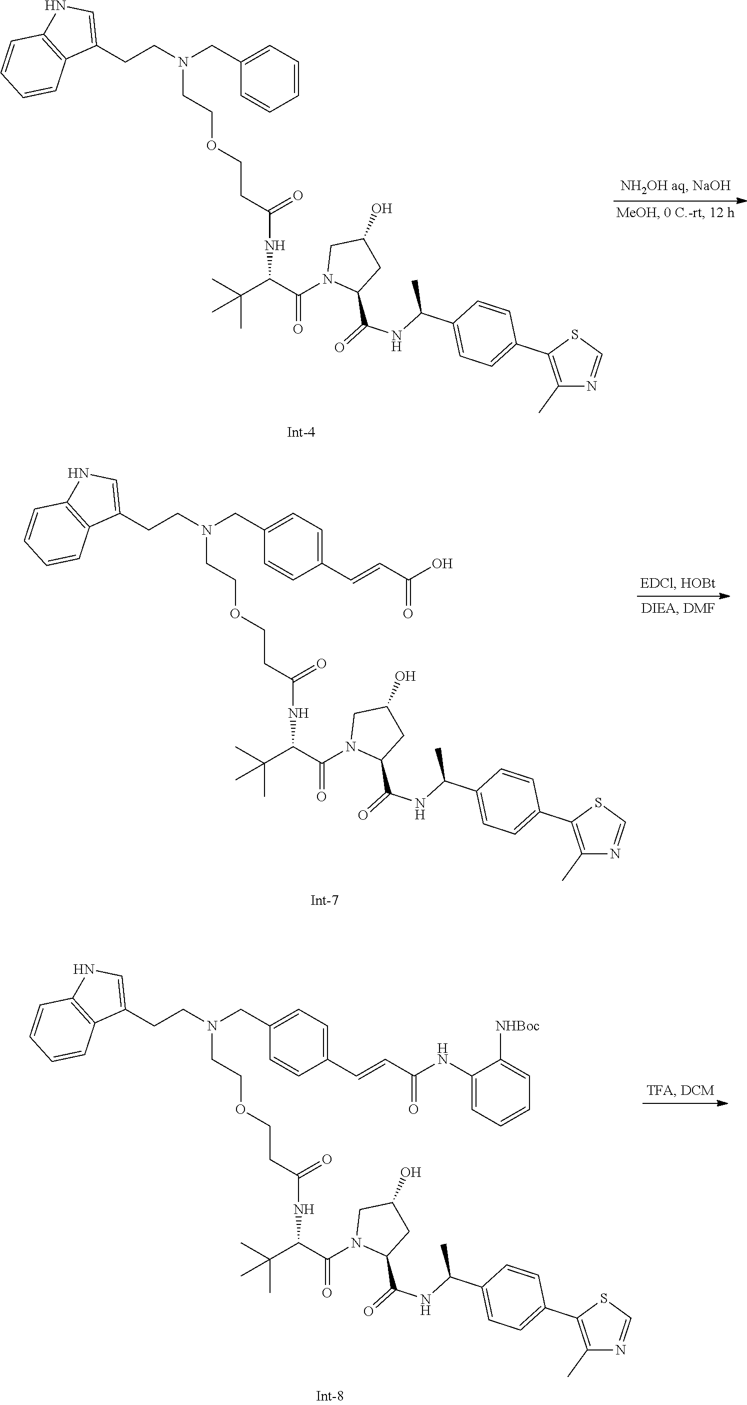 [SMILES: Cc1ncsc1-c1ccc([C@H](C)NC(=O)[C@@H]2C[C@@H](O)CN2C(=O)[C@@H](NC(=O)CCOCCN(CCc2c[nH]c3ccccc23)Cc2ccccc2)C(C)(C)C)cc1 ; Cc1ncsc1-c1ccc([C@H](C)NC(=O)[C@@H]2C[C@@H](O)CN2C(=O)[C@@H](NC(=O)CCOCCN(CCc2c[nH]c3ccccc23)Cc2ccc(/C=C/C(=O)O)cc2)C(C)(C)C)cc1 ; Cc1ncsc1-c1ccc([C@H](C)NC(=O)[C@@H]2C[C@@H](O)CN2C(=O)[C@@H](NC(=O)CCOCCN(CCc2c[nH]c3ccccc23)Cc2ccc(/C=C/C(=O)Nc3ccccc3NC(=O)OC(C)(C)C)cc2)C(C)(C)C)cc1]

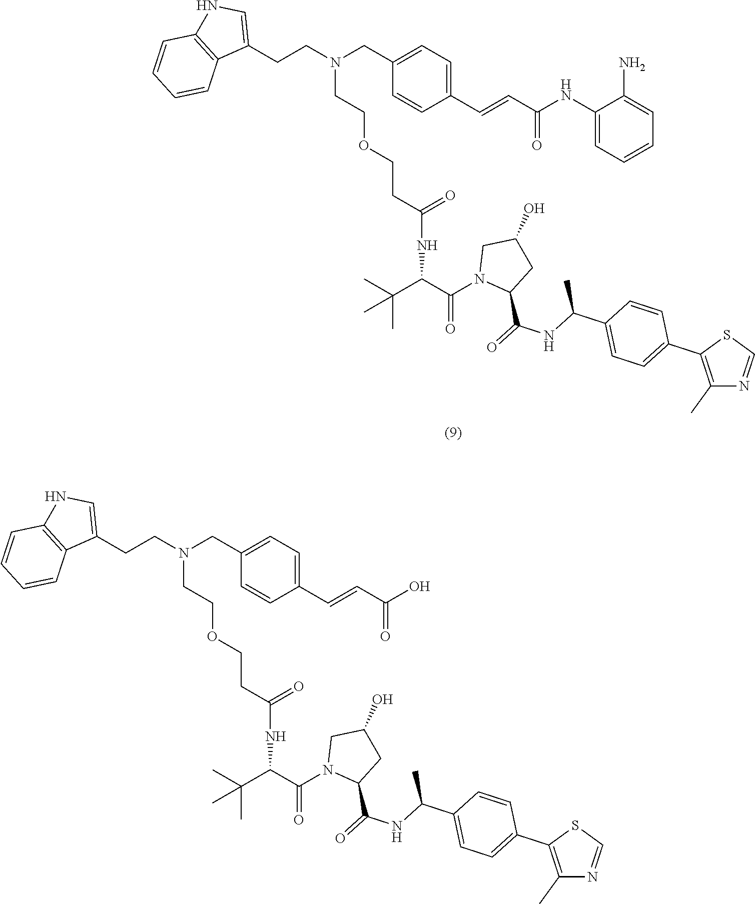 [SMILES: Cc1ncsc1-c1ccc([C@H](C)NC(=O)[C@@H]2C[C@@H](O)CN2C(=O)[C@@H](NC(=O)CCOCCN(CCc2c[nH]c3ccccc23)Cc2ccc(/C=C/C(=O)Nc3ccccc3N)cc2)C(C)(C)C)cc1 ; Cc1ncsc1-c1ccc([C@H](C)NC(=O)[C@@H]2C[C@@H](O)CN2C(=O)[C@@H](NC(=O)CCOCCN(CCc2c[nH]c3ccccc23)Cc2ccc(/C=C/C(=O)O)cc2)C(C)(C)C)cc1]

### (E)-3-(4-(((2-(1H-Indol-3-yl)ethyl)(2-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxy)ethyl)amino)methyl)phenyl)acrylic acid (int-7)

A solution of int-4 (1 eq, 32 mg) in methanol (1 mL, 0.05 M) was treated with 50 wt % aq. NH₂OH (10 eq, 22 μL) followed by 25 wt % o NaOMe in methanol (5 eq, 42 μL) at 0° C. The reaction mixture was gradually warmed to room temperature and stirred for 2 h. Upon consumption of the starting material, solvent was removed in vacuo, and the residue was purified using HPLC (H₂O/acetonitrile, 0%-100%) to yield int-7 as a white solid (10 mg, 32% o).

UPLC-MS RT: 1.04 min (Method A), Mass m/z: 862.72 (M+1).

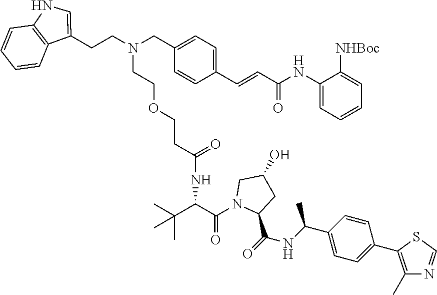 [SMILES: Cc1ncsc1-c1ccc([C@H](C)NC(=O)[C@@H]2C[C@@H](O)CN2C(=O)[C@@H](NC(=O)CCOCCN(CCc2c[nH]c3ccccc23)Cc2ccc(/C=C/C(=O)Nc3ccccc3NC(=O)OC(C)(C)C)cc2)C(C)(C)C)cc1]

### tert-Butyl (2-((E)-3-(4-(((2-(1H-indol-3-yl)ethyl)(2-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxy)ethyl)amino)methyl)phenyl)acrylamido)phenyl)carbamate (int-8)

To a solution of int-5 (1 eq, 10 mg) and tert-butyl (2-aminophenyl)carbamate (1.5 eq, 3.7 mg) in DMF (0.5 mL), was added EDCI (1.2 eq, 2.7 mg), HOBt (1.2 eq, 1.9 mg) and DIEA (2 eq, 4 μL). The reaction mixture was stirred at room temperature for 24 h and monitored by UPLC-MS. Upon comsumption of the starting material, the reaction was quenched with H₂O, extracted with EtOAc, dried over Na₂SO₄, filtered, and concentrated in vacuo. The resulting residue was purified using ISCO (DCM/MeOH) to yield int-8.

UPLC-MS RT: 1.43 min (Method A), Mass m/z: 1052.64 (M+1).

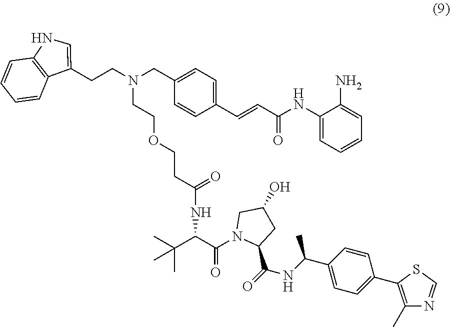 [SMILES: Cc1ncsc1-c1ccc([C@H](C)NC(=O)[C@@H]2C[C@@H](O)CN2C(=O)[C@@H](NC(=O)CCOCCN(CCc2c[nH]c3ccccc23)Cc2ccc(/C=C/C(=O)Nc3ccccc3N)cc2)C(C)(C)C)cc1]

Int-6 was treated with a mixture of TFA/DCM. The reaction was stirred for 3 h, and monitored by UPLC-MS. Upon comsumption of the starting material, the reaction was concentrated in vacuo and purified using HPLC (H₂O/acetonitrile, 0%-100%) to yield compound 9 (1.3 mg, 12% yield over 2 steps).

UPLC-MS ORT: 1.17 min (Method A), Mass m/z: 952.73 (M+1).

### Example 10: Synthesis of 2S,4R)-1-((S)-3-(4-((E)-3-((2-aminophenyl)amino)-3-oxoprop-1-en-1-yl)benzyl)-14-(tert-butyl)-1-(1H-indol-3-yl)-12-oxo-6,9-dioxa-3,13-diazapentadecan-15-oyl)-4-hydroxy-N—((S)-1-(4-(4-methylthiazol-5-yl)phenyl)ethyl)pyrrolidine-2-carboxamide (10)

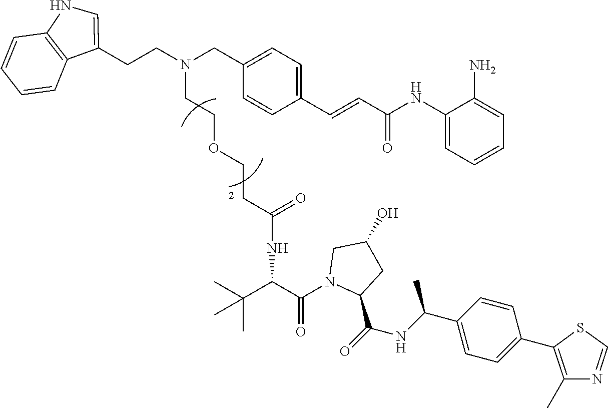

Compound 10 was prepared in an analogous manner to compound 9 in Example 9.

UPLC-MS RT: 1.20 min (Method A), Mass m/z: 996.83 (M+1).

### Example 11: Synthesis of (2S,4R)-1-((S)-3-(4-((E)-3-((2-aminophenyl)amino)-3-oxoprop-1-en-1-yl)benzyl)-17-(tert-butyl)-1-(1H-indol-3-yl)-15-oxo-6,9,12-trioxa-3,16-diazaoctadecan-18-oyl)-4-hydroxy-N—((S)-1-(4-(4-methylthiazol-5-yl)phenyl)ethyl)pyrrolidine-2-carboxamide (11)

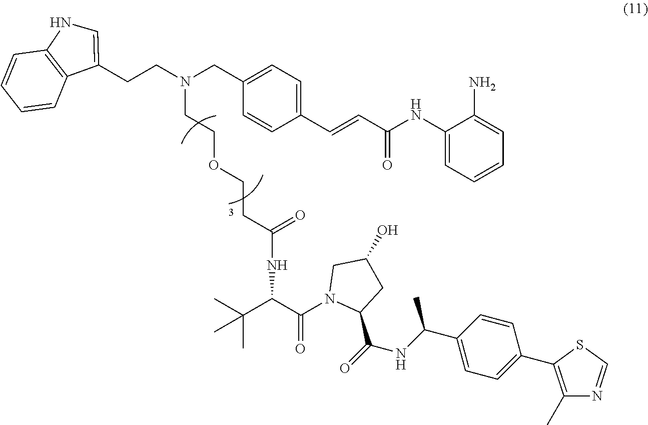

Compound 11 was prepared in an analogous manner to compound 9 in Example 9.

¹H NMR (400 MHz, CD₃OD) δ (ppm) 8.80 (s, 2H), 7.64 (dd, J=28.2, 11.8 Hz, 4H), 7.45 (d, J=8.0 Hz, 2H), 7.40-7.05 (m, 11H), 7.05-6.99 (m, 1H), 6.88 (t, J=13.3 Hz, 2H), 4.48 (dd, J=18.2, 10.4 Hz, 3H), 4.27 (d, J=32.9 Hz, 1H), 3.85-3.69 (m, 3H), 3.68-3.60 (m, 2H), 3.56-3.42 (m, 11H), 2.52-2.23 (m, 6H), 2.15-2.05 (m, 1H), 1.84 (s, 1H), 1.41 (dd, J=31.3, 7.0 Hz, 3H), 1.21 (d, J=18.1 Hz, 2H), 0.87 (dd, J=35.7, 14.7 Hz, 12H).

LCMS: 521.9 [M/2+H]⁺.

### Example 12: Synthesis of (2S,4R)-1-((S)-3-(4-((E)-3-((2-aminophenyl)amino)-3-oxoprop-1-en-1-yl)benzyl)-20-(tert-butyl)-1-(1H-indol-3-yl)-18-oxo-6,9,12,15-tetraoxa-3,19-diazahenicosan-21-oyl)-4-hydroxy-N—((S)-1-(4-(4-methylthiazol-5-yl)phenyl)ethyl)pyrrolidine-2-carboxamide (12)

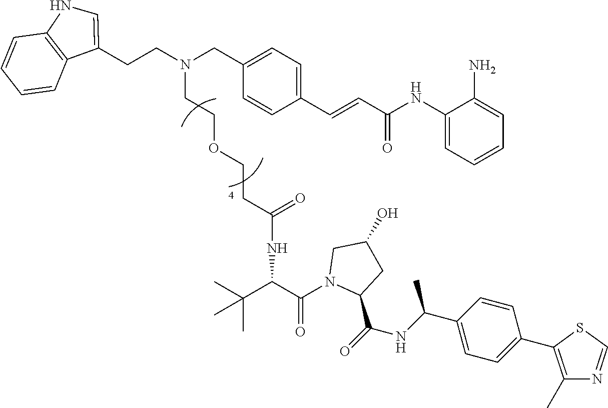

Compound 12 was prepared in an analogous manner to compound 9 in Example 9.

¹H NMR (400 MHz, CD₃OD) δ (ppm) 9.76 (s, 2H), 7.73 (d, J=15.6 Hz, 1H), 7.61 (s, 2H), 7.36 (ddd, J=34.4, 24.7, 14.9 Hz, 13H), 7.13 (s, 1H), 7.02 (s, 1H), 6.90 (d, J=14.6 Hz, 2H), 5.25 (s, 1H), 4.90 (d, J=5.7 Hz, 3H), 4.58-4.42 (m, 4H), 4.31 (s, 1H), 3.83-3.72 (m, 3H), 3.58-3.43 (m, 12H), 2.49 (s, 3H), 2.36 (d, J=30.3 Hz, 2H), 2.10 (d, J=10.7 Hz, 1H), 1.87 (d, J=42.1 Hz, 2H), 1.56-1.45 (m, 2H), 1.39 (d, J=6.5 Hz, 3H), 1.23 (s, 3H), 0.92 (s, 9H), 0.80 (s, 2H).

LCMS (m/z): 543.4 [M/2+H]⁺.

### Example 13: Biochemical Profiling

Compounds 2 and 9 were tested in an enzymatic assay from Reaction Biology® against HDAC isoforms. IC₅₀ was derived from a 10-dose response curve (Tables 1 and 2).

HDAC Assay Protocol

### Reagents

Base Reaction buffer: 50 mM Tris-HCl, pH8.0, 137 mM NaCl, 2.7 mM KCl, and 1 mM MgCl₂.
Add fresh: 1 mg/ml BSA, 1% DMSO

### Substrate

### Fluorogenic HDAC General Substrate (HDAC1, HDAC2, HDAC3, HDAC6, HDAC10, and HDAC11): 50 μM, Arg-His-Lys-Lys(Ac)-AMC

(HDAC8 only substrate: 50 μM, Arg-His-Lys(Ac)-Lys(Ac))-AMC

### Class 2A Substrate (HDAC4, HDAC5, HDAC7, and HDAC9): Boc-Lys(trifluoroacetyl)-AMC

For SIRTs 1-3, general Class 1 HDAC substrate and 500 μM NAD+

### For SIRT5, Ac-Lys-succ and 500 μM NAD+

Reaction Procedure

### Deacetylation Step

1. Delivered 2X enzyme in wells of reaction plate except in the control wells and added buffer to control wells.
2. Delivered compounds in 100% DMSO into the enzyme mixture by Acoustic technology (Echo550; nanoliter range). Spin down and pre-incubation.
3. Delivered 2X Substrate Mixture (Fluorogenic HDAC Substrate and co-factor if applicable) in all reaction wells to initiate the reaction. Spin and shake.
4. Incubated for 30 min for Class 2A, 1 h for HDAC1, 2, 3, and 6, and 2 h for the rest of HDAC isoforms and SIRTs at 30° C. with seal.

### Development Step

5. Added Developer with Trichostatin A (or Nicotinamide for SIRTs) to stop the reaction and to generate fluorescent color.
6. The fluorescence that was generated was detected with excitation (Ex) at 360 nM and emission (Em) at 460 nM by the EnVision Multilabel Plate Reader (PerkinElmer®, Santa Clara, Calif., USA).

IC₅₀ values were derived from a 10-dose response curve. As shown in Tables 1-2, the addition of a linker and CRBN targeting group altered the isoform selectivity of the SAHA-based degrader compounds.

TABLE 1
Biochemical selectivity of compound 2.

| | Compound IC₅₀ (M) | |
|---|---|---|
| Target | SAHA¹ | Compound 2 |
| HDAC1 | 3.06E−07 | 4.81E−07 |
| HDAC2 | 2.42E−07 | 1.03E−06 |
| HDAC3 | 1.32E−07 | 5.77E−07 |
| HDAC6 | 1.98E−08 | 7.85E−08 |
| HDAC8 | 3.06E−07 | 1.16E−06 |
| HDAC5 | 2.72E−05 | 2.69E−06 |
| HDAC4 | 7.60E−05 | 1.31E−05 |
| HDAC7 | 1.05E−04 | 1.71E−05 |
| HDAC9 | 1.41E−04 | >2.00E−05 |
| HDAC10 | 4.32E−07 | NT³ |

¹from Reaction Biology ®
³ NT, not tested

The biochemical selectivity data summarized in Table 1 show that inventive bifunctional compound 2 potently inhibited HDAC6 activity in vitro. Moderate inhibition of deacetylase activity was observed for HDACs 1, 2, 3, and 8 with inventive bifunctional compound 2.

TABLE 2
Biochemical selectivity of compound 9.

| | Compound IC₅₀ (M) |
|---|---|
| Target | Compound 9 |
| HDAC1 | 9.50E−06 |
| HDAC2 | 1.15E−05 |
| HDAC3 | 1.12E−05 |
| HDAC4 | NT |
| HDAC5 | NT |
| HDAC6 | — |
| HDAC7 | NT |
| HDAC8 | — |
| HDAC9 | NT |

NT, not tested
—, not active

The biochemical selectivity data summarized in Table 2 show that inventive bifunctional compound 9 demonstrated moderate inhibition towards HDACs 1, 2 and 3. Compound 9 did not inhibit HDAC6 or HDAC8 in vitro.

### Example 14: Analysis of Change to Cellular Protein Abundance in Response to Treatment with Inventive Compounds

Lysis buffer (8 M Urea, 50 mM NaCl, 50 mM 4-(2hydroxyethyl)-1-piperazineethanesulfonic acid (EPPS) pH 8.5, protease and phosphatase inhibitors from Roche®) were added to the cell pellets and homogenized by 20 passes through a 21 gauge (1.25 in. long) needle to achieve a cell lysate with a protein concentration between 1-4 mg mL-1. A micro-BCA assay (Pierce™) was used to determine the final protein concentration in the cell lysate. 200 μg of protein for each sample were reduced and alkylated as described in Donovan et al., Elife 7:e38430 (2018).

Proteins were precipitated using methanol/chloroform. Four volumes of methanol were added to the cell lysate, followed by one volume of chloroform, and finally three volumes of water. The mixture was vortexed and centrifuged to separate the chloroform phase from the aqueous phase. The precipitated protein was washed with three volumes of methanol, centrifuged and the resulting washed precipitated protein was allowed to air dry. The precipitated protein was resuspended in 4 M Urea, 50 mM HEPES pH 7.4, followed by dilution to 1 M urea with the addition of 200 mM EPPS, pH 8. Proteins were first digested with LysC (1:50; enzyme:protein) for 12 hours at room temperature. The LysC digestion was diluted to 0.5 M Urea with 200 mM EPPS pH 8 followed by digestion with trypsin (1:50; enzyme:protein) for 6 hours at 37° C. Tandem mass tag (TMT) reagents (Thermo Fisher Scientific) were dissolved in anhydrous acetonitrile (ACN) according to manufacturer's instructions.

Anhydrous ACN was added to each peptide sample to a final concentration of 30% v/v, and labeling was induced with the addition of TMT reagent to each sample at a ratio of 1:4 peptide:TMT label. The 10-plex labeling reactions were performed for 1.5 hours at room temperature and the reaction quenched by the addition of hydroxylamine to a final concentration of 0.3% for 15 minutes at room temperature. The sample channels were combined at a 1:1:1:1:1:1:1:1:1:1:1 ratio, desalted using Cis solid phase extraction cartridges (Waters®) and analyzed by LC-MS for channel ratio comparison. Samples were then combined using the adjusted volumes determined in the channel ratio analysis and dried down in a speed vacuum. The combined sample was then resuspended in 1% formic acid, and acidified (pH 2-3) before being subjected to desalting with C18 SPE (Sep-Pak®, Waters®). Samples were then offline fractionated into 96 fractions by high pH reverse-phase HPLC (Agilent® LC1260) through an aeris peptide xb-c18 column (Phenomenex®) with mobile phase A containing 5% acetonitrile and 10 mM NH₄HCO₃ in LC-MS grade H₂O, and mobile phase B containing 90% acetonitrile and 10 mM NH₄HCO₃ in LC-MS grade H₂O (both pH 8.0). The 96 resulting fractions were then pooled in a non-continuous manner into 24 fractions and these fractions were used for subsequent mass spectrometry analysis.

Data were collected using an Orbitrap Fusion™ Lumos™ mass spectrometer (Thermo Fisher Scientific, San Jose, Calif., USA) coupled with a Proxeon EASY-nLC™ 1200 LC pump (Thermo Fisher Scientific). Peptides were separated on an EasySpray™ ES803 75 μm inner diameter microcapillary column (ThermoFisher Scientific). Peptides were separated using a 190 min gradient of 6-27% acetonitrile in 1.0% formic acid with a flow rate of 350 nL/min.

Each analysis used an MS3-based TMT method as described previously. The data were acquired using a mass range of m z 340-1350, resolution 120,000, automatic gain control (AGC) target 1×10⁶, maximum injection time 100 ms, dynamic exclusion of 120 seconds for the peptide measurements in the Orbitrap Fusion™ Lumos™ mass spectrometer. Data dependent MS2 spectra were acquired in the ion trap with a normalized collision energy (NCE) set at 55%, AGC target set to 1.5×10⁵ and a maximum injection time of 150 ms. MS3 scans were acquired in the Orbitrap Fusion™ Lumos™ mass spectrometer with a higher energy collision dissociation (HCD) set to 55%, AGC target set to 1.5×10⁵, maximum injection time of 150 ms, resolution at 50,000 and with a maximum synchronous precursor selection (SPS) precursors set to 10.

Proteome Discoverer 2.2 (Thermo Fisher Scientific) was used for .RAW file processing and controlling peptide and protein level false discovery rates, assembling proteins from peptides, and protein quantification from peptides. MS/MS spectra were searched against a Uniprot human database (September 2016) with both the forward and reverse sequences. Database search criteria are as follows: tryptic with two missed cleavages, a precursor mass tolerance of 20 ppm, fragment ion mass tolerance of 0.6 Da, static alkylation of cysteine (57.02146 Da), static TMT labelling of lysine residues and N-termini of peptides (229.16293 Da), and variable oxidation of methionine (15.99491 Da). TMT reporter ion intensities were measured using a 0.003 Da window around the theoretical m z for each reporter ion in the MS3 scan. Peptide spectral matches with poor quality MS3 spectra were excluded from quantitation (summed signal-to-noise across 10 channels<200 and precursor isolation specificity<0.5), and resulting data was filtered to only include proteins that had a minimum of 2 unique peptides identified. Reporter ion intensities were normalized and scaled using in-house scripts in the R framework. Statistical analysis was carried out using the limma package within the R framework.

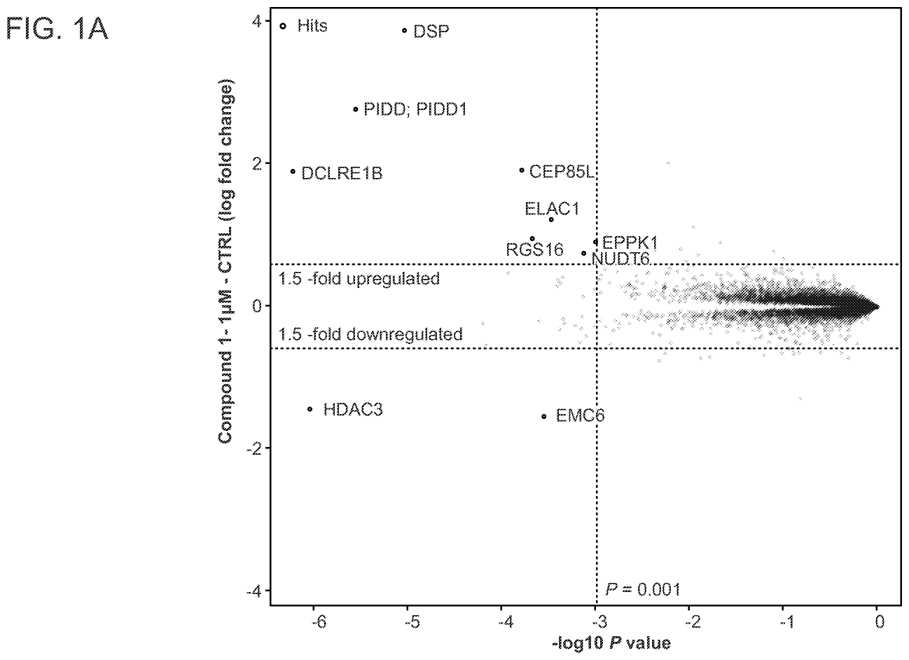

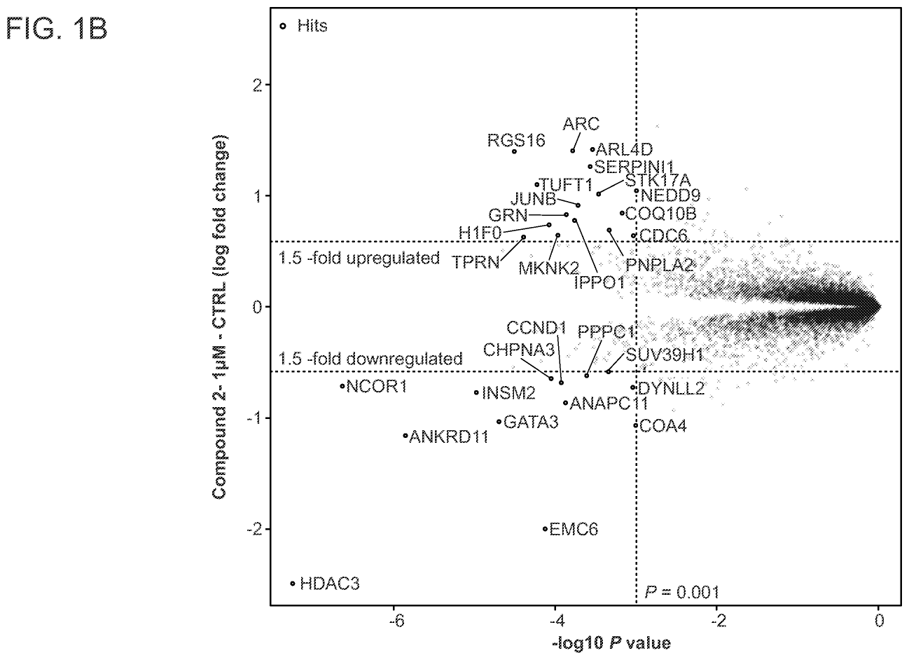

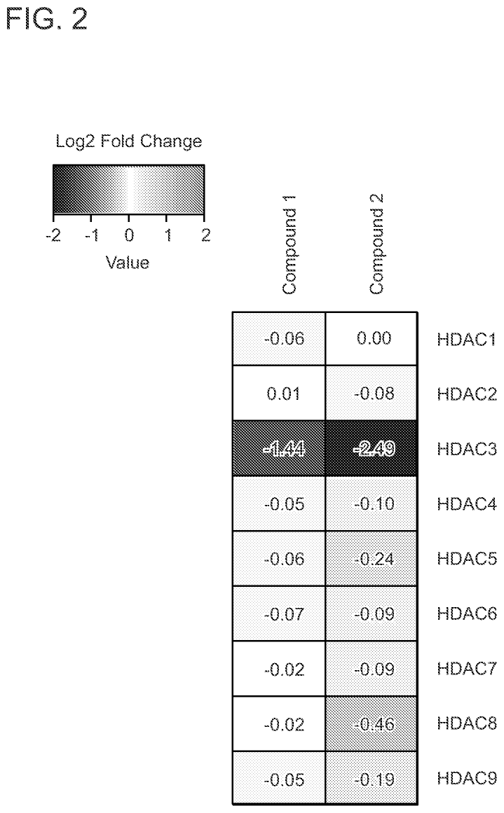

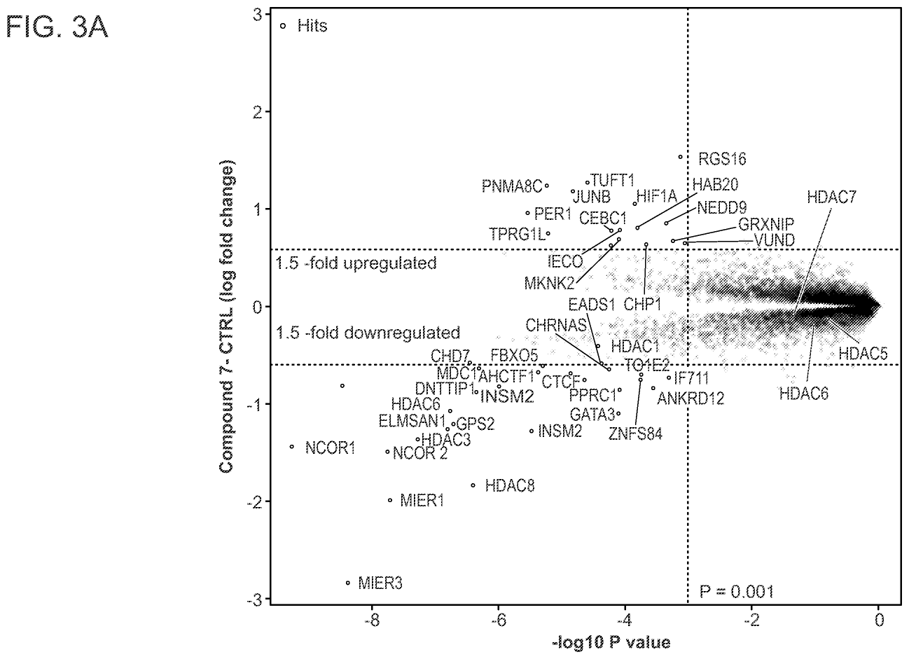

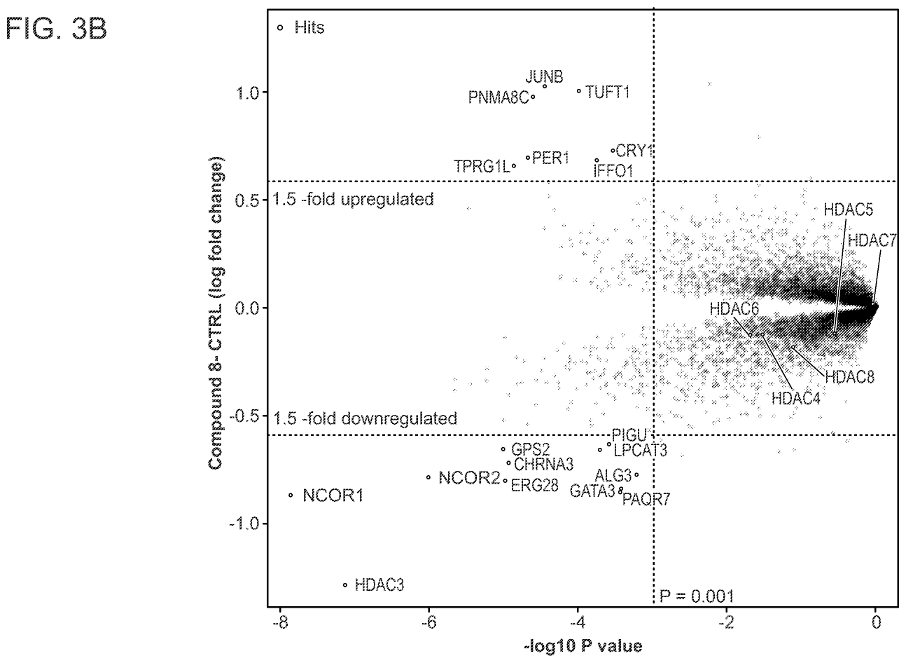

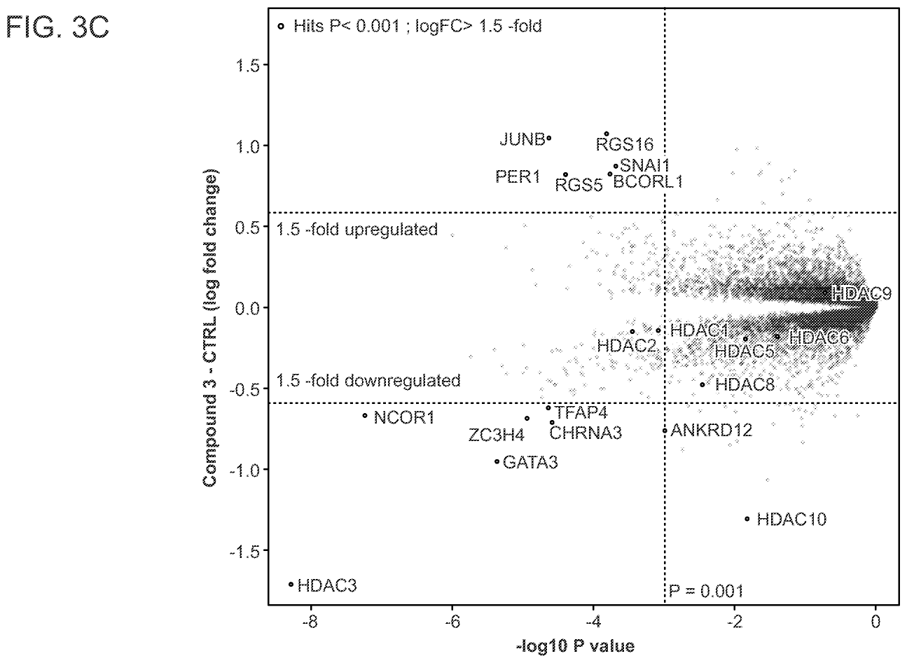

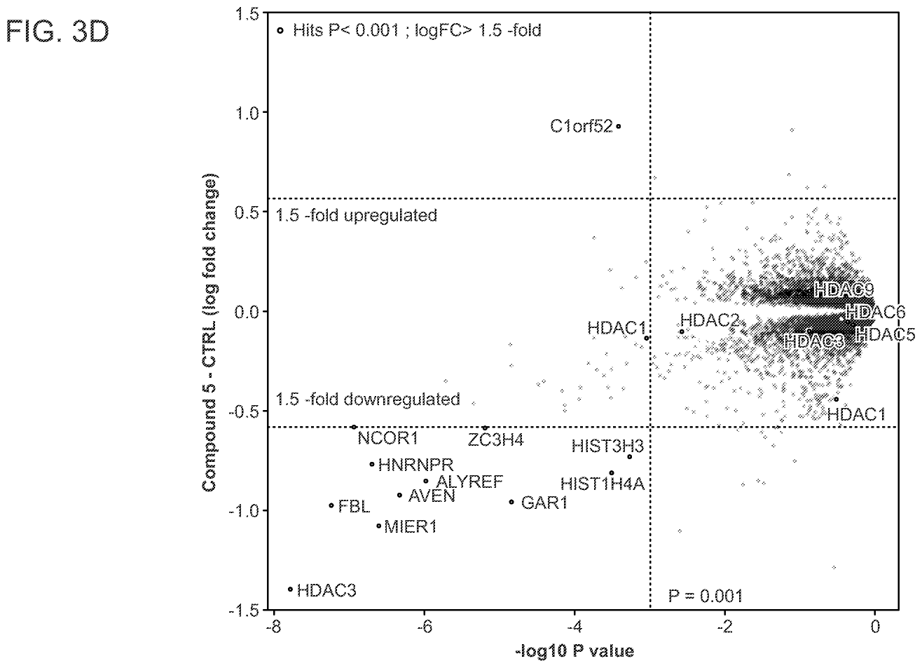

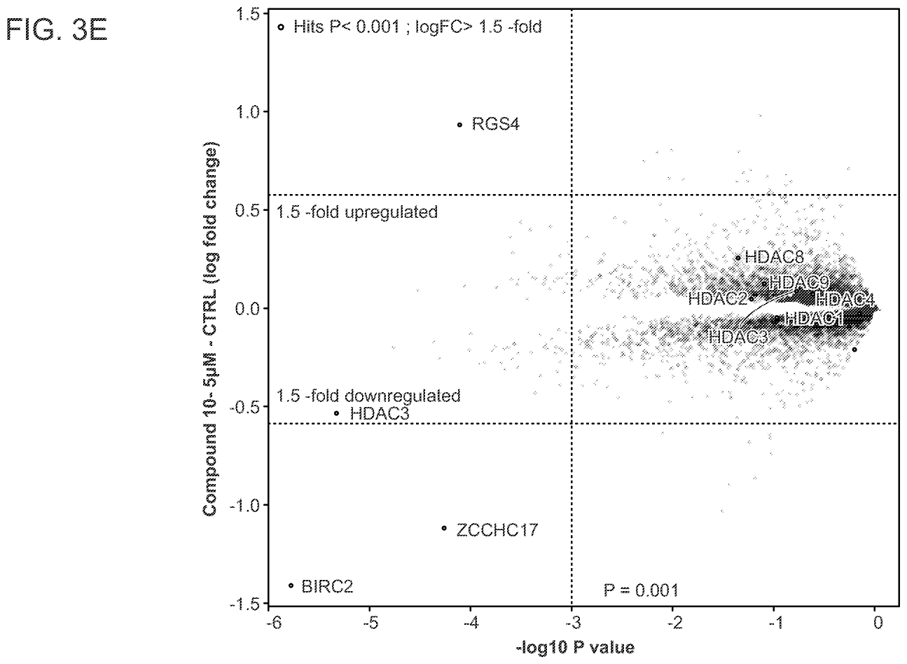

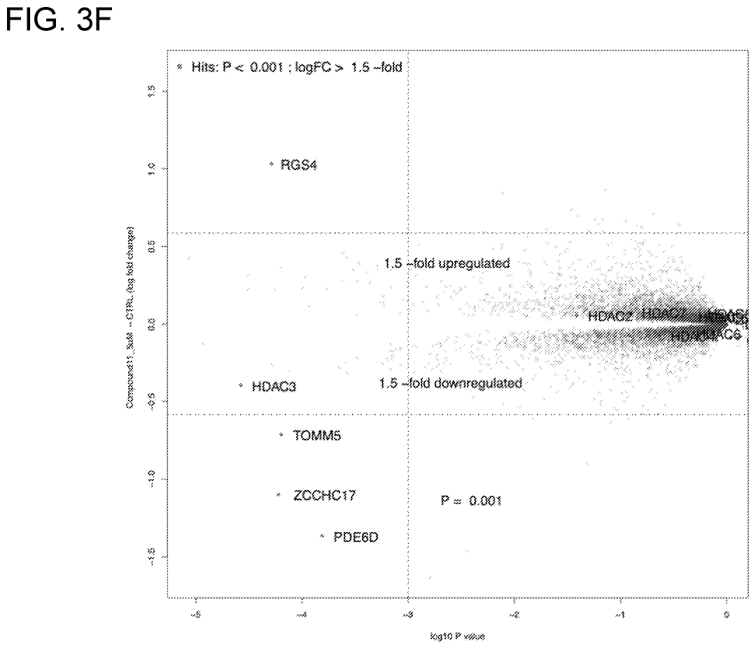

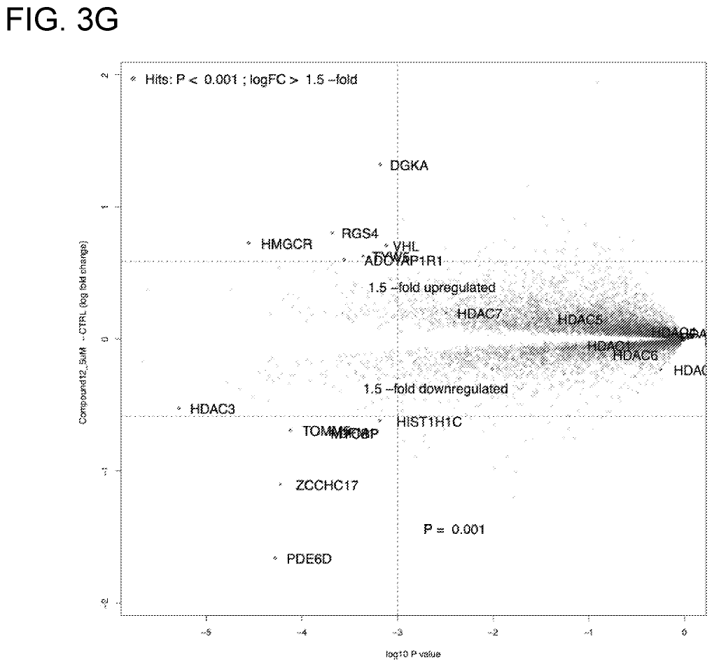

The results are summarized in FIG. 1A-FIG. 1B, FIG. 2, and FIG. 3A-FIG. 3G.

The scatterplots in FIG. 1A-FIG. 1B show the relative change in relative protein abundance with treatment of Kelly cells with inventive bifunctional compounds 1 (FIG. 1A) and 2 (FIG. 1B) compared to dimethyl sulfoxide (DMSO) control. Significant changes were assessed by moderated t-test and displayed with log 2 fold change on the y-axis and negative log₁₀ P values on the x-axis for one independent biological replicate of inventive compound and three independent biological replicates of DMSO. As shown, treatment with each of inventive bifunctional compounds 1 and 2 induced a significant and selective reduction in HDAC3 protein levels when compared to the DMSO treated cells.

The heat map in FIG. 2 displays the change in relative protein abundance of HDAC's identified in the experiment with treatment of Kelly cells with inventive bifunctional compounds 1 and 2 (1 μM, 5 hours), compared to DMSO control. Significant changes were assessed by moderated t-test and colored according to movement and the log 2 fold change value displayed in the box for one independent biological replicates of treatment and three independent biological replicates of DMSO. The results illustrated in FIG. 2 show that inventive bifunctional compounds 1 and 2 induced down-regulation of cellular HDAC3 protein levels, but did not affect the cellular protein levels of the other identified HDAC proteins.

The scatterplots in FIG. 3A-FIG. 3G show the relative change in relative protein abundance with treatment of Kelly cells with inventive bifunctional compounds 7 (FIG. 3A), 8 (FIG. 3B), 3 (FIG. 3C), 5 (FIG. 3D), 10 (FIG. 3E), 11 (FIG. 3F), and 12 (FIG. 3G) compared to dimethyl sulfoxide (DMSO) control. Significant changes were assessed by moderated t-test and displayed with log 2 fold change on the y-axis and negative log₁₀ P values on the x-axis for one independent biological replicate of inventive compound and three independent biological replicates of DMSO. As shown, treatment with inventive bifunctional compound 7 induced a significant and selective reduction in HDAC3/6/8 protein levels when compared to the DMSO treated cells. Treatment with inventive bifunctional compounds 3, 5, 8, and 10-12 induced a significant and selective reduction in HDAC3 protein levels when compared to the DMSO treated cells.

### Example 15: Cellular VHL Engagement Assay

Cells stably expressing the BRD4BD2-GFP with mCherry reporter (40) were seeded at 30-50% confluency in 384-well plates with 50 μL per well of FluoroBrite™ DMEM media (Thermo Fisher Scientific, A18967) supplemented with 10% FBS a day before compound treatment. Compounds and 250 nM AT1((2S,4R)-1-(2R)-2-Acetamido-3-[[6-[2-[(6S)-4-(4-chlorophenyl)-2,3,9-trimethyl-6H-thieno[3,2-f][1,2,4]triazolo[4,3-a][1,4]diazepin-6-yl]acetamido]hexyl]thio]-3-methylbutanoyl]-4-hydroxy-N-[4-(4-methylthiazol-5-yl)benzyl)pyrrolidinine-2-carboxamide) were dispensed using a D300e Digital Dispenser (HP), normalized to 0.5% DMSO, and incubated with the cells for 5 h. The assay plate was imaged immediately using an Acumen® High Content Imager (TTP Labtech) with 488 nm and 561 nm lasers in a 2 μm×1 μm grid per well format. The resulting images were analyzed using CellProfiler™.

A series of image analysis steps (an ‘image analysis pipeline’) was constructed. First, the red and green channels were aligned and cropped to target the middle of each well (to avoid analysis of the heavily clumped cells at the edges). A background illumination function was calculated for both red and green channels of each well individually and subtracted to correct for illumination variations across the 384-well plate from various sources of error. An additional step was then applied to the green channel to suppress the analysis of large auto fluorescent artifacts and enhance the analysis of cell specific fluorescence by way of selecting for objects under a given size (30 A.U.) and with a given shape (speckles). mCherry-positive cells were then identified in the red channel by filtering for objects 8-60 pixels in diameter and by using intensity to distinguish between clumped objects. The green channel was then segmented into GFP positive and negative areas and objects were labeled as GFP positive if at least 40% of it overlapped with a GFP positive area. The fraction of GFP-positive cells/mCherry-positive cells in each well was then calculated, and the green and red images were rescaled for visualization. The values for the concentrations that lead to a 50% increase in BRD4BD2-eGFP accumulation (EC₅₀) were calculated using the nonlinear fit variable slope model (GraphPad Software).

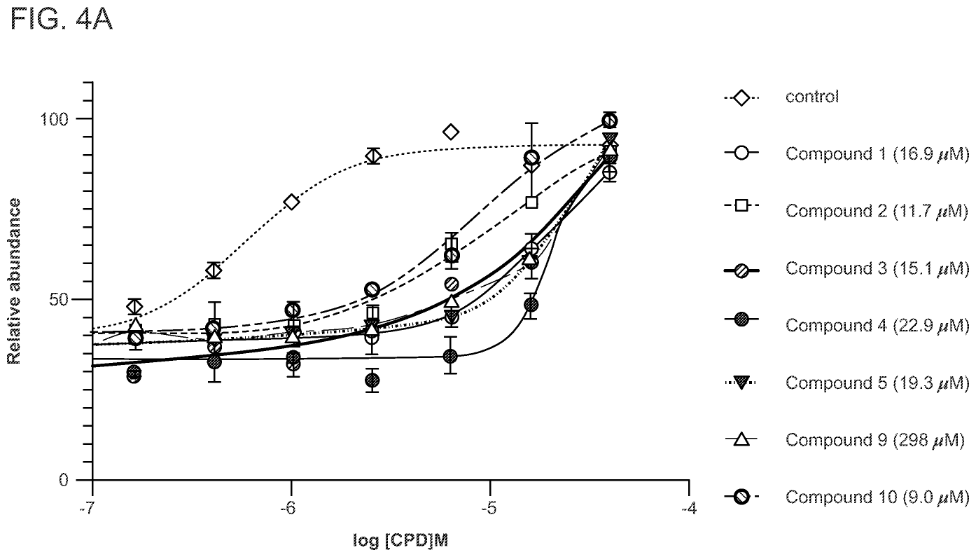

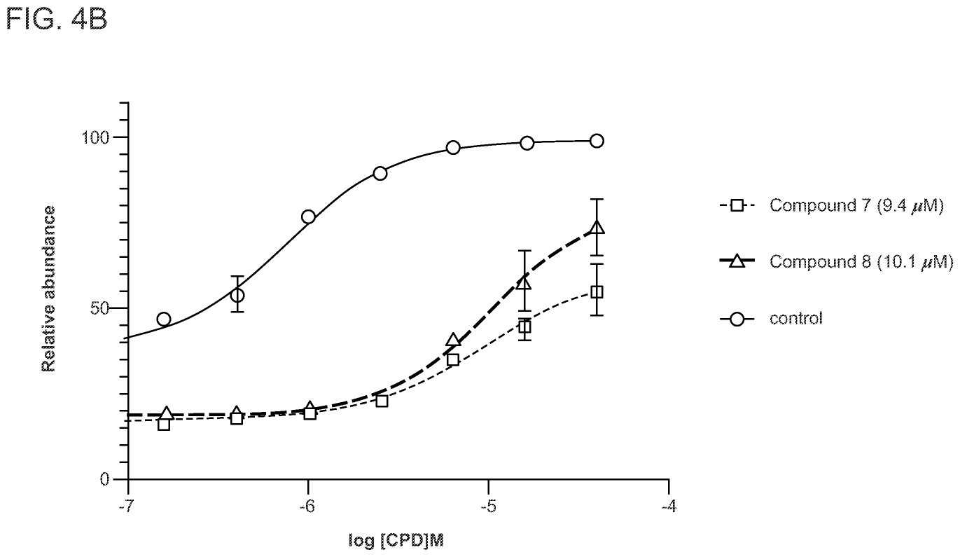

The results of the cellular VHL engagement assay are shown in FIG. 4A-FIG. 4B.

Inventive bifunctional compounds 1, 2, 3, 4, 5, 7, 8, 9, and 10 exhibited IC₅₀ values of 16.9 μM, 11.7 μM, 15.1 μM, 22.9 μM, 19.3 μM, 9.4 μM, 10.1 μM, 298 μM, and 9.0 μM, respectively.

All patent publications and non-patent publications are indicative of the level of skill of those skilled in the art to which this invention pertains. All these publications (including any specific portions thereof that are referenced) are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A compound, comprising an (E)-3-(4-(((2-(1H-indol-3-yl)ethyl)amino)methyl)phenyl)acrylamidyl moiety and a degron covalently attached to each other by a linker, wherein the compound has a structure represented by formula (I) or (II): 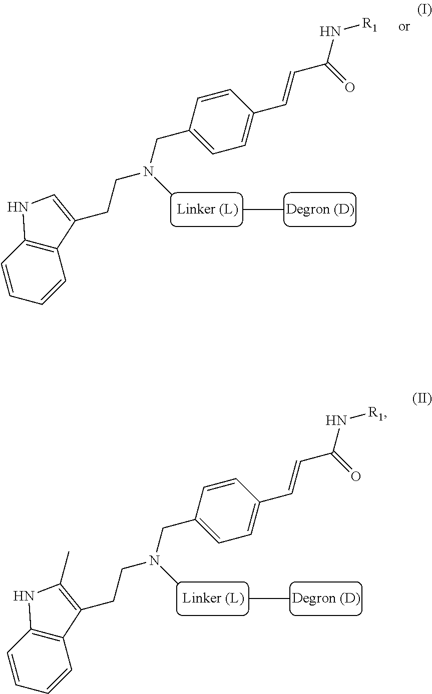
wherein R₁ is OH or 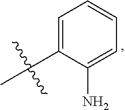 [SMILES: *c1ccccc1N]
and the degron represents a ligand that binds von Hippel Landau tumor suppressor (VHL), or a pharmaceutically acceptable salt or stereoisomer thereof, wherein the bifunctional compound has DC₅₀ (half maximal degradation concentration) for HDAC3 activity that is at least about 1, 2, 3, 4, 5, 6, 7, 8, 9, or 10-fold lower than the DC₅₀ for one or more of HDAC1, HDAC2, HDAC4, HDAC5, HDAC7, HDAC9, and/or HDAC10.

2. The compound of claim 1, wherein the linker comprises an alkylene chain or a bivalent alkylene chain, either of which may be interrupted by, and/or terminate (at either or both termini) at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

3. The compound of claim 2, wherein the alkylene chain comprises 2-12 alkylene units.

4. The compound of claim 1, wherein the linker comprises a polyethylene glycol chain that may be interrupted by, and/or terminate (at either or both termini) at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

5. The compound of claim 4, wherein the polyethylene glycol chain comprises 1-6 PEG units.

6. The compound of claim 1, which is represented by any one of structures (I-1) to (I-7) and (II-1) to (II-7): 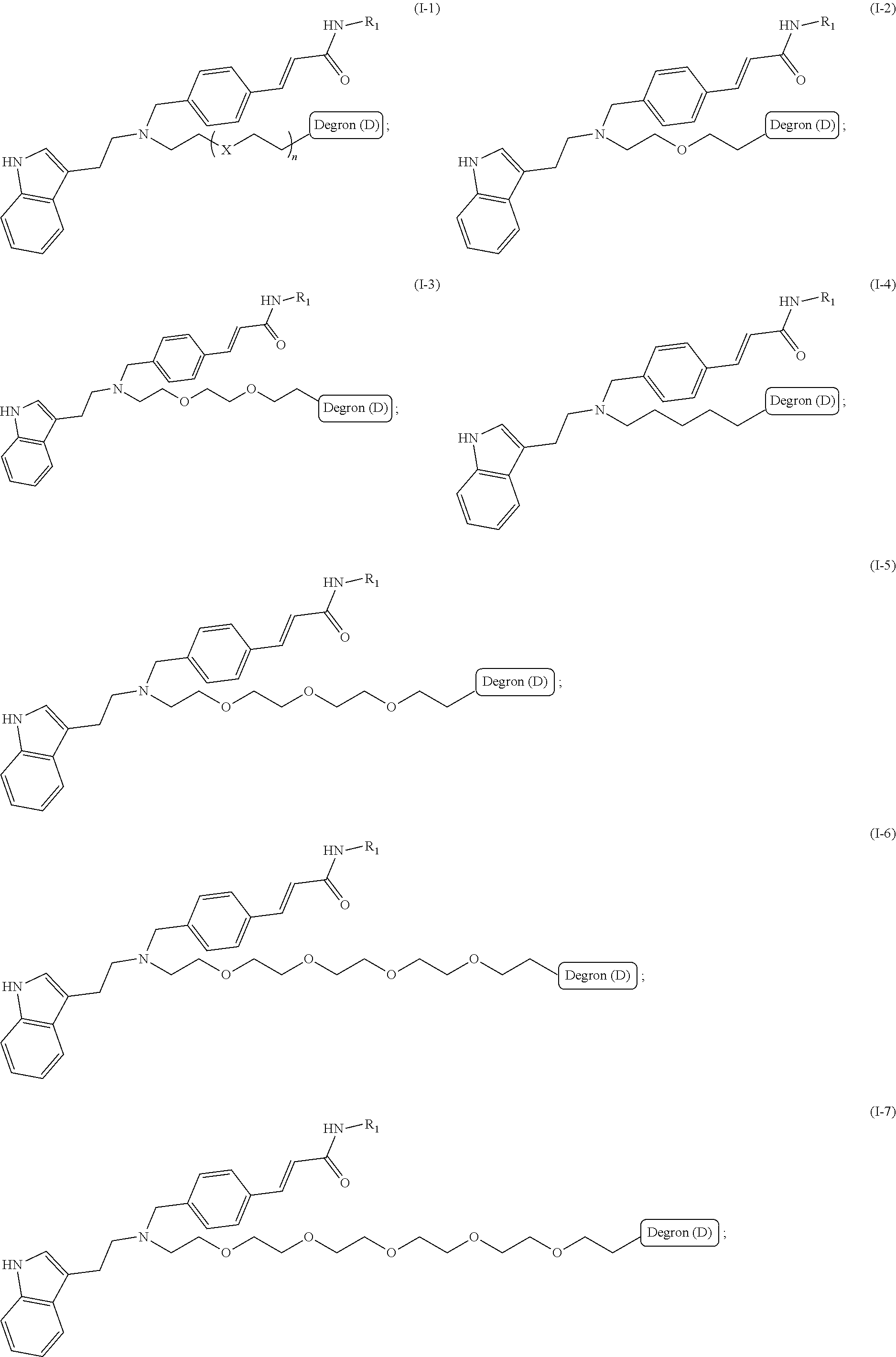 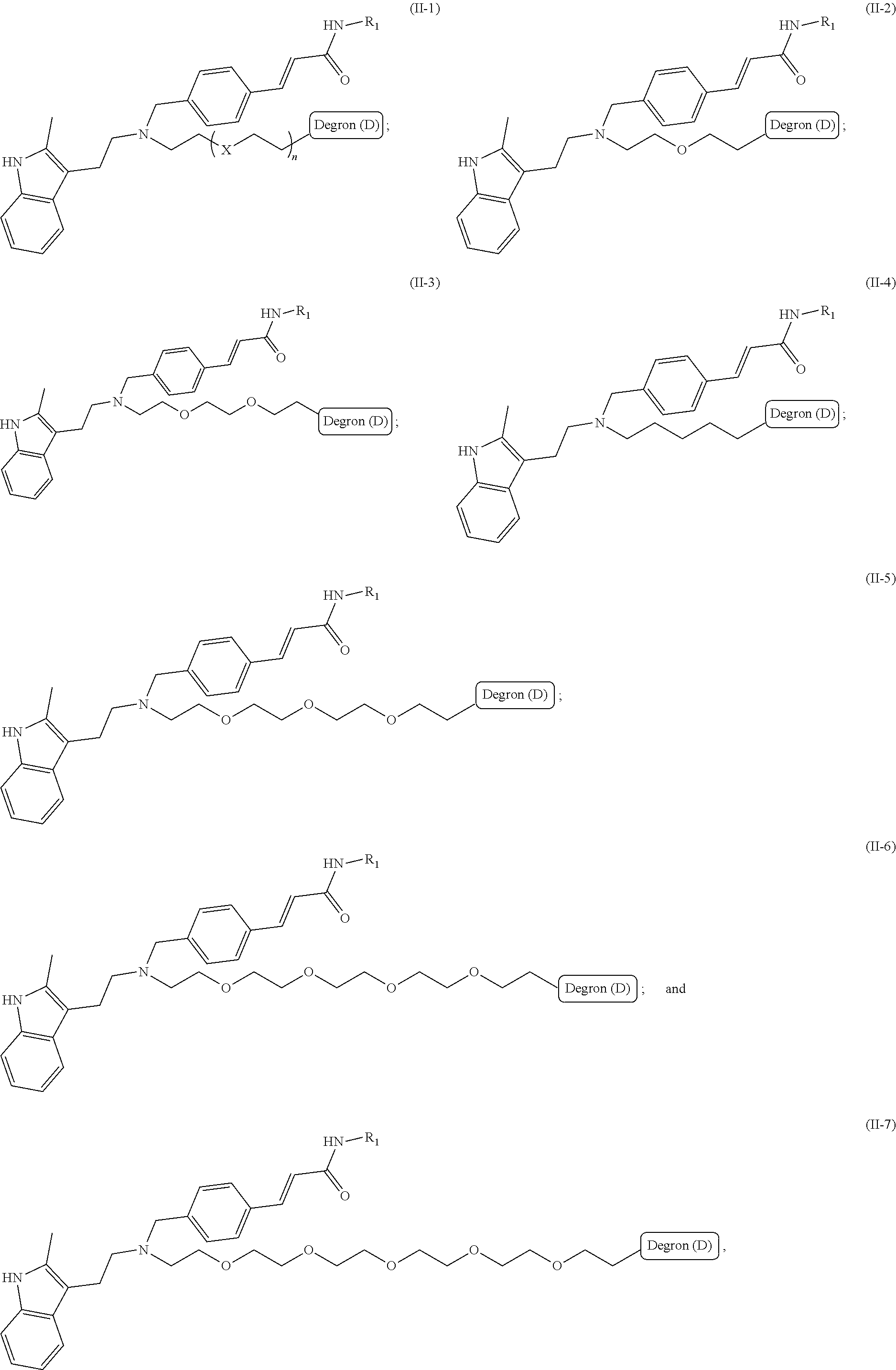
wherein n is an integer from 1-6; R₁ is OH or 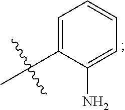 [SMILES: *c1ccccc1N]
and X is O or CH₂, or a pharmaceutically acceptable salt or stereoisomer thereof.

7. The compound of claim 1, wherein the degron is represented by any one of structures (D1-a) to (D1-f): 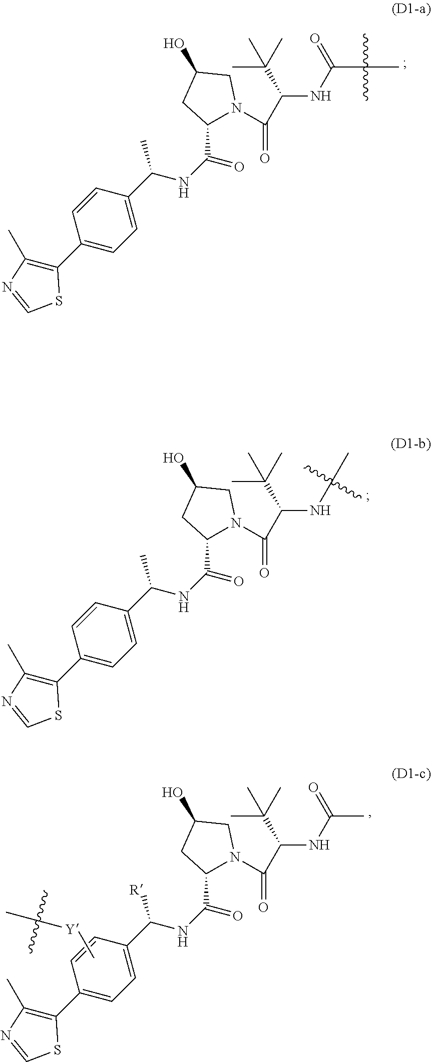
wherein Y′ is a bond, N, O or C and R′ is H or methyl; 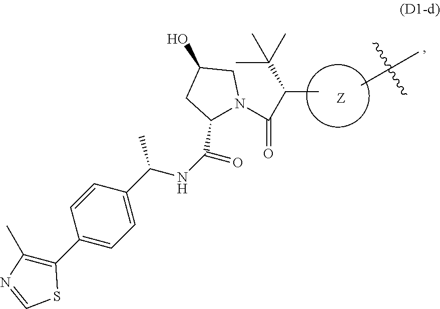
wherein Z is a C₅-C₆ carbocyclic or a C₅-C₆ heterocyclic group; 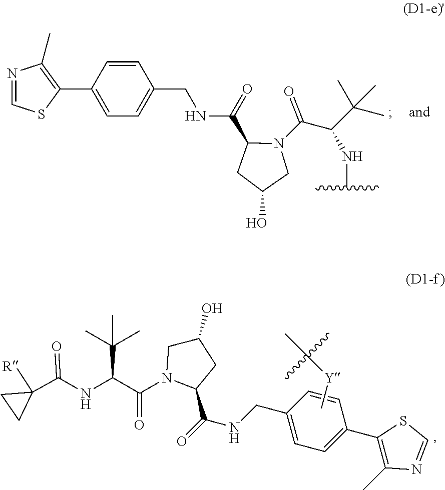
wherein Y″ is a bond, N, O or C and R″ is F or CN,
or a stereoisomer thereof.

8. The compound of claim 7, wherein Z is

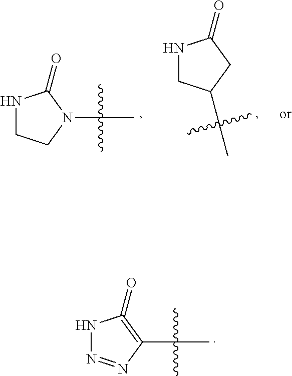

9. The compound of claim 7, which is represented by any one of structures (I-8) to (I-12) and (II-8) to (II-12): 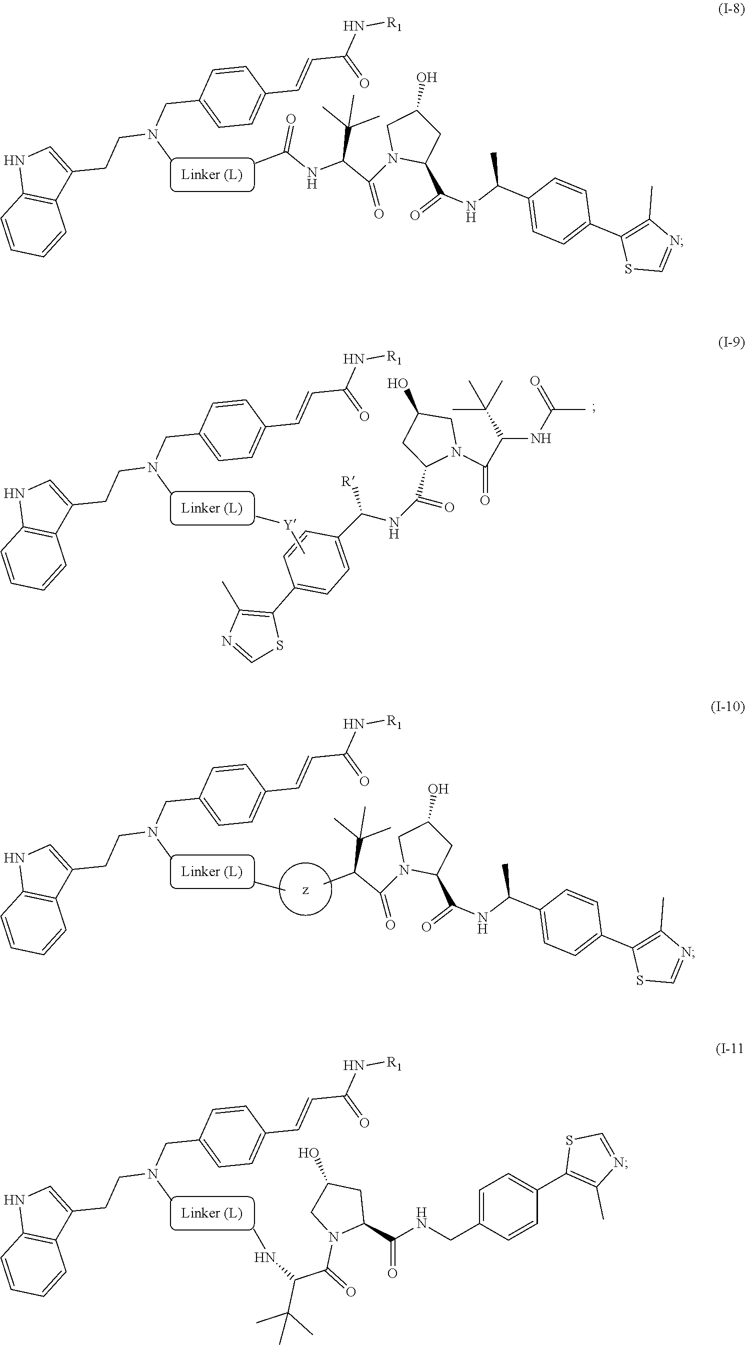 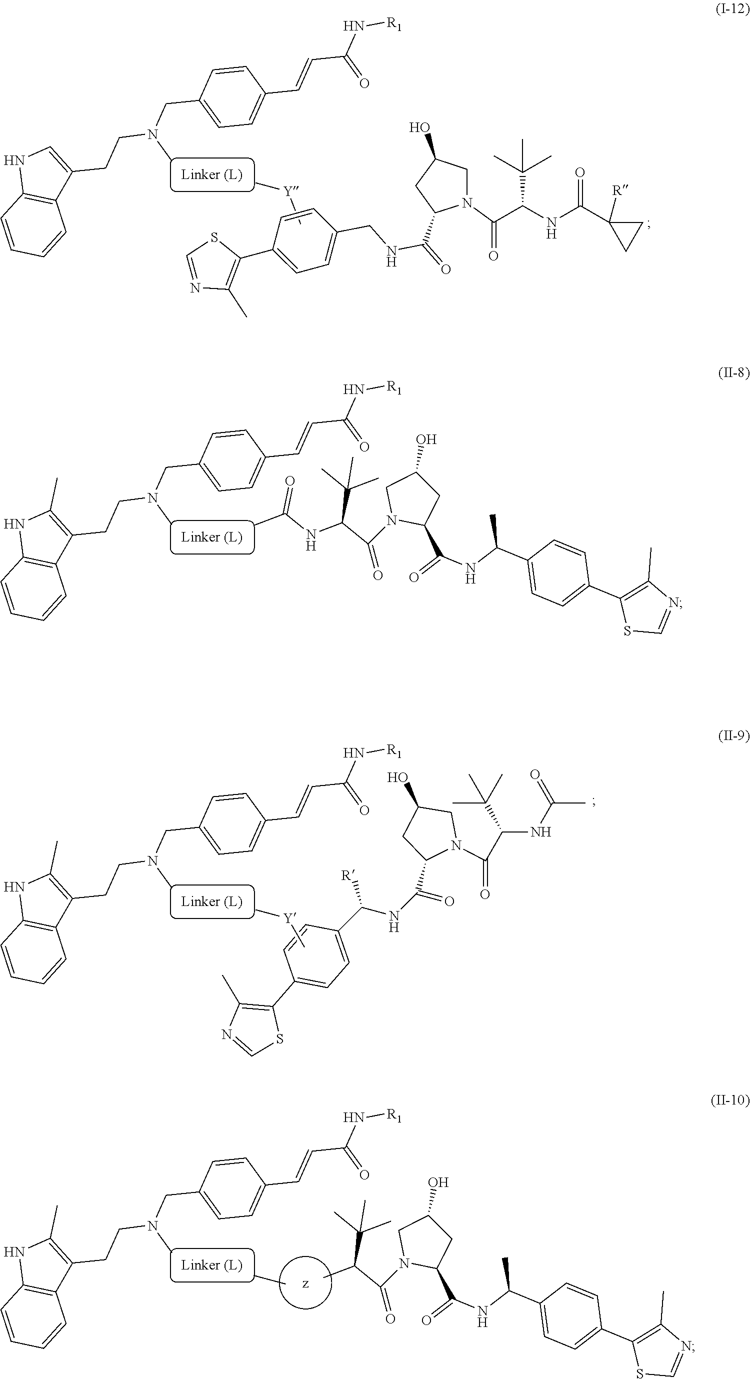 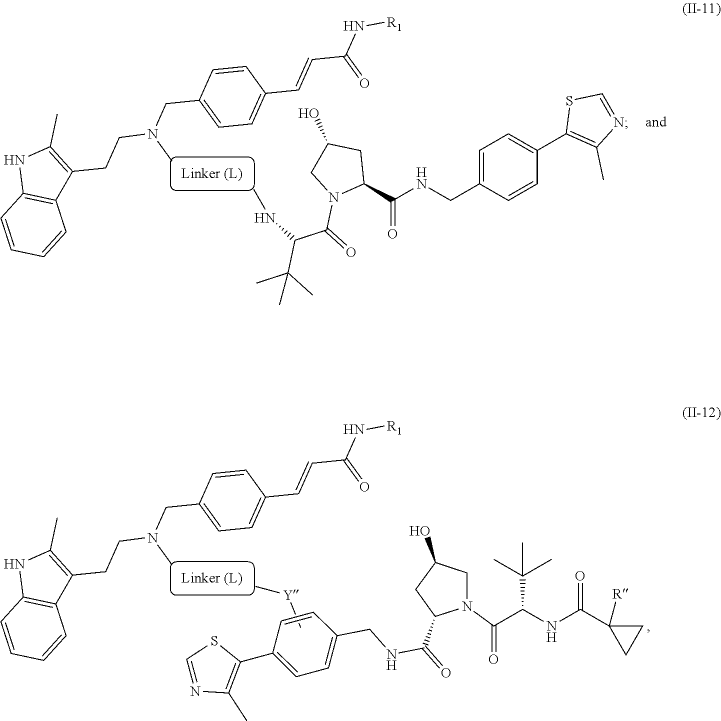
or a pharmaceutically acceptable salt or stereoisomer thereof.

10. The compound of claim 9, which is represented by any one of structures (I-13) to (I-18) and (II-13) to (II-18): 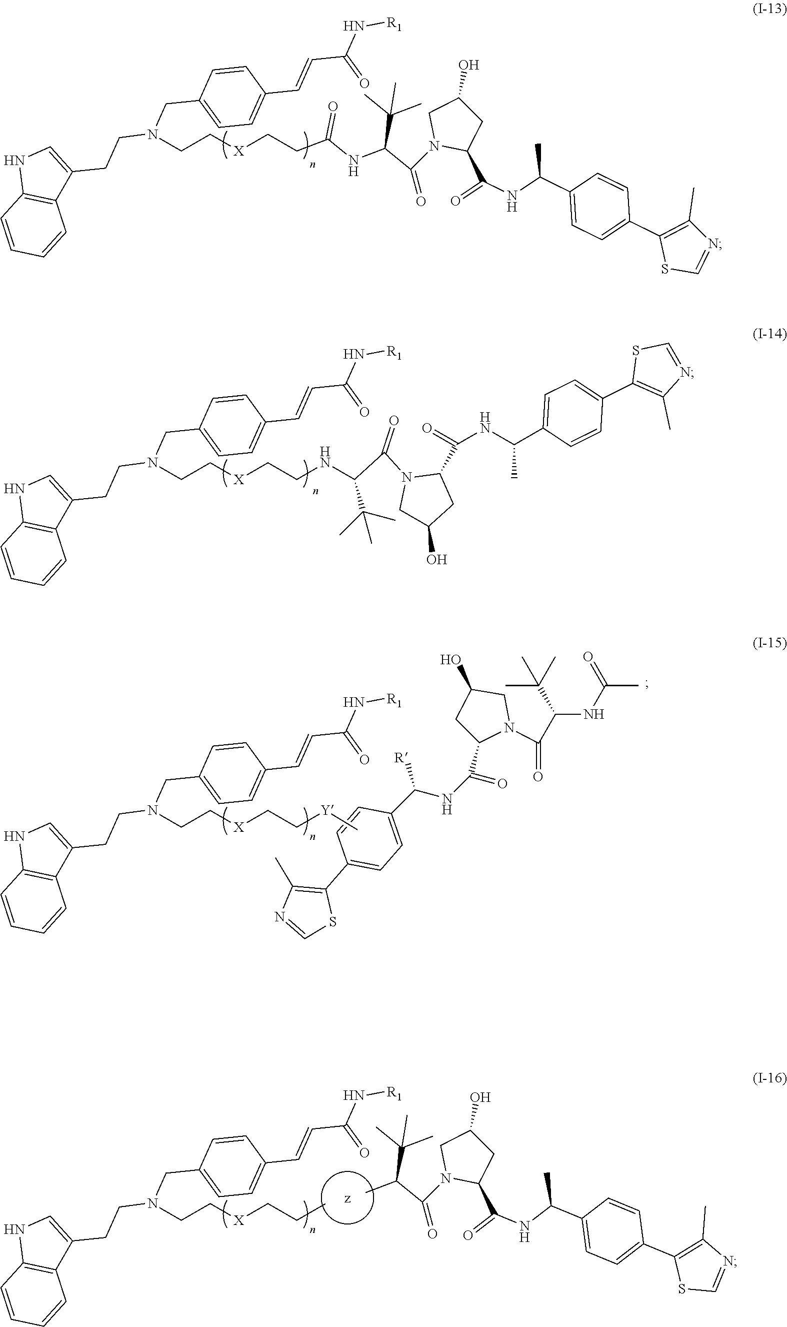 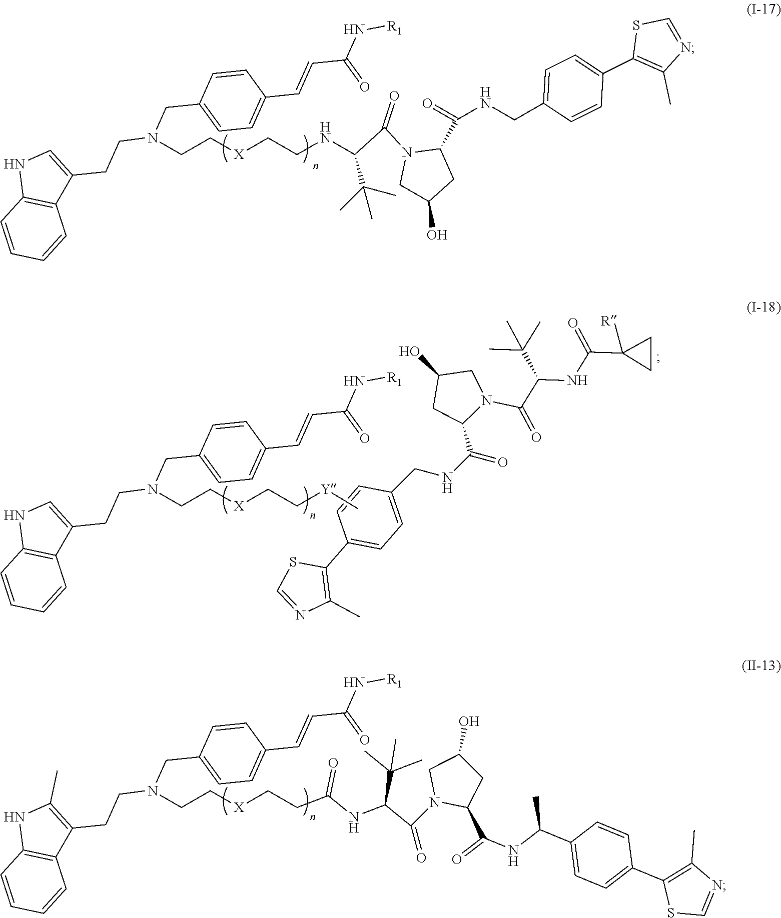 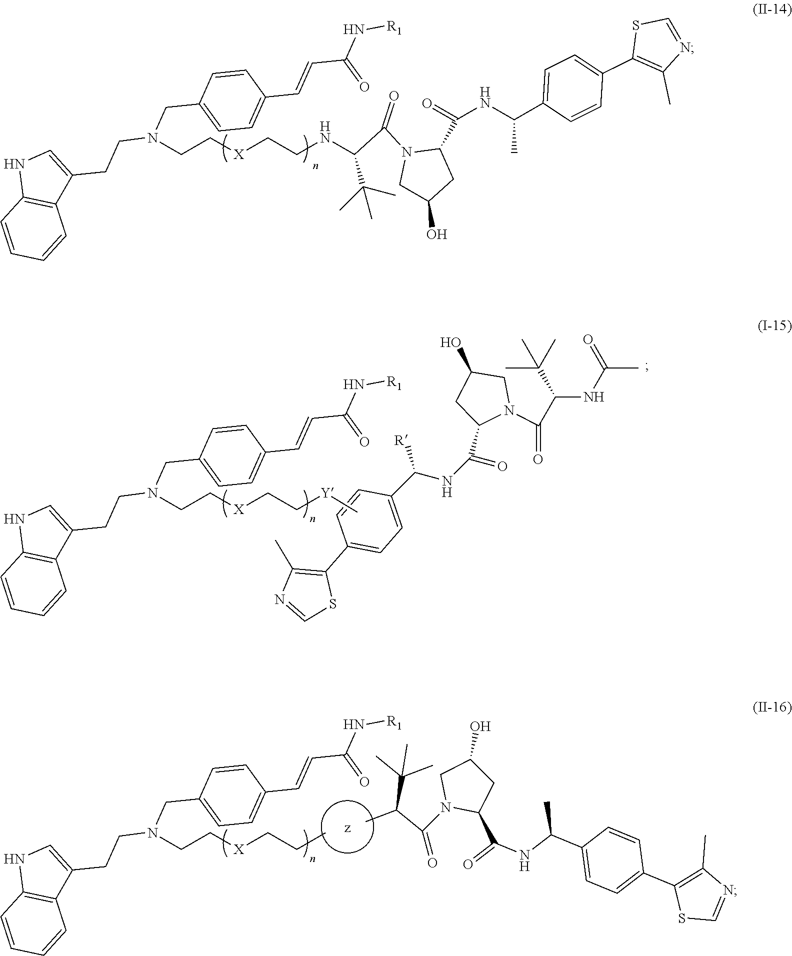 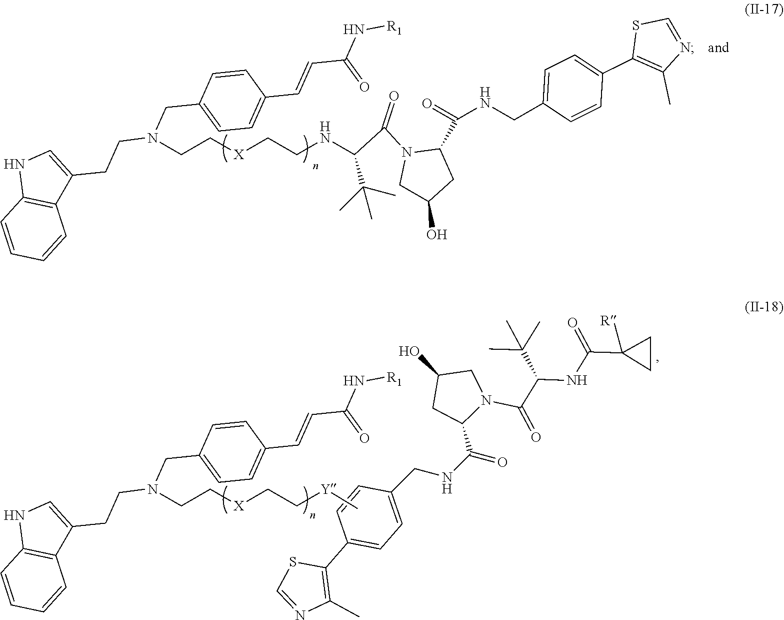
wherein n is an integer from 1-6 and X is O or CH₂, or a pharmaceutically acceptable salt or stereoisomer thereof.

11. The compound of claim 1, wherein R₁ is OH.

12. The compound of claim 1, wherein R₁ is

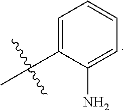 [SMILES: *c1ccccc1N]

13. The compound of claim 1, which is represented by any one of structures (1) to (6) and (8) to (15): 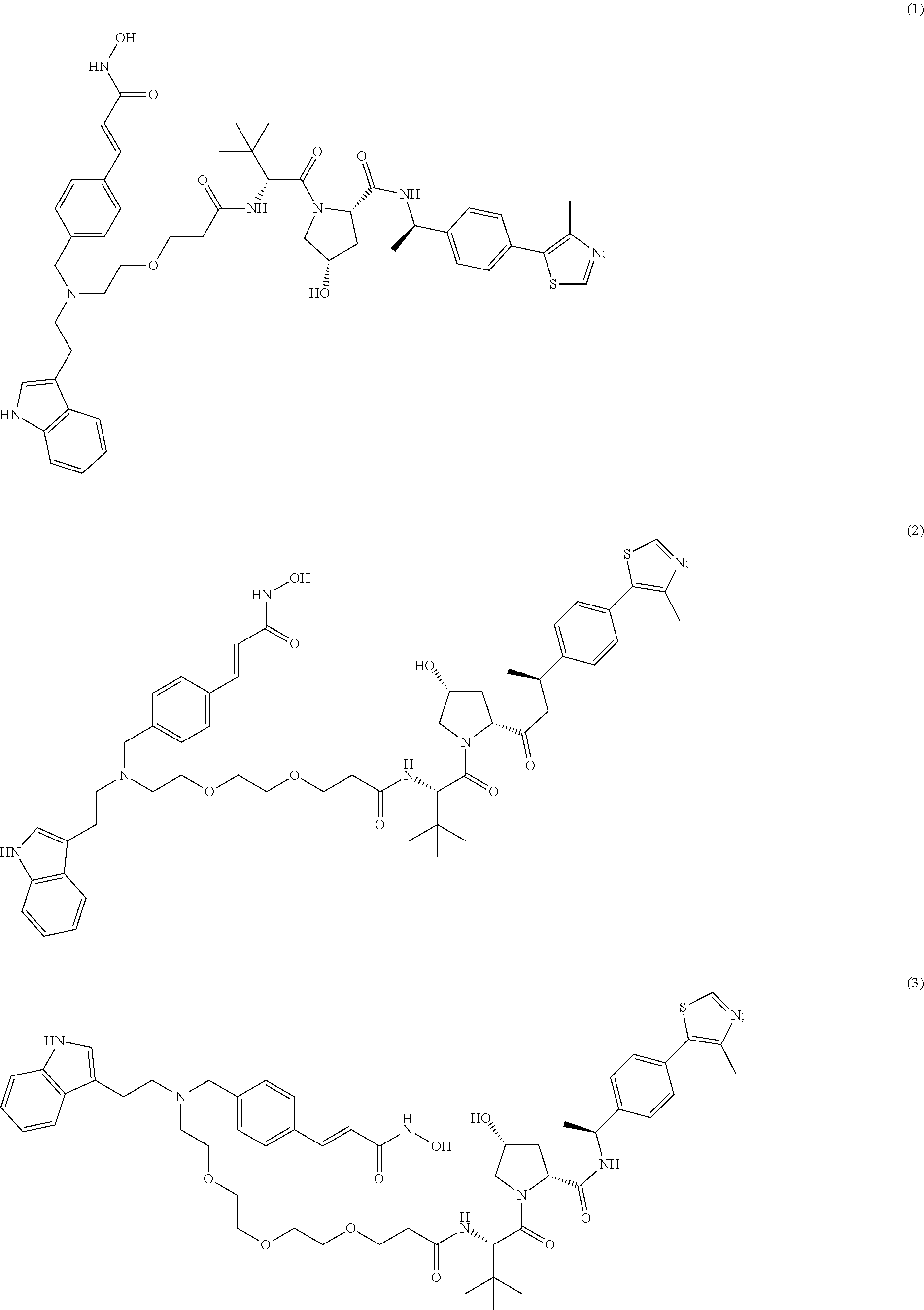 [SMILES: Cc1ncsc1-c1ccc([C@@H](C)NC(=O)[C@@H]2C[C@H](O)CN2C(=O)[C@H](NC(=O)CCOCCN(CCc2c[nH]c3ccccc23)Cc2ccc(/C=C/C(=O)NO)cc2)C(C)(C)C)cc1 ; Cc1ncsc1-c1ccc([C@H](C)CC(=O)[C@H]2C[C@@H](O)CN2C(=O)[C@@H](NC(=O)CCOCCOCCN(CCc2c[nH]c3ccccc23)Cc2ccc(/C=C/C(=O)NO)cc2)C(C)(C)C)cc1 ; Cc1ncsc1-c1ccc([C@H](C)NC(=O)[C@H]2C[C@@H](O)CN2C(=O)[C@@H](NC(=O)CCOCCOCCOCCN(CCc2c[nH]c3ccccc23)Cc2ccc(/C=C/C(=O)NO)cc2)C(C)(C)C)cc1] 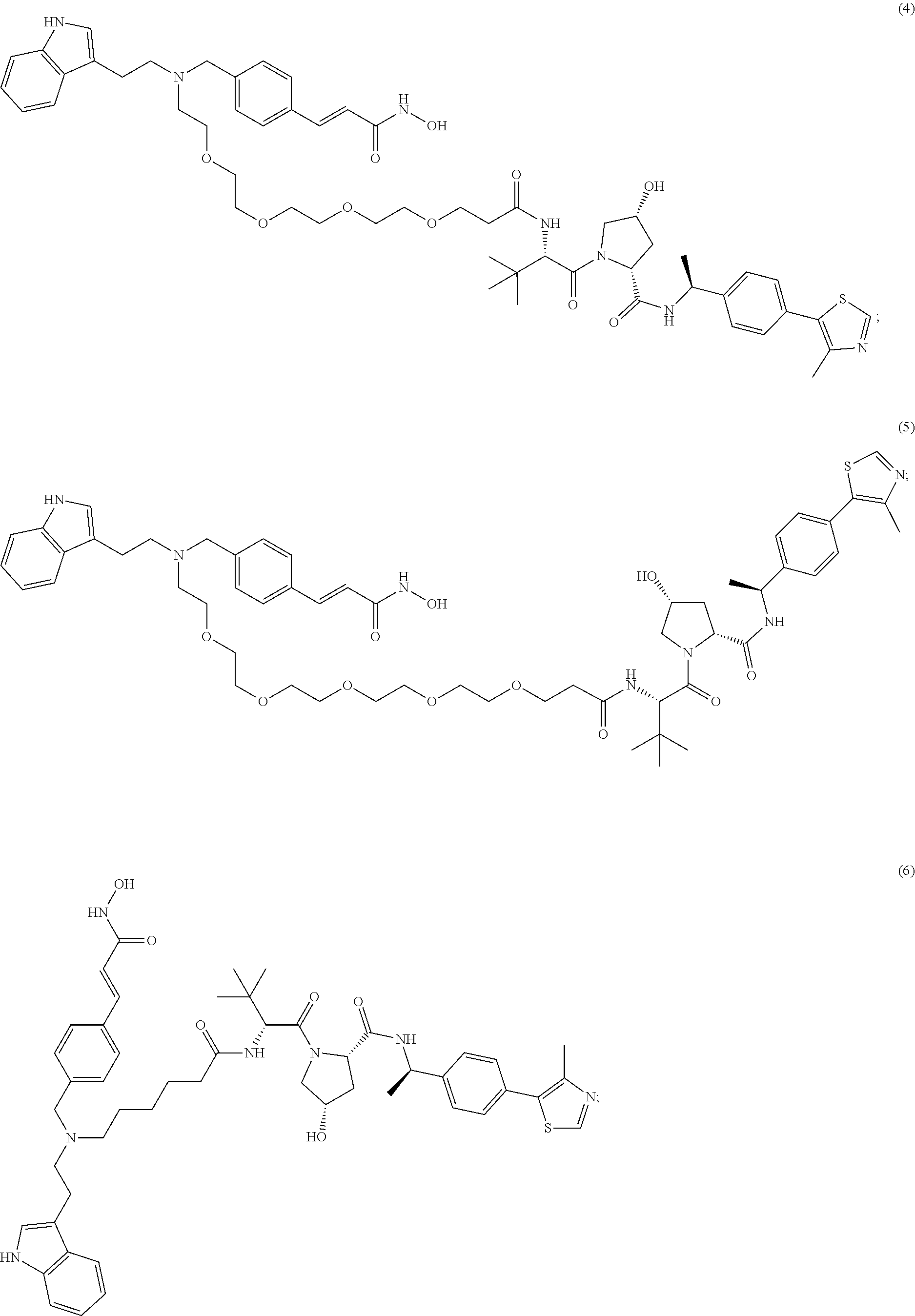 [SMILES: Cc1ncsc1-c1ccc([C@H](C)NC(=O)[C@H]2C[C@@H](O)CN2C(=O)[C@@H](NC(=O)CCOCCOCCOCCOCCN(CCc2c[nH]c3ccccc23)Cc2ccc(/C=C/C(=O)NO)cc2)C(C)(C)C)cc1 ; Cc1ncsc1-c1ccc([C@H](C)NC(=O)[C@H]2C[C@@H](O)CN2C(=O)[C@@H](NC(=O)CCOCCOCCOCCOCCOCCN(CCc2c[nH]c3ccccc23)Cc2ccc(/C=C/C(=O)NO)cc2)C(C)(C)C)cc1 ; Cc1ncsc1-c1ccc([C@@H](C)NC(=O)[C@@H]2C[C@H](O)CN2C(=O)[C@H](NC(=O)CCCCCN(CCc2c[nH]c3ccccc23)Cc2ccc(/C=C/C(=O)NO)cc2)C(C)(C)C)cc1] 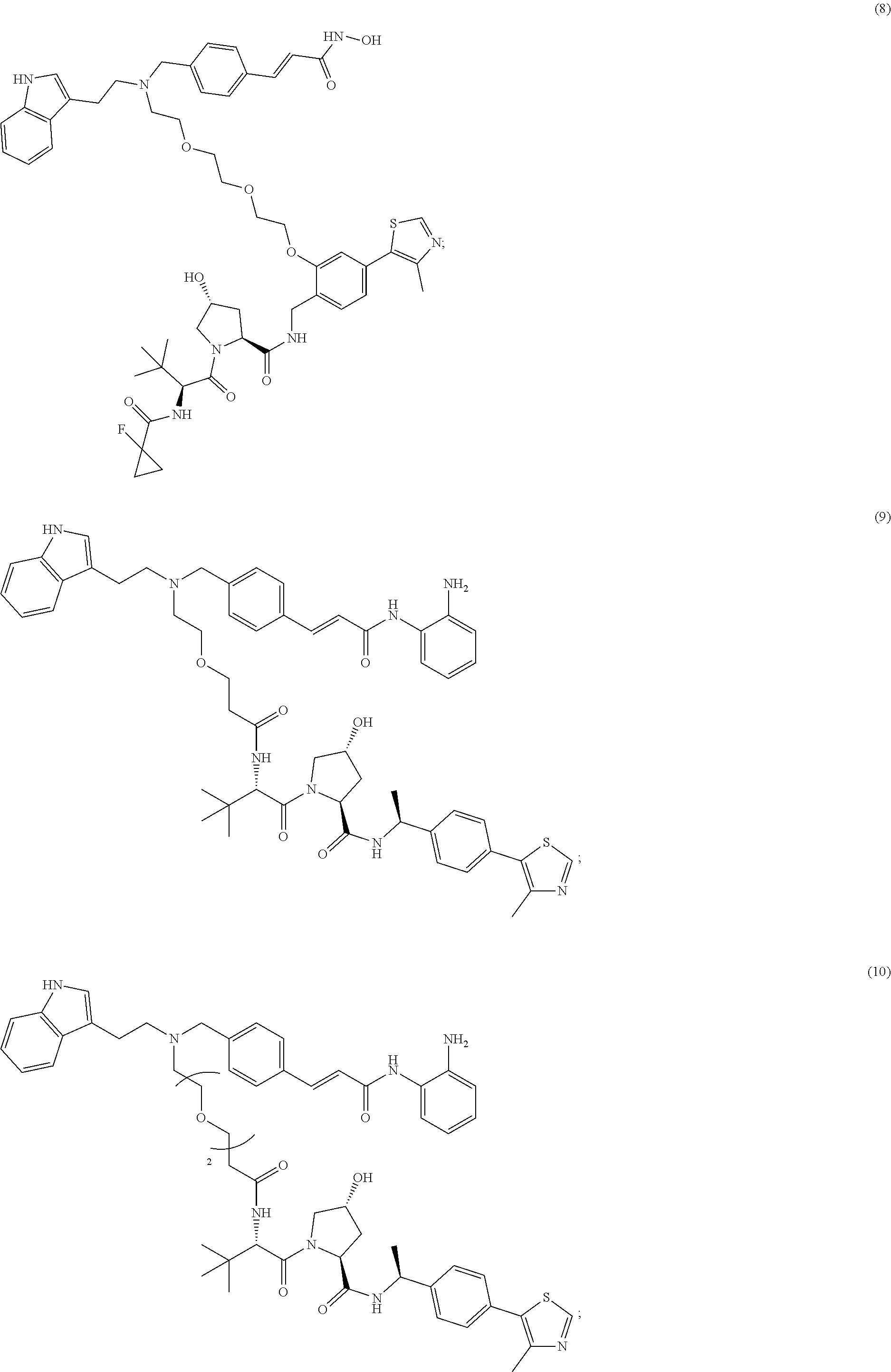 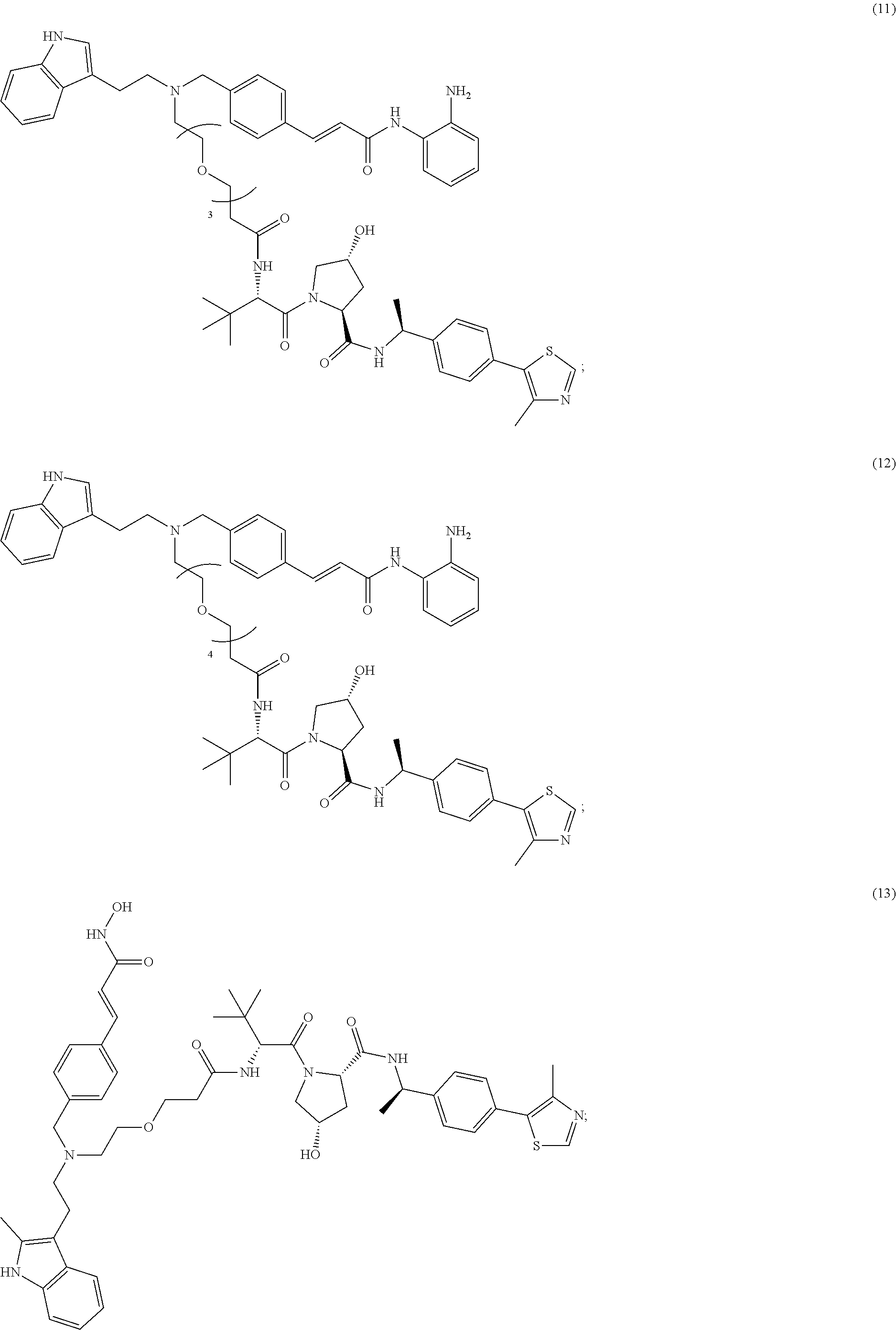 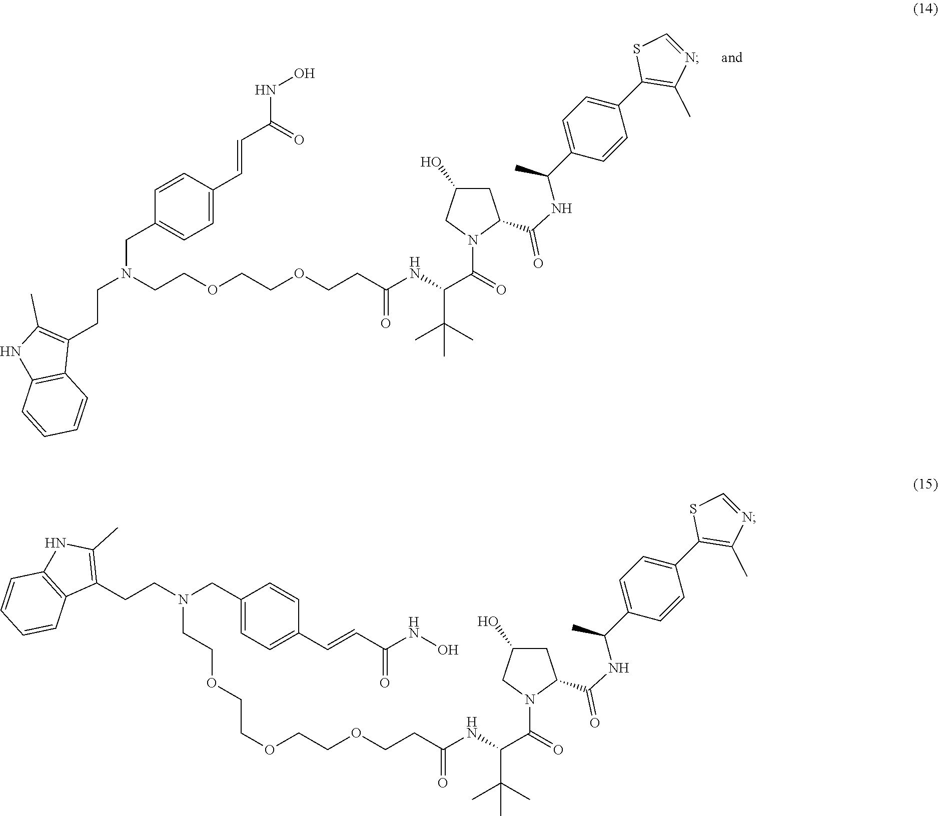 [SMILES: Cc1ncsc1-c1ccc([C@H](C)NC(=O)[C@H]2C[C@@H](O)CN2C(=O)[C@@H](NC(=O)CCOCCOCCN(CCc2c(C)[nH]c3ccccc23)Cc2ccc(/C=C/C(=O)NO)cc2)C(C)(C)C)cc1 ; "and" ; Cc1ncsc1-c1ccc([C@H](C)NC(=O)[C@H]2C[C@@H](O)CN2C(=O)[C@@H](NC(=O)CCOCCOCCOCCN(CCc2c(C)[nH]c3ccccc23)Cc2ccc(/C=C/C(=O)NO)cc2)C(C)(C)C)cc1]
or a pharmaceutically acceptable salt or stereoisomer thereof.

14. A pharmaceutical composition, comprising a therapeutically effective amount of the compound or pharmaceutically acceptable salt or stereoisomer thereof of claim 1, and a pharmaceutically acceptable carrier.

15. A method of treating a disease or disorder that is characterized or mediated by aberrant activity of HDAC3, comprising administering to a subject in need thereof a therapeutically effective amount of the compound or pharmaceutically acceptable salt or stereoisomer thereof of claim 1, wherein the disease or disorder is cancer, a neurodegenerative disease, or an autoimmune disease.

16. (canceled)

17. The method of claim 15, wherein the cancer is breast cancer, prostate cancer, pancreatic cancer, laryngeal cancer, Hodgkin's lymphoma, neuroblastoma, polycythemia vera, T-cell lymphoma, multiple myeloma, leukemia, hepatocellular carcinoma, non-small cell lung cancer, or essential thrombocythemia.

18. (canceled)

19. The method of claim 15, wherein the neurodegenerative disease is Parkinson's disease, Alzheimer's disease, or Huntington's disease.

20. (canceled)

21. The method of claim 15, wherein the autoimmune disease is Sjogren's syndrome, Hashimoto thyroiditis, rheumatoid arthritis, juvenile (type 1) diabetes, polymyositis, scleroderma, Addison disease, lupus including systemic lupus erythematosus, vitiligo, pernicious anemia, glomerulonephritis, pulmonary fibrosis, celiac disease, polymyalgia rheumatica, multiple sclerosis, ankylosing spondylitis, alopecia areata, vasculitis, or temporal arteritis.
